(19) United States
(12) Patent Application Publication
(10) Pub. No.: US 2025/0115880 A1
(43) Pub. Date: Apr. 10, 2025

(54) VIRAL VECTOR PRODUCTION SYSTEM

(71) Applicant: Oxford BioMedica (UK) Limited, Oxford (GB)

(72) Inventors: Daniel Farley, Oxford (GB); Kyriacos Mitrophanous, Oxford (GB)

(21) Appl. No.: 18/817,046

(22) Filed: Aug. 27, 2024

Related U.S. Application Data
(63) Continuation of application No. 16/980,825, filed on Sep. 14, 2020, now abandoned.
(60) Provisional application No. 62/795,222, filed on Jan. 22, 2019.
(60) Provisional application No. 62/644,373, filed on Mar. 16, 2018.

(30) Foreign Application Priority Data
Sep. 7, 2018 (GB) 1814590.4

(51) Int. Cl. C12N 15/86; C12N 9/22

(52) U.S. Cl. CPC C12N 7/00; C12N 5/0686; C12N 9/22; C12N 2502/99; C12N 2510/02; C12N 2740/15051; C12N 2750/14151

(57) ABSTRACT

Disclosed herein are viral vector production systems secreting nuclease for degradation of residual nucleic acid during viral vector production and methods of the same. Such a viral vector production system comprises a viral vector production cell comprising nucleic acid sequences encoding: 1) viral vector components; and 2) a nuclease, wherein the nuclease is expressed in the production cell and secreted in cell culture thereby degrading residual nucleic acid during viral vector production. Another such viral vector production system comprises 1) a viral vector production cell comprising nucleic acid sequences encoding viral vector components; and 2) a nuclease helper cell comprising a nucleic acid sequence encoding a nuclease, wherein the nuclease is expressed and secreted in co-culture of the production cell of 1) and the helper cell of 2), thereby degrading residual nucleic acid during viral vector production.

### FIELD OF THE INVENTION

The invention relates to the production of viral vectors. In particular, the invention relates to viral vector cell production systems engineered to express and secrete a nuclease into cell culture media during the vector manufacturing process.

### SEQUENCE LISTING

The instant application contains a Sequence Listing, which is hereby incorporated by reference in its entirety. Said .xml copy, created on Dec. 11, 2024, is named OBM008US2, and is 32,029 bytes in size.

### BACKGROUND TO THE INVENTION

As indicated above, the present invention relates to production cells, the preparation thereof and uses thereof. A production cell is sometimes also referred to as a host cell or host production cell. The production cells are useful in inter alia gene therapy.

Gene therapy broadly involves the use of genetic material to treat disease. It includes the supplementation of cells with defective genes (e.g. those harbouring mutations) with functional copies of those genes, the inactivation of improperly functioning genes and the introduction of new therapeutic genes.

Therapeutic genetic material may be incorporated into the target cells of a host using vectors to enable the transfer of nucleic acids. Such vectors can be generally divided into viral and non-viral categories.

Viruses naturally introduce their genetic material into target cells of a host as part of their replication cycle. Engineered viral vectors harness this ability to enable the delivery of a nucleotide of interest (NOI) to a target cell. To date, a number of viruses have been engineered as vectors for gene therapy. These include retroviruses, adenoviruses (AdV), adeno-associated viruses (AAV), herpes simplex viruses (HSV) and vaccinia viruses.

In addition to modification to carry a nucleotide of interest, viral vectors are typically further engineered to be replication defective. As such, the recombinant vectors can directly infect a target cell, but are incapable of producing further generations of infective virions. Other types of viral vectors may be conditionally replication competent within cancer cells only, and may additionally encode a toxic transgene or pro-enzyme.

The use of viral vectors for delivery of therapeutic genes is well known and wide-ranging across indications. In particular, gene therapy advances and products are now an important part of our global healthcare markets. Contemporary gene therapy vectors based on RNA viruses such as γ-Retroviruses and Lentiviruses, and DNA viruses such as Adenovirus and Adeno-associated virus (AAV) have shown promise in a growing number of human disease indications. These include ex vivo modification of patient cells for haematological conditions, and in vivo treatment of ophthalmic, cardiovascular, neurodegenerative diseases and tumor therapy or immunotherapy. Other viral vectors such as viruses based on Poxviruses and Avian viruses are widely used in human and animal vaccinations.

Retroviral vectors, developed as therapies for various genetic disorders, continue to show increasing promise in clinical trials and now a few form the basis of approved therapeutic products. Currently there are over 459 human clinical trials involving retroviral gene therapy registered in the Journal of Gene Medicine database; 158 gene therapy clinical trials are using lentiviral vectors (http://www.abedia.com/wiley/vectors.php, updated in April, 2017). Strimvelis received marketing authorisation from the European Commission on 26 May 2016; Strimvelis is a product for treatment of ADA-SCID based on patient CD34⁺ cells transduced ex vivo with retroviral vectors expressing the ADA gene. Kymriah (USAN: tisagenlecleucel) received approval from the FDA on 30 Aug. 2017; Kymriah is a product for the treatment of patients up to 25 years old with refractory ALL. Papers on retroviral gene therapy include Wang X, Naranjo A, Brown C E, Bautista C, Wong C W, Chang W C, Aguilar B, Ostberg J R, Riddell S R, Forman S J, Jensen M C (2012) J Immunother. 35(9):689-701, Hu Y, Wu Z, Luo Y, Shi J, Yu J, Pu C, Liang Z, Wei G, Cui Q, Sun J, Jiang J, Xie J, Tan Y, Ni W, Tu J, Wang J, Jin A, Zhang H, Cai Z, Xiao L, Huang H. (2017) Clin Cancer Res. 23(13):3297-3306, Galy, A. and A. J. Thrasher (2010) Curr Opin Allergy Clin Immunol 11(6): 545-550; Porter, D. L., B. L. Levine, M. Kalos, A. Bagg and C. H. June (2011) N Engl J Med 365(8): 725-733; Campochiaro, P. A. (2012) Gene Ther 19(2): 121-126; Cartier, N., S. Hacein-Bey-Abina, C. C. Bartholomae, P. Bougneres, M. Schmidt, C. V. Kalle, A. Fischer, M. Cavazzana-Calvo and P. Aubourg (2012) Methods Enzymol 507: 187-198; Sadelain, M., I. Riviere, X. Wang, F. Boulad, S. Prockop, P. Giardina, A. Maggio, R. Galanello, F. Locatelli and E. Yannaki (2010) Ann N Y Acad Sci 1202: 52-58; DiGiusto, D. L., A. Krishnan, L. Li, H. Li, S. Li, A. Rao, S. Mi, P. Yam, S. Stinson, M. Kalos, J. Alvarnas, S. F. Lacey, J. K. Yee, M. Li, L. Couture, D. Hsu, S. J. Forman, J. J. Rossi and J. A. Zaia (2010) Sci Transl Med 2(36): 36ra43 and Segura M M, M. M., Gaillet B, Garnier A. (2013) Expert opinion in biological therapy).

Important examples of such vectors include the gamma-retrovirus vector system (based on MMLV), the primate lentivirus vector system (based on HIV-1) and the non-primate lentivirus vector system (based on EIAV).

Reverse genetics has allowed these virus-based vectors to be heavily engineered such that vectors encoding large heterologous sequences (circa 10 kb) can be produced by transfection of mammalian cells with appropriate DNA sequences (reviewed in Bannert, K. (2010) Caister Academic Press: 347-370).

Engineering and use of retroviral vectors at the research stage typically involve the production of reporter-gene vectors encoding, for example, GFP or lacZ. The titres of these clinically irrelevant vectors are usually in the region of 1×10⁶ to 1×10⁷ transducing units per mL (TU/mL) of crude harvest material.

The manufacture of viral vectors for human gene therapy and vaccination is well documented over the last several decades in scientific journals. Well known methods of viral vector manufacture include the transfection, such as transient transfection, of primary cells or mammalian/insect cell lines with vector DNA components, followed by a limited incubation period and then harvest of crude vector from culture media and/or cells. Transient transfection requires that the viral genes necessary for the production of viral vectors are introduced into a production cell (for example, HEK-293) via plasmids by transfection. Often, each component required for vector production is encoded by separate plasmids, partly for safety reasons, as it would then require a number of recombination events to occur for a replication competent virus particle to be formed through the production process.

After transfection, incubation & harvest, viral vector virions are then purified and concentrated from crude material using a generalized process of some or all of the following steps: 1) clarification, 2) digest of unwanted or contaminating nucleic acid (e.g., Benzonase® treatment), 3) column chromatography (e.g., ion exchange), 4) buffer exchange/concentration (e.g., ultrafiltration) and further nucleic acid removal (e.g., second Benzonase® treatment), 5) polishing (e.g., size exclusion) and 6) sterile filtration.

Despite decades of refinement, transient transfection has inherent drawbacks. The cost of transfection agents/plasmids, and/or process agents are high and, coupled with the labour-intensive nature of the transfection technique, this makes transient transfection an expensive and technically complex process for clinical/commercial vector production.

Thus, there is a desire in the art to provide alternative methods of producing viral vectors which help to address the known issues associated with the transient transfection process.

There has been an attempt to generate stable packaging cell lines in recent years, where viral packaging genes are introduced into eukaryotic host cells along with selection markers such that these genes can be stably integrated into the cell. Similarly, stable producer cell lines also exist where the retroviral genome is also stably integrated. Both of these allow circumvention of a significant portion of the transient transfection process.

Reference is also made to co-pending EP 17210359.0 application number entitled RETROVIRAL VECTOR, incorporated by reference in its entirety herein, and which describes viral vector production systems and methods employing modular constructs comprising at least two of the nucleic acid components necessary for viral vector production. Such modular constructs were found to provide levels of vector production comparable to those with a traditional multi-plasmid transient process, but allowing for a significant reduction in, for example, the use of transfection agents.

Thus, there is a desire in the vector manufacturing sector to improve both transient vector production processes as well as the processes for generating stable packaging and producer cell lines.

The removal of nucleic acids, derived from either production cells or viral vector components, from the final drug product is an important aspect of safety and an area of viral vector manufacturing ripe for improvement. When transformed eukaryotic cell lines, such as HEK293T cells (which also contain Adenovirus E1 and SV40 T antigen genes) are used for production, these cells typically harbour genes with (proto)-oncogenic properties. The inevitable cell death and release of production cell DNA during viral vector manufacture leads to the presence of such (partial and contaminating) sequences within crude harvest material. It is therefore desirable to minimize general DNA contamination (e.g., longer contaminating dsDNA is degraded to short forms within the final product) to preclude the potential for unnecessary & potentially harmful functional gene sequences from being integrated into patient cells during vector delivery. In addition, typical viral vector production methods that transiently transfect production cells with large quantities of plasmid DNA (pDNA) encoding the viral vector components will result in the majority of the contaminating DNA being of vector component origin. As such, it is also desirable to remove contaminating, residual pDNA that could otherwise be taken up and expressed by patient cells.

From lab scale to industrial scale, viral vector manufacturers have turned to the use of recombinant nucleases, such as derived from Serratia marcescens (e.g., Benzonase®) or other commercial hydrolytic nucleases, to treat crude vector material and remove contaminating nucleic acid during the upstream and downstream purification processes. For nuclease treatment during upstream processing, a recombinant nuclease in protein form is typically added to harvest material followed by incubation at less than or equal to 37° for a limited period of time. This, however, represents a potentially avoidable additional processing step, and one in which elevated temperature and increased incubation time may lead to loss in vector stability. To avoid this as a stand-alone step after harvest, a recombinant nuclease is often added into the production cell culture at latter stages of vector production. However, if the half-life of the nuclease is relatively short or activity is insufficient to degrade the required amount of residual nucleic acid, a large amount of nuclease may need to be added at once or continually during these latter stages of culturing. Unfortunately, the use of commercially available recombinant nuclease at this stage of the process often becomes practically burdensome and cost-prohibitive, particularly as scale is increased to hundreds or thousands of litres.

Thus, there is a desire to improve viral vector production and manufacturing processes so as to streamline and make more efficient the critical step(s) of degrading residual nucleic acid during viral vector production.

### SUMMARY OF THE INVENTION

The invention disclosed herein describes highly effective and streamlined viral vector production systems and manufacturing processes employing the expression and secretion of a hydrolytic nuclease(s) in viral vector production cells during the production of viral vectors. As such, secretion of a nuclease degrades unwanted or contaminating (residual) nucleic acid during viral vector production. In such vector production systems & processes, the nuclease, encoded by a nucleotide expression cassette, is either transiently co-transfected with the viral vector component expression cassette(s) or stably integrated within a production cell genome or nuclease helper cell genome, or nuclease helper cells may be generated by transient transfection with the nuclease expression cassette.

Due to the complexity of the viral vector production process, expression and secretion of a nuclease from a gene expression cassette in conjunction with a viral vector production system and/or during the viral vector production process is contrary to the state of the art which demonstrates only the expression/co-expression of nuclease in bacterial cells and which, in a viral vector context employs the use of adding commercially available nuclease as a recombinant protein-based enzymatic treatment of viral vector at burdensome upstream and/or downstream time points. It was therefore considered, prior to the invention disclosed herein, that expression and secretion of a nuclease encoded by a nucleic acid expression cassette in conjunction with viral vector component(s) expression cassette(s) in a viral vector production system would result in degradation of necessary vector component DNA thus leading to reduced expression of the vector components, low titres of produced viral vector, and/or toxicity to the viral vector production cells.

However, in an effort to improve viral vector production manufacturing, the inventors provide the invention disclosed herein: viral vector production systems and methods of producing viral vector employing the expression and secretion of a nuclease in viral vector production cells (producer and/or nuclease helper cells) during the production of therapeutic viral vectors. The invention offers a more efficient (and cost-effective) viral vector production system/method compared to current commercial nuclease techniques. Moreover, the invention provides the manufacturing of viral vectors in a streamlined manner, compared to conventional vector manufacturing techniques, by engineering the secretion of a nuclease for degradation of unwanted or contaminating residual nucleic acid during viral vector production without impacting viral titers and/or imparting toxicity to viral vector production cells. The inventors show that for lentiviral vector production the application of secreted nuclease in the upstream phase of production can lead to such efficient degradation of residual DNA within crude harvest material, that further nuclease treatment during the downstream process is made unnecessary. Importantly, the levels of residual DNA within purified/concentrated lentiviral vector material can be greatly reduced when employing secreted nucleases compared to standard commercial nucleases, which is of great significance in the gene therapy field as regulators require ever-increasing improvements in product quality/safety.

Accordingly, disclosed herein is a viral vector production system engineered to secrete a nuclease.

In one embodiment, the viral vector production system comprises a viral production cell comprising nucleic acid sequences encoding: 1) viral vector components; and 2) a nuclease, wherein the nuclease is expressed in the production cell and secreted in cell culture thereby degrading residual nucleic acid during viral vector production.

In another embodiment, the viral vector production system comprises 1) a viral production cell comprising nucleic acid sequences encoding viral vector components; and 2) a nuclease helper cell comprising a nucleic acid sequence encoding a nuclease, wherein the nuclease is expressed and secreted in co-culture of the production cell of 1) and the helper cell of 2), thereby degrading residual nucleic acid during viral vector production.

Also, disclosed herein are methods of producing a viral vector, the methods comprising expressing and secreting a nuclease during viral vector production. In particular, such a method of producing a viral vector comprises the steps of: transfecting (sometimes referred to as contacting) a viral production cell with nucleic acid sequences encoding: 1) viral vector components; and 2) a nuclease, wherein the nuclease is expressed in the production cell and secreted in cell culture thereby degrading residual nucleic acid during viral vector production.

Additionally, disclosed herein is a method of co-culture comprising contacting a viral vector production cell expressing vector components with a nuclease helper cell expressing a nuclease. In a further embodiment, a liquid feed from the helper cell is contacted with a viral vector production cell expressing vector components.

In another embodiment of the invention disclosed herein, is an improved method of producing a viral vector, the improvement comprising introducing nucleic acid sequences into a viral vector production cell, wherein the nucleic acid sequences encode: 1) viral vector components; and 2) a nuclease, wherein the nuclease is expressed in the production cell and secreted in cell culture thereby degrading residual nucleic acid during viral vector production.

Also disclosed herein is an improved method of producing a viral vector, the improvement comprising contacting in co-culture a viral vector production cell comprising viral vector components with a nuclease helper cell expressing a nuclease, wherein the nuclease is expressed in the helper cell and secreted in the cell co-culture thereby degrading residual nucleic acid during viral vector production.

In another embodiment of the viral vector production system or methods disclosed herein, cell culture is maintained in a pH range of 6.5 pH to 7.2 pH.

In another embodiment of the viral vector production system or methods disclosed herein, the nuclease is a sugar-non-specific nuclease.

In another embodiment of the viral vector production system or methods or other aspects of the present invention disclosed herein, the nuclease activity achieved within the main viral vector production vessel is at least about 1 Benzonase® unit equivalents per mL, preferably wherein said nuclease activity is determinable by the DNAse Alert™ assay provided herein as Assay 1.

In another embodiment of the viral vector production system or methods or other aspects of the present invention disclosed herein, the nuclease activity achieved within the main viral vector production vessel is at least about 10 Benzonase® unit equivalents per mL, preferably wherein said nuclease activity is determinable by the DNAse Alert™ assay provided herein as Assay 1.

In another embodiment of the viral vector production system or methods or other aspects of the present invention disclosed herein, the nuclease activity achieved within the main viral vector production vessel is at least about 50 Benzonase® unit equivalents per mL, preferably wherein said nuclease activity is determinable by the DNAse Alert™ assay provided herein as Assay 1.

In another embodiment of the viral vector production system or methods or other aspects of the present invention disclosed herein, the nuclease activity achieved within the main viral vector production vessel is at least about 100 Benzonase® unit equivalents per mL, preferably wherein said nuclease activity is determinable by the DNAse Alert™ assay provided herein as Assay 1.

In another embodiment of the viral vector production system or methods or other aspects of the present invention disclosed herein, the nuclease activity achieved within nuclease helper cell cultures prior to inoculating the main viral vector production vessel is at least about 10 Benzonase® unit equivalents per mL, preferably wherein said nuclease activity is determinable by the DNAse Alert™ assay provided herein as Assay 1.

In another embodiment of the viral vector production system or methods or other aspects of the present invention disclosed herein, the nuclease activity achieved within nuclease helper cell cultures prior to inoculating the main viral vector production vessel is at least about 50 Benzonase® unit equivalents per mL, preferably wherein said nuclease activity is determinable by the DNAse Alert™ assay provided herein as Assay 1.

In another embodiment of the viral vector production system or methods or other aspects of the present invention disclosed herein, the nuclease activity achieved within nuclease helper cell cultures prior to inoculating the main viral vector production vessel is at least about 100 Benzonase® unit equivalents per mL, preferably wherein said nuclease activity is determinable by the DNAse Alert™ assay provided herein as Assay 1.

In another embodiment of the viral vector production system or methods or other aspects of the present invention disclosed herein, the nuclease activity achieved within nuclease helper cell cultures prior to inoculating the main viral vector production vessel is at least about 1000 Benzonase® unit equivalents per mL, preferably wherein said nuclease activity is determinable by the DNAse Alert™ assay provided herein as Assay 1.

In another embodiment of the viral vector production system or methods or other aspects of the present invention disclosed herein, the nuclease activity achieved within nuclease helper cell cultures prior to inoculating the main viral vector production vessel is at least about 1500 Benzonase® unit equivalents per mL, preferably wherein said nuclease activity is determinable by the DNAse Alert™ assay provided herein as Assay 1.

In another embodiment of the viral vector production system or methods or other aspects of the present invention disclosed herein, the nuclease activity achieved within nuclease helper cell cultures prior to inoculating the main viral vector production vessel is at least about 2000 Benzonase® unit equivalents per mL, preferably wherein said nuclease activity is determinable by the DNAse Alert™ assay provided herein as Assay 1.

In another embodiment of the viral vector production system or methods, the nuclease is selected from the group consisting of SmNucA, VsEndA, VcEndA, and BacNucB.

In a further embodiment, the nuclease is Vibrio cholerae Endonuclease I of SEQ ID NO: 1 or variant thereof having at least 90% amino acid identity to SEQ ID NO: 1.

In yet a further embodiment, the nuclease variant is VcEndA variant of any of SEQ ID NOS: 5-11.

In a further embodiment, the nuclease comprises a salt-active nuclease.

In a further embodiment, the salt-active nuclease is Vibrio salmonicida Endonuclease I of SEQ ID NO: 2 or a variant thereof having at least 90% amino acid identity to SEQ ID NO: 2.

In a further embodiment, the nuclease comprises Serratia marcescens Nuclease A of SEQ ID NO: 3 or a variant thereof having at least 90% sequence identity to SEQ ID NO: 3.

In a further embodiment, the nuclease comprises Bacillus BacNucB of SEQ IS NO: 4 or a variant thereof having at least 90% amino acid identity to SEQ ID NO: 4.

In another embodiment, the nuclease comprises an N-terminal secretory signal.

In another embodiment of the invention disclosed herein, the nucleic acid sequences encoding a nuclease are sequence-optimised to remove potential splice sites and/or unstable elements.

In another embodiment, the nucleic acid sequences encoding a nuclease are codon-optimised for expression in the production cell.

In a further embodiment, the production systems and/or methods comprise one or more additional nucleases.

In a further embodiment, the production systems and/or methods comprise a fusion protein of two or more nucleases or nuclease domains.

In an even further embodiment, the fusion protein comprises an endonuclease and an exonuclease.

In another embodiment of the systems or methods or other aspects of the present invention, the cell culture comprises a volume of at least about 5 litres (L) of medium.

In another embodiment of the systems or methods or other aspects of the present invention, the cell culture comprises a volume of at least about 10 litres (L) of medium.

In another embodiment of the systems or methods or other aspects of the present invention, the cell culture comprises a volume of at least about 15 litres (L) of medium.

In another embodiment of the systems or methods or other aspects of the present invention, the cell culture comprises a volume of at least about 20 litres (L) of medium.

In another embodiment of the systems or methods or other aspects of the present invention, the cell culture comprises a volume of at least about 25 litres (L) of medium.

In another embodiment of the systems or methods or other aspects of the present invention, the cell culture comprises a volume of at least about 30 litres (L) of medium.

In another embodiment of the systems or methods or other aspects of the present invention, the cell culture comprises a volume of at least about 35 litres (L) of medium.

In another embodiment of the systems or methods or other aspects of the present invention, the cell culture comprises a volume of at least about 40 litres (L) of medium.

In another embodiment of the systems or methods or other aspects of the present invention, the cell culture comprises a volume of at least about 45 litres (L) of medium.

In another embodiment of the systems or methods or other aspects of the present invention, the cell culture comprises a volume of at least about 50 litres (L) of medium.

In another embodiment of the systems or methods, the cell culture comprises a volume of at least about 60 litres (L) of medium.

In another embodiment of the systems or methods or other aspects of the present invention, the cell culture comprises a volume of at least about 70 litres (L) of medium.

In another embodiment of the systems or methods or other aspects of the present invention, the cell culture comprises a volume of at least about 80 litres (L) of medium.

In another embodiment of the systems or methods or other aspects of the present invention, the cell culture comprises a volume of at least about 90 litres (L) of medium.

In another embodiment of the systems or methods or other aspects of the present invention, the cell culture comprises a volume of at least about 100 litres (L) of medium.

In another embodiment of the systems or methods or other aspects of the present invention, the cell culture comprises a volume of at least about 200 litres (L) of medium.

In another embodiment of the systems or methods or other aspects of the present invention, the cell culture comprises a volume of at least about 500 litres (L) of medium.

In another embodiment of the systems or methods or other aspects of the present invention, the cell culture comprises a volume of up to, or at least, about 1000 litres (L) of medium.

In another embodiment of the systems or methods or other aspects of the present invention, the cells are adherent.

In another embodiment of the systems or methods or other aspects of the present invention, the cells are in suspension.

In another embodiment of the systems or methods or other aspects of the present invention, the cells are adherent in suspension.

In another embodiment of the systems or methods or other aspects of the present invention, the cell culture comprises serum.

In another embodiment of the systems or methods or other aspects of the present invention, the cell culture is serum-free.

In another embodiment of the systems or methods or other aspects of the present invention, the host cell is a mammalian host cell.

In another embodiment of the systems or methods or other aspects of the present invention, the host cell is a human host cell.

In another embodiment of the systems or methods or other aspects of the present invention, the human host cell is a HEK293 cell, or a derivative thereof. Examples of HEK293 derivatives include HEK293S, HEK293SG, HEK293SGGD, HEK293FTM and HEK293T. These derivatives may also be referred to as variants of HEK293.

In one embodiment of the systems or methods or other aspects of the present invention, the HEK293 cell is a HEK293T cell.

In another embodiment of the systems or methods or other aspects of the present invention, the viral vector components comprise a nucleotide of interest (NOI).

In another embodiment of the systems or methods or other aspects of the present invention, the viral vector components are retroviral vector components.

In another embodiment of the systems or methods or other aspects of the present invention, the retroviral vector components are lentiviral vector components.

In another embodiment of the systems or methods or other aspects of the present invention, the viral vector components comprise i) gag-pol; ii) env; iii) optionally the RNA genome of a retroviral vector; and iv) optionally rev, or a functional substitute thereof.

In another embodiment of the systems or methods or other aspects of the present invention, wherein at least two of the nucleic acid sequences encoding the viral vector components are located at the same genetic locus.

In another embodiment of the systems or methods or other aspects of the present invention, at least two of the nucleic acid sequences encoding the viral vector components are in reverse and/or alternating orientations.

In another embodiment of the systems or methods or other aspects of the present invention, at least two of the nucleic acid sequences encoding gag-pol and/or env are associated with at least one regulator element.

In another embodiment of the systems or methods or other aspects of the present invention, the env is a VSV-G env.

In another embodiment of the systems or methods or other aspects of the present invention, the viral vector is an adenoviral vector In another embodiment of the systems or methods or other aspects of the present invention, the viral vector is an adeno-associated viral vector.

In another embodiment of the systems or methods or other aspects of the present invention, the nuclease is expressed and secreted from the cell after transient transfection.

In another embodiment of the systems or methods or other aspects of the present invention, the nuclease is expressed and secreted from the cell after stable integration of the cell.

In another embodiment of the systems or methods or other aspects of the present invention, the expression of the nuclease is inducible or conditional, and wherein the nucleic acid encoding the nuclease comprises an inducible or conditional promoter or regulatory element.

In another embodiment of the systems or methods or other aspects of the present invention, the nuclease is an extracellular nuclease.

In an additional embodiment, the invention disclosed herein provides a transient or stable production cell for producing viral vectors comprising nucleic acid sequences encoding: 1) viral vector components; and 2) a nuclease, wherein the nuclease is expressed in the production cell and secreted in cell culture thereby degrading residual DNA during viral vector production.

In another embodiment, the invention provides a transient or stable production cell for producing viral vectors comprising nucleic acid sequences encoding: 1) viral vector components; and 2) a nuclease fusion protein, wherein the nuclease fusion protein comprises an exonuclease domain fused to an endonuclease domain, and wherein the nuclease fusion protein is expressed in the viral vector production cell and secreted in cell culture thereby degrading residual nucleic acid during viral vector production, and wherein the production cell is a eukaryotic production cell.

In another embodiment, the invention disclosed herein provides a cell culture device, such as a bioreactor, comprising the viral vector production cells expressing and secreting a nuclease.

In addition, disclosed herein is a variant of a secreted nuclease capable of degrading residual nucleic acid during viral vector production, the variant comprising the amino acid sequence of any of SEQ ID NOS: 5-11. In particular, the variant comprises SEQ ID NO: 7, SEQ ID NO: 10, or SEQ ID NO: 11.

Also disclosed herein is a modified nuclease having increased cell-retention and/or or cell-association that is expressed through the secretory pathway of a eukaryotic cell, the modified nuclease comprising a retention signal at its C-terminus.

In another embodiment, the modified nuclease localizes to the endoplasmic reticulum (ER) and/or the golgi compartments thereby resulting in increased cell retention compared to cell retention of a corresponding unmodified nuclease. In an additional embodiment, the modified nuclease is bound to ER receptors of the ER retention-defective complementation group.

In another embodiment, the retention signal is at its C-terminus of consensus [KRHQSA]-[DENQ]-E-L (SEQ ID NO: 19) or KKXX and in a further embodiment, the retention signal is at its C-terminus of consensus KDEL (SEQ ID NO: 21).

In other embodiments, a eukaryotic cell expresses the modified nuclease and a viral vector production system comprising the cell expressing the modified nuclease.

### DESCRIPTION OF THE FIGURES

FIG. 1A-1B illustrate two concepts of the invention disclosed herein showing secretion of nuclease from eukaryotic cells during production of a viral vector production system. In these concepts, the cells expressing secreted nuclease can be either transiently transfected with the nuclease expression cassette or may be stably integrated into the genomes of the cells and may be optionally inducible. In either concept, the mode of viral vector production may be adherent cell culture or suspension cell culture. FIG. 1A shows a culture of viral vector production cells spiked with nuclease helper cells that are capable of expressing secreted nuclease. FIG. 1B shows a viral vector production system wherein the production cells are capable of expressing secreted nuclease themselves without the use of nuclease helper cells.

FIG. 2 illustrates a work flow step-wise approach for modification of a nuclease for genetic engineering in a production cell for use in the invention disclosed herein.

FIG. 3A-3D illustrate schematic nuclease expression cassettes for use in the invention disclosed herein. FIG. 3A shows, without limitation, an exemplary expression cassette for SmNucA. FIG. 3B shows, without limitation, an exemplary expression cassette for VsEndA and VcEndA. FIG. 3C shows, without limitation, an exemplary expression cassette for BacNucB. FIG. 3D shows, without limitation, an exemplary expression cassette for a regulated secreted nuclease cassette for stable cell development.

FIG. 4A-4C show that post-transfection expression and secretion of a nuclease from HEK293T cells correlated with reduced DNA in culture media. FIG. 4A shows transfected cell lysates immunoblotted for anti-histidine tagged smNucAH6. FIG. 4B shows an immuno-dot blot of transfected cell culture media probing using anti-histidine tagged SmNucAH6. FIG. 4C shows residual DNA analysis within the media.

FIG. 5 shows transient lentiviral vector production in adherent or suspension HEK293T cell culture comparing secreted tagged and untagged nucleases (SmNucA) from expression cassettes versus Benzonase® & untreated vector harvests.

FIG. 6 shows transient lentiviral vector production in adherent HEK293T using SmNucA expression cassettes with the native signal peptide and without, wherein exemplary eukaryotic ER signal peptides replace the native signal.

FIG. 7 shows, without limitation, examples of secreted nuclease (e.g., VsEndA & SmNucA) via expression cassette (1% of total input pDNA) during the generation of lentiviral vectors in adherent HEK293T cell cultures during transient transfection with vector components.

FIG. 8 shows, without limitation, examples of secreted nuclease (e.g., VsEndA & SmNucA) via expression cassette (1% of total input pDNA) during the generation of lentiviral vectors in adherent HEK293T producer cell line cultures by dox induction of vector components, during transient transfection with nuclease plasmid.

FIG. 9A-9B show secreted nuclease during vector production in suspension cultures. FIG. 9A shows, without limitation, examples of secreted nuclease (e.g., VsEndA & SmNucA) via expression cassette (5% of total input pDNA) during the generation of lentiviral vectors in HEK293T cell culture in suspension during transient transfection of vector components. FIG. 9B shows concentrated vector supernatant by SDS-PAGE and immunoblotting using anti-histidine tag antibody to detect secreted nuclease proteins.

FIG. 10A-10D show in silico analysis and, without limitation, exemplary modifications to BacNucB for use in the invention disclosed herein. FIG. 10A shows the primary sequence of BacNucB and transmembrane prediction analysis. FIG. 10B shows analysis of the secretory peptide signal of BacNucB. FIG. 10C (SEQ ID NO(s): 4 and 28) shows analysis of BacNucB regions of alpha-helices, beta-strands and coils, and positions of novel NxS/T sequons engineered into four different variants. FIG. 10D (SEQ ID NO: 29) shows the analysis of the primary sequence of BacNucB with the four NxS/T sequon variant positions, and their predicted use as N-glycan sites.

FIG. 11A-11B show enhancement of clearance of residual plasmid DNA in lentiviral vector production cell cultures by engineered BacNucB. FIG. 11A shows the use of BacNucB variants expression plasmids (5% total pDNA) in suspension, serum-free HEK293T cells during lentiviral vector production via transient transfection to reduce pDNA whilst maintaining high vector titres. FIG. 11B shows concentrated vector supernatant by SDS-PAGE and immunoblotting using anti-histidine tag antibody to detect secreted nuclease proteins.

FIG. 12A-12C show enhancement of clearance of residual plasmid DNA in lentiviral vector production cell cultures by use of the engineered variant ‘VcEndA-1glc’, containing mutated NxS/T sequons. FIG. 12A shows VcEndA variants expression plasmids (5% total pDNA) in suspension, serum-free HEK293T cells during lentiviral vector production via transient transfection, whilst maintaining high vector titres. FIG. 12B shows the degree of residual DNA from vector supernatants via agarose-electrophoresis. FIG. 12C shows concentrated vector supernatant or post-production cell lysates by SDS-PAGE and immunoblotting using anti-histidine tag antibody to detect secreted nuclease proteins.

FIG. 13 shows degradation of residual (plasmid) DNA from lentiviral vector production bioreactors at 0.5 L scale using secreted VsEndA from expression cassette.

FIG. 14A-14B: FIG. 14A shows degradation of residual DNA from lentiviral vector production bioreactors at 5 L scale using VsEndA from expression cassette, and total KanR/TUs through subsequent downstream processing. FIG. 14B shows analysis of concentrated samples from some of the process steps (in A) as analysed by gel electrophoresis (ethidium bromide stain for DNA).

FIG. 15A-15B show degradation of residual DNA in lentiviral vector production cell cultures using a tetR-regulated VsEndA expression cassette during co-transfection. FIG. 15A shows that vector production titers remain high. FIG. 15B shows the degree of residual DNA from vector supernatants via agarose-electrophoresis.

FIG. 16A-16B show degradation of residual DNA in lentiviral vector production cell cultures co-cultured with nuclease helper cells expressing tetR-regulated VsEndA or SmNucA. FIG. 16A shows that vector production titers remain high. FIG. 16B shows the degree of residual DNA from vector supernatants via agarose-electrophoresis.

FIG. 17A-17B (SEQ ID NO: 1 and 2) summarise the NxS/T N-glycan sequon mutants of VcEndA. FIG. 17A displays a protein alignment of VsEndA and VcEndA, highlighting the NxS/T sequons within VcEndA that are potential sites of N-glycosylation when expressed in eukaryotic cells. Only the first N-glycan sequon (position 102NCT) of VcEndA is shared with VsEndA, and so the amino acid sequence at the equivalent positions within VsEndA relating to sequons 2, 3 and 4 of VcEndA provide a useful guide for mutation of these sequons. Accordingly, variants of VcEndA were generated (displayed in FIG. 17B; [constructs 9, 19-25]) containing a combination of modifications comprising 119NLT>NLV, and/or 130NRS>DRS and/or 133NFS>NFR, resulting is SEQ ID NOs 1, 5-11.

FIG. 18A-18B show the expression of VcEndAH6 variants relative to SmNucAH6 and VsEndAH6 in post-production cell lysates and culture media in serum free, suspension mode LV production cultures. FIG. 18A displays an immunoblot of LV post-production cell lysates probed against tubulin (loading control) and anti-His6 (SEQ ID NO:27) (nucleases). FIG. 18B displays an immunoblot of concentrated crude vector harvest probed against VSVG (secretion control) and anti-His6 (SEQ ID NO:27) (nucleases). The molecular weights of the nucleases is modulated by N-glycan status (typically a single glycan adds ˜2.5 kDa); the unmodified sizes of the nucleases are: VcEndAH6—27.6 kDa; VsEndAH6—27.9 kDa; SmNucAH6—29.7 kDa. Secreted nuclease plasmids were spiked-in at 0.5% or 5% total plasmid DNA at transfection. The VcEndAH6 variants are described in FIG. 17A-17B, and SEQ ID NOs 1, 5-11.

FIG. 19A-19B display the LV-CMV-GFP vector titres produced in the presence of secreted nucleases described in FIG. 17 and FIG. 18A-18B, as well as the resulting impact on residual DNA in crude vector harvest. At LV harvest, production cells were removed by centrifugation, and supernatants filtered (0.22 μm) before titration by transduction of HEK293T cells followed by flow cytometry; LV titres were calculated accordingly (FIG. 19A). Further, supernatants were concentrated by centrifugation using 3K cut-off Amicon-15 devices, and 50% of this material loaded onto a 2% agarose gel for residual DNA electrophoresis (FIG. 19B; ethidium bromide stained gel). The data show that LV titres are minimally impacted by secreted nucleases, and that engineering of VcEndA N-glycan sequons results in improved secretion of the nuclease, and enhanced residual DNA clearance.

FIG. 20A-20B display confirmed pH set points (FIG. 20A) and cell viability (FIG. 20B) during evaluation of VcEndAH6-1glc, VsEndAH6 and SmNucAH6 in production of LVs in serum-free, suspension cultures under acidic or alkaline culturing conditions.

FIG. 21A-21B show the expression of VcEndAH6-1glc, SmNucAH6 and VsEndAH6 in post-production cell lysates and culture media in serum free, suspension mode LV production cultures under acid or alkaline culturing condtions. FIG. 21A displays an immunoblot of LV post-production cell lysates probed against tubulin (loading control) and anti-His6 (SEQ ID NO:27) (nucleases). FIG. 21B displays an immunoblot of concentrated crude vector harvest probed against VSVG (secretion control) and anti-His6 (SEQ ID NO:27) (nucleases). The molecular weights of the nucleases is modulated by N-glycan status (typically a single glycan adds ˜2.5 kDa); the unmodified sizes of the nucleases are: VcEndAH6—27.6 kDa; VsEndAH6—27.9 kDa; SmNucAH6—29.7 kDa. Secreted nuclease plasmids were spiked-in at 5% total plasmid DNA at transfection.

FIG. 22A-22B display the LV-CMV-GFP vector titres produced in the presence of secreted nucleases VcEndAH6-1glc, SmNucAH6 and VsEndAH6, as well as the resulting impact on residual DNA in crude vector harvest. At LV harvest, production cells were removed by centrifugation, and supernatants filtered (0.22 μm) before titration by transduction of HEK293T cells followed by flow cytometry; LV titres were calculated accordingly (FIG. 22A). Further, supernatants were concentrated by centrifugation using 3K cut-off Amicon-15 devices, and 50% of this material loaded onto a 2% agarose gel for residual DNA electrophoresis (FIG. 22B; ethidium bromide stained gel). The data show that the VcEndAH6-1glc variant displays improved clearance of residual DNA under acidic compared to VsEndAH6, whilst maintaining high titre LV production.

FIG. 23A-23B display confirmed pH set points (FIG. 23A) and cell viability (FIG. 23B) during evaluation of VcEndAH6-124glc, VcEndAH6-1glc, and VsEndAH6 in production of LVs in serum-free, suspension cultures under acidic or alkaline culturing conditions. Interestingly, these data indicate a potential benefit of VcEndAH6-based nuclease expression on cell viability at the latter stages of LV production (also observed in FIG. 20A-20B).

FIG. 24A-24B display the LV-CMV-GFP vector titres produced in the presence of secreted nucleases VcEndAH6-124glc, VcEndAH6-1glc, and VsEndAH6, as well as the resulting impact on residual DNA in crude vector harvest. At LV harvest, production cells were removed by centrifugation, and supernatants filtered (0.22 μm) before titration by transduction of HEK293T cells followed by flow cytometry; LV titres were calculated accordingly (FIG. 24A). Further, supernatants were concentrated by centrifugation using 3K cut-off Amicon-15 devices, and 50% of this material loaded onto a 2% agarose gel for residual DNA electrophoresis (FIG. 24B; ethidium bromide stained gel). The data show that the VcEndAH6-124glc and VcEndAH6-1glc variants displays improved clearance of residual DNA under acidic compared to VsEndAH6, whilst maintaining high titre LV production.

FIG. 25 (SEQ ID NO: 21) displays a schematic showing how nucleases can be modified in order to retain more of the nuclease within cells for use in production of cell-associated viral vectors such as AAVs and AdVs. For some embodiments of the present invention some, preferably all, of the nucleases should be targeted to the secretory pathway by use of a functional ER signal peptide (SP) encoded on the N-terminus of the protein, which is cleaved by Signal Peptidase (SPase) allowing release into the ER lumen. However, by optionally appending the C-terminus of the nuclease with an ER-retention signal such as the sequence ‘KDEL (SEQ ID NO: 21)’ (reading N-to-C), the modified nuclease will be bound by one of the ‘ER retention-defective [ERD] complementation group’ protein receptors that will allow retention of more nuclease within the vector production cell. For production of cell-associated viral vectors such as AAVs and AdVs, this allows production cells to be isolated away (i.e. filtered, precipitated or centrifuged) from culture media prior to cell lysis whilst enabling the nuclease to remain with cells and to be present at the point of cell lysis in order to degrade the production cell DNA.

FIG. 26A-26B display data from the use of secreted and ER-retained nucleases during the production of scAAV2-GFP vector in suspension, serum-free HEK293T cells. Replicate scAAV2 vector production cultures were set up by transfecting cells with Genome, Repcap2 and helper plasmids, and either 5% (total input pDNA) of the indicated nuclease plasmid or no nuclease (pBlueScript; AAV-NEG). The nucleases vcEndAH6 [wt] and vcEndAH6-1glc were optionally engineered with a C-terminal ER-retention signal (KDEL (SEQ ID NO: 21); see FIG. 25), and compared to smNucAH6. Two days post-transfection, cells were harvested by centrifugation and subject to lysis. To the lysate magnesium chloride was added and incubated for 1 hour; for the positive control, SAN was added to AAV-NEG lysate and incubated in parallel. Debris was pelleted by centrifugation, supernatant filtered and analysed directly for vector titre on HEK293T cells [FIG. 26A] or loaded onto an agarose gel to assess residual DNA degradation [FIG. 26B]. Without wishing to be bound by theory, the level of residual DNA within the ‘AAV-NEG’ sample lane may be lower than actually present within untreated cell lysate because much of the released DNA was lost during sample filtration, which was difficult to perform due to viscosity. Therefore, whilst the vcEndAH6 [wt] variant appears to have a lower impact on residual DNA compared to other nuclease variants, it in fact is likely to have substantial activity.

FIG. 27 displays densitometry analysis of the residual DNA agarose gel image presented in FIG. 26B. Using ImageLab, the gel image was divided into multiple channels such that each sample lane was overlaid by 2-3 channels. A representative channel for the stated sample lanes is presented, indicating relative band intensity (arbitrary unit) compared to an empty lane (Background). The resulting band ‘profiles’ show the residual DNA within the gel well and within the sample lane. As can be seen from the image of the gel in FIG. 26B, the short, DNAse resistant forms can be seen, and a correlative peak can be observed within the lane profiles. The vcEndAH6-1glc-KDEL (SEQ ID NO: 21) variant is able to achieve greater clearance of residual DNA over the other variants and SAN.

FIG. 28A-28B display the results of a transfection optimization experiment to identify conditions that achieve close-to-maximal levels of nuclease expression within nuclease helper cell cultures. Transfections of serum-free, suspension HEK293T cells were mediated by mixing lipofectamine 2000CD (at the same ratio per mass of plasmid DNA at each condition) and nuclease encoding plasmid, at 100, 300, 500, 700 or 900 ng per mL of final transfected cell culture. VcEndAH6-1glc or SmNucAH6 expression plasmids driven by a strong CMV or weaker SV40 promoter were used. As a comparison, replicate cultures were also co-transfected with each of the nuclease plasmids at 5% total plasmid input with 95% input of pBluescript, to mimic the type of transfection mix when co-transfecting cells for viral vector production (i.e. nuclease and vector made in the same cell); this was effectively 62 ng of nuclease plasmid per mL. Replicate transfections were induced by sodium butyrate (10 mM final concentration) ˜20 hour post-transfection to mimic conditions within a viral vector production culture. Two days post-transfection, helper cell culture supernatants were filtered (0.2 μm) and concentrated by 3K cut-off spin columns, and cell lysates generated prior to immunoblotting for the nucleases (anti-HisTag), and GAPDH (cell lysates only). [A] Normalised immunoblot band intensities plotted for all conditions. [B] Immunoblots of secreted nuclease protein within helper cells cultures.

FIG. 29A-29C display the results of lentiviral vector (LV-CMV-GFP) production in serum-free, suspension HEK293T cultures at 40 mL shake flask scale using nuclease helper cells generated by transient transfection in parallel to the main vector production culture. Nuclease helper cells were generated by transfection of HEK293T cells with 750 ng (per mL of final culture) of pSV40-VcEndAH6-1glc or pSV40-SmNucAH6 and in parallel to transfection of HEK293T cells with vector components under conditions used in previous Examples. Approximately 20 hours post-transfection, cultures were counted and helper cells added to the main vector production vessel to achieve the desired target percentage proportion of total cells in the main vector production vessel. In this Example, helper cell proportion ranged from 1% to 10% total cells. Sodium butyrate was added at the same time to a final concentration of 10 mM. Two days post-transfection, vector supernatants were harvested, clarified (0.2 μm) and subjected to further analysis. [A] Vector supernatants were titrated by transduction of adherent HEK293T cells followed by FACS two days post-transduction. [B] helper cell and vector supernatants were concentrated by 3K cut-off spin columns and analysed for nuclease expression (anti-HisTag), or for residual DNA analysis on an agarose gel (Ethidium bromide; vector supernatants only) [C].

FIG. 30A-30B display the analysis of production of lentiviral vectors (LV-CMV-GFP) in serum-free, suspension HEK293T cells when using nuclease helper cells to clear residual DNA. In parallel to the main vector production cultures, serum-free suspension HEK293T cells were transfected with pCMV-VcEndAH6-1glc at 750 ng per mL culture media. At the point of sodium butyrate induction, nuclease helper cells were inoculated into the main vector production vessel at the indicated proportion (percentage total cells). Standard vector production continued until two days post-transfection, when vector supernatants were taken for titration on adherent HEK293T cells followed by FACS [A], and also concentrated by 3K cut-off spin columns for agarose gel analysis (ethidium bromide) [B].

FIG. 31A-31B display the correlation between achievable nuclease secretion levels and the specific nuclease activity within lentiviral vector production culture media, when spiking-in nuclease helper cells; the vector preps produce are the same as those in FIG. 30A-30B. Culture supernatants were analysed for nuclease activity by DNAse Alert assay, using Benzonase® as a standard curve [A]. Concentrated culture supernatants from the experiment were analysed by immunoblotting to nuclease (anti-HisTag) [B].

FIG. 32A-32B present the results of lentiviral vector (LV-CMV-GFP) production in serum-free, suspension HEK293T cultures at 5 L bioreactor scale using VcEndAH6-1glc in co-transfection. pSV40-VcEndAH6-1glc was mixed with LV component plasmids at 5% total input, whereas the ‘standard’ control bioreactor was transfected with pBlueScript at 5% total input. Production of vectors proceeded as explained in Example 21, maintaining the pH at 6.7 post-transfection [A]. Approximately 2.5 L of clarified harvest material was taken through the downstream process, with sampling at each stated stage: In-bio (prior to 1 hour incubation with MgCl₂+/−Benzonase®), CLH (clarified harvest; post-treatment with MgCl₂+/−Benzonase®), IEX Eluate (Eluted material from IEX column [diluted to 0.6M NaCl], HFF Pre (buffer exchanged IEX Eluate by dia-/ultra-filtration using hollow fibre cartridge prior to Benzonase® addition), HFF Post (post-Benzonase®-treated, buffer exchanged). Samples were concentrated by 3K cut-off spin columns before loading onto a 2% agar gel containing ethidium bromide [B].

FIG. 33 displays a stained SDS-PAGE gel loaded with cell lysates of serum-free, suspension HEK293T cells transfected with pSV40-VcEndAH6-1glc, pSV40-VcEndAH6-1glc-KDEL (SEQ ID NO: 21) or untransfected cells. VcEndAH6-1glc-KDEL (SEQ ID NO: 21) was enriched within cell extracts compared to VcEndAH6-1glc.

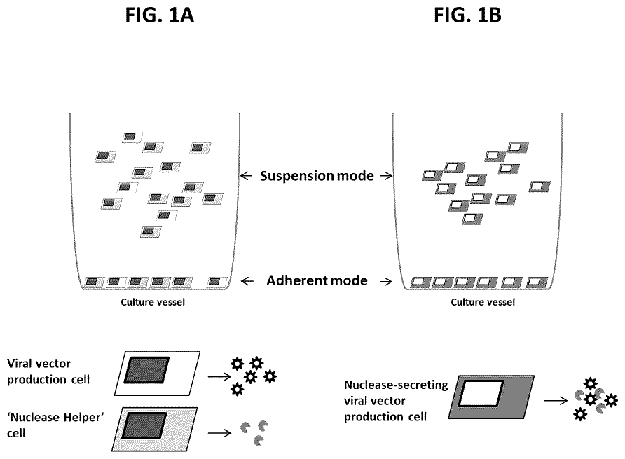

### DETAILED DESCRIPTION OF THE INVENTION

The practice of the invention disclosed herein will employ, unless otherwise indicated, conventional techniques of chemistry, molecular biology, microbiology and immunology, which are within the capabilities of a person of ordinary skill in the art. Such techniques are explained in the literature. See, for example, J. Sambrook, E. F. Fritsch, and T. Maniatis (1989) Molecular Cloning: A Laboratory Manual, Second Edition, Books 1-3, Cold Spring Harbor Laboratory Press; Ausubel, F. M. et al. (1995 and periodic supplements) Current Protocols in Molecular Biology, Ch. 9, 13, and 16, John Wiley & Sons, New York, NY; B. Roe, J. Crabtree, and A. Kahn (1996) DNA Isolation and Sequencing: Essential Techniques, John Wiley & Sons; J. M. Polak and James O'D. McGee (1990) In Situ Hybridization: Principles and Practice; Oxford University Press; M. J. Gait (ed.) (1984) Oligonucleotide Synthesis: A Practical Approach, IRL Press; and, D. M. J. Lilley and J. E. Dahlberg (1992) Methods of Enzymology: DNA Structure Part A: Synthesis and Physical Analysis of DNA Methods in Enzymology, Academic Press. Each of these general texts is herein incorporated by reference.

Unless defined otherwise, all technical and scientific terms used herein have the same meaning as is commonly understood by one of skill in the art to which this invention belongs. Unless otherwise specified, “rev” and “gag-pol” refer to the proteins and/or genes of lentiviral vectors.

### Nuclease-Secreting Viral Vector Production System

The invention disclosed herein is a nuclease-secreting viral vector production system comprising a set of nucleic acid sequences encoding the components required for production of the viral vector and expression and secretion of at least one nuclease, thereby degrading unwanted/contaminating i.e., residual, nucleic acid during viral vector production.

Accordingly, the invention disclosed herein provides an improved vector production system which expresses and secretes a nuclease by viral vector production cells during the production of viral vectors or virus-based vaccines. As a result, unwanted (contaminating), residual nucleic acid (e.g., pDNA) otherwise associated with the crude harvest material is degraded during the production process in a streamlined manner rather than what is typically required with burdensome upstream and/or downstream commercial nuclease enzymatic treatment steps which can sometimes impair to some extent produced viral vector quality and quantity. See well known upstream and downstream processing steps in Merten, O-W., Schweizer, M., Chahal, P., & Kamen, A. A. Manufacturing of viral vectors for gene therapy: part I. Upstream processing. Pharmaceutical Bioprocessing 2, 183-203 (2014); Merten, O-W., Schweizer, M., Chahal, P., & Kamen, A. A. Manufacturing of viral vectors: part II. Downstream processing and safety aspects Pharmaceutical Bioprocessing 2, 237-251 (2014); Gousseinov, E., Kools, W., and Pattnaik, P. Nucleic Acid Impurity Reduction in Viral Vaccine Manufacturing. BioProcess International 12, 59-68 (2014). For the same reasons, the invention disclosed herein is also useful in providing improved exosome and gesicle production systems.

The nucleic acid sequence(s) encoding the nuclease of the invention disclosed herein may be in the form of a nucleotide expression cassette(s) or plasmid. Such an expression cassette(s) is either co-transfected with the viral vector component expression cassette(s) and/or is stably integrated within the viral vector production cell DNA (or cell genome).

In one embodiment, the nucleic acid sequence(s) of the invention disclosed herein is an expression construct encoding one or more nucleases. In another embodiment, the nucleases may be expressed as a fusion protein in such a manner so that two nucleases (or nuclease domains) are secreted during viral vector production. In a further embodiment, the nuclease fusion protein comprises an endonuclease domain and an exonuclease domain, whereby, during viral vector production, the secreted endonuclease degrades circular, plasmid DNA whilst the secreted exonuclease targets and degrades linear DNA from 5′ and/or 3′ termini in order to more efficiently degrade DNA wrapped around the histone proteins, that may be otherwise inaccessible to endonucleases.

The nuclease expression cassette comprises a promoter sequence and/or a regulatory element(s) which drive expression of the nuclease. Such promoters may drive constitutive, inducible, and/or conditional expression of the nuclease. In addition, the nuclease comprises a native and/or a non-native N-terminal secretory signal for secretion of the nuclease into the cell culture.

In an alternative embodiment of the invention disclosed herein, the nuclease expression cassette is used to transiently transfect or stably integrate within a nuclease helper cell used in conjunction (or co-culture) with a viral vector production cell. Such a nuclease helper cell is then spiked-in with viral vector production cells during viral vector manufacture and co-cultured until the end of viral vector production.

The approach of using cultured cells of the invention disclosed herein to secrete a nuclease during the upstream viral vector production process has the advantages of 1) immediate degradation of unwanted or contaminating (residual) nucleic acid within the culture media as soon as viral vector virions are being produced, 2) perpetual secretion of the nuclease throughout viral production without the need for further burdensome manipulations, 3) removing the step of treatment of viral vector crude harvest material with commercial nuclease (e.g., Benzonase®), 4) substantially reduced residual DNA within purified vector product, and 5) lower the cost associated with adding recombinant nuclease during downstream viral vector purification/processing.

The invention disclosed herein provides viral vector production systems (in both transient and stable vector production aspects) expressing secreted nucleases in adherent and/or in suspension, serum-free cell culture. Surprisingly, considering the complex biology involved in the assembly of viral vector virions during viral vector production, the inventors show herein that expression and secretion of a nuclease in cell culture during viral vector production overcomes numerous issues in upstream and downstream viral vector manufacturing whilst still maintaining high production titres. The inventors also demonstrate herein scalability from lab scale or small scale viral vector production to viral vector production in bioreactors.

The invention disclosed herein provides evidence that a secreted nuclease degrades residual DNA within crude vector harvests with improved performance over commercially available nucleases such as Benzonase® and SAN (salt-active nuclease). The invention disclosed herein shows that widely divergent nucleases (e.g., derived from gram-negative and positive bacteria) degrades residual DNA during viral vector production. Because nuclease-appended C-terminal histidine tags are shown herein to not impact functionality of nuclease secretion, anti-histidine-tag based ELISAs were used to measure residual nuclease in final viral vector formulations.

Importantly, the invention disclosed herein provides evidence that the timing of nuclease secretion is temporally linked to gene expression from co-transfected viral vector component expression cassettes. In this manner, nuclease expression initiates at the earliest point at which residual plasmid DNA degradation is desired. Moreover, in another embodiment, inducible expression of nuclease allows for the temporal control of nuclease expression in the viral vector production system disclosed herein.

In one aspect of the invention, a viral vector production cell line is transfected with viral vector expression cassette(s) and an inducible nuclease expression cassette, such as is based on a tetracycline-based approach, which allows for stable secretion of the nuclease into the cell culture media upon addition of the inducer during viral vector production.

In another aspect of the invention, the nuclease cassette is introduced into a viral vector production cell stably expressing viral vector components. In this manner, stable secretion of the nuclease can be constitutive secretion or inducible secretion into the cell culture media depending on the regulatory elements of choice. In the aspect where some of the vector components are also regulated, the nuclease cassette may be under control of the same or separate regulatory elements of the viral vector components, the latter separating temporal control of the nuclease and the vector components.

In another aspect of the invention, the nuclease cassette expresses and secretes nuclease in a retroviral or lentiviral vector production system for degradation of residual nucleic acid during vector production.

In an alternative aspect relating to regulation of nuclease expression in a delayed setting, HIV-1 rev or an analogous retrovirus RNA-export protein is used for modulation of nuclease secretion instead or in addition to the use of a transcription regulatory control element(s) to regulate nuclease expression. In this manner, nuclease-encoding mRNA comprises an HIV-1 rev-responsive element (RRE, which is bound by HIV-1 rev) or analogous retrovirus RNA-export protein-responsive element. The nuclease ORF and RRE are placed within the same intron within the expression cassette and therefore in the absence of HIV-1 rev (or analogous retrovirus RNA-export protein) the amount of nuclease-encoding mRNA within the cytoplasm is reduced compared to amounts of nuclease in the presence of rev. HIV-1 rev or the analogous retrovirus RNA-export protein is co-expressed at a desired time point during production of the vector in order to increase expression of the nuclease.

Disclosed herein is evidence that a nuclease helper cell can be introduced during the viral vector production upstream process to co-culture with the viral vector production cells. In this regard, nuclease is supplied in parallel.

In another aspect of the invention, the nuclease-helper cell or helper cell can be cultured in parallel to the viral vector production culture, and then media comprising secreted nuclease is fed to the viral vector production cell culture.

In another aspect, the nuclease helper cell or helper cell can be cultured in parallel to the exosome production culture, and then media comprising secreted nuclease is fed to the exosome production cell culture.

In another aspect, the nuclease helper cell or helper cell can be cultured in parallel to the gesicle production culture, and then media comprising secreted nuclease is fed to the exosome gesicle cell culture.

In another aspect of the invention disclosed herein, the nuclease cassette expresses and secretes nuclease in an AAV or adenoviral vector production system for degradation of residual nucleic acid during vector production.

Typically, AAV-based vectors are produced in mammalian cell lines (e.g. HEK293-based) or through use of the baculovirus/Sf9 insect cell system. AAV vectors can be produced by transient transfection of vector component encoding DNAs, typically together with helper functions from Adenovirus or Herpes Simplex virus (HSV), or by use of cell lines stably expressing AAV vector components. Adenoviral vectors are typically produced in mammalian cell lines that stably express Adenovirus E1 functions (e.g. HEK293-based). Adenoviral vectors are also typically ‘amplified’ via helper-function-dependent replication through serial rounds of ‘infection’ using the production cell line. For the invention disclosed herein, the common feature of AAV vector and Adenovirus vector production is the step of cell lysis in order to more efficiently release vector virions that are cell-associated. Some methods, such as freeze-thaw, allow for cell lysis within the harvest media, whilst at larger scales it may be desirable to concentrate production cells (e.g. by centrifugation or filtration) before cell lysis by freeze-thaw or mechanical or chemical treatment. At the cell lysis step, the crude vector typically becomes substantially contaminated with production cell DNA, and usually a commercial source of recombinant nuclease protein (such as Benzonase® or SAN-HQ) is used either immediately prior to and/or subsequently to cell lysis in order to minimise the amount of DNA; this reduces viscosity and facilitates subsequent downstream processing.

Therefore, the invention disclosed herein includes, without limitation, the use of secreted nuclease in place of commercial nuclease in AAV and Adenovirus vector production systems and manufacturing processes. In the context of such systems and processes, the secreted nuclease is present within either the media (if cell lysis is occurring within the culture media) and/or within the vector production cells as a ‘steady-state’ pool of protein, since the nuclease is continually expressed and secreted into the ER-Golgi network as a mature nuclease. Nuclease present within the production cells is present if cells are concentrated-away from culture media but is released upon cell lysis, and therefore has access to contaminating (or residual) DNA. Alternatively, clarified culture supernatant comprising the secreted nuclease as well as ‘free’ vector virions may be ‘added-back’ to the bulk cell lysate to cause DNA degradation prior to further downstream processing. In one aspect of the invention, nuclease secretion in such AAV- or Adenovirus-based vector production (or indeed any viral vector platform wherein vector virions remain chiefly cell associated) where cell lysis is required, may utilise a eukaryotic cell line containing an inducible, stable nuclease expression cassette. In another aspect, nuclease secretion may be achieved via transient transfection of the nuclease plasmid (with other vector components) for AAV vector production or production of Adenovirus vector master seed stock when performing recombination within transfected cells (e.g. RapAd® system).

### Nucleases

Nuclease structure and function is well known in the field (Yang, Q. Rev. Biophys. 2011 February; 44(1)L1-93). Nucleases are a ubiquitous class of enzymes that hydrolyse nucleic acids—DNA and RNA. These enzymes mediate the hydrolysis of the phosphodiester bond within the backbone of polynucleotides resulting in cleavage. Many nucleases require metal ions for their maximal activity. Nucleases can be categorised into two broad groups governed by their mode off attack: [1] Endonucleases and [2] Exonucleases.

Endonucleases attack the phosphodiester bond within the polynucleotide chain (interior/‘endo’); some endonucleases are non-specific meaning that they cleave between any nucleotide, whilst others may have site preference at specific di-nucleotides.

Exonucleases digest from the termini of the nucleic acids present at either 5′ or 3′ ends of the polynucleotide chains. Some nucleases, for example Nuclease Bal-31, may have both endo- and exon-nuclease properties, being able to both digest DNA from termini and within the polynucleotide, depending on the nature of the target (i.e. whether the target is dsDNA or ssDNA).

Given the lack of requirement for the target polynucleotide to possess free 5′ or 3′ termini, endonucleases are able to digest circular forms of DNA (such as plasmid DNA). As such, a nuclease of the invention disclosed herein is a nuclease from the ‘sugar-non-specific’ nucleases, since these enzymes can non-specifically degrade ssDNA, dsDNA, ssRNA and/or dsRNA, in a sequence-independent manner. This group includes the ‘Serratia’ family of nucleases, which includes bacteria nucleases (NucA) and eukaryotic mitochondrial endonuclease G (Endo G), which is also known as Nuc1p in yeast. The Serriatia nucleases are structurally very similar; the catalytic ββα motif forms a structural subdomain and is packed against one face of a six-stranded antiparallel β-sheet. The ‘sugar-non-specific’ nucleases also include the bacterial periplasmic endonuclease I family of nucleases e.g. VsEndA.

Nucleases secreted from eukaryotic or prokaryotic cells share analogous secretion pathways which are governed by similar features of all secreted proteins of classical secretion pathways. A relatively short peptide (typically less than 30 residues) located at the N-terminus of the pre-mature nuclease protein emerging from the ribosome, is recognised by cellular machinery and physically relocated from the cytoplasm/cytosol to the membrane of the endoplasmic reticulum (ER) or plasma membrane of eukaryotes or prokaryotes, respectively, where translation continues through the membrane into a separate cellular compartment. After protein translation, the secretory peptide is cleaved where protein folding is completed and the mature nuclease is contained within the ER (eukaryotes) or periplasm (prokaryotes). The nuclease will then typically be released into the extracellular space.

The invention disclosed herein includes, without limitation, the use of wild-type and modified or mutated (i.e., variant) nucleases capable of being secreted in viral vector production. Such nucleases may optionally include eukaryotic secretory signals to replace bacterial or native secretory peptides. Exemplary secretory signal peptides include, without limitation, those listed in the following table:

| | | |
|---|---|---|
| Secrecon | MWWRLWWLLLLLLLLWPMVWA | Synthetic (Barash et al. Biochem |
| | (SEQ ID NO: 12) | Biophys Res Commun, 2002, |
| | | Jun. 21; 294(4): 835-842.) |
| Mouse IgKVIII | METDTLLLWVLLLWVPGSTG | Mouse Ig kappa |
| | (SEQ ID NO: 13) | |
| Human 1gKVIII | MDMRVPAQLLGLLLLWLRGARC | Human Ig kappa |
| | (SEQ ID NO: 14) | |
| CD33 | MPLLLLLPLLWAGALA | Human CD3 |
| | (SEQ ID NO: 15) | |
| tPA | MDAMKRGLCCVLLLCGAVFVSPS | Human tPA |
| | (SEQ ID NO: 16) | |
| Albumin | MKWVTFISLLFSSAYS | Human serum albumin |
| | (SEQ ID NO: 17) | |
| VSVG | MKCLLYLAFLFIGVNC | VSV Glycoprotein |
| | (SEQ ID NO: 18) | |

Nucleases for use in the invention disclosed herein may include modifications and/or mutations relating to function-disrupting N-glycan sequons or creation of novel N-glycan sequon sites. Secretion of a protein with nuclease activity that can be efficiently secreted from a eukaryotic cell through the classical secretory pathway necessitates the presence of a functional secretory signal-peptide encoded at the beginning of the open-reading frame (ORF), and often the secreted protein will comprise at least one asparagine-linked (N-linked) glycan so that secretion is made more efficient. Glycosylation occurs at the asparagine residue of NxS or NxT sequons (where x can be any amino acid except proline) during export through the secretory pathway found in eukaryotic cells, bacterial secreted nucleases are not glycosylated in this manner. However, since the NxS/T motif also occurs often in bacterial nucleases, these sites in principle could be utilized if bacterial nucleases are expressed from eukaryotic cells, as per the invention disclosed herein. N-glycan site predictor programs (such as NetNGlyc) can be helpful in identifying which NxS/T site might be most likely to be utilized. Additionally, determination can be made as to the proximity of N-glycan to the DNAse active site and any impact on DNA activity. Indeed, the inventors show that nucleases can be mutated to modulate or modify NxS/T sequons so as to improve secretion in the context of the viral vector production system and/or per the production cells as disclosed herein.

A nuclease of the invention disclosed herein may include modification of a C-terminal appendage of a nuclease with a retention signal, such as an ER retention signal, which improves cell-retention or cell-association by localising the nuclease to the ER and/or golgi compartments. In this manner, the nuclease binds to the ER receptors of the ER retention-defective complementation group. Such a modification improves residual DNA clearance from cell lysate during vector production, particularly during AAV vector production. Retention signals include, without limitation, a retention signal of consensus [KRHQSA]-[DENQ]-E-L (SEQ ID NO: 19) or KKXX, and/or a retention signal of consensus KDEL (SEQ ID NO: 21). C-terminal retention signals having KDEL (SEQ ID NO: 21) motifs are known in the art (e.g., Raykhel et al., J. Cell. Biol., 2007 Dec. 7, 179(6): 1193-1204).

Alternative modifications to the nucleases of the invention for achieving cell-retention/association include, without limitation, use of a GPI anchor or transmembrane domain at the C-terminus of the nuclease. As such, in alternative embodiments, a modified nuclease comprises a GPI anchor and/or a transmembrane domain at its C-terminus.

A nuclease of the invention disclosed herein, without limitation, is a nuclease with an optimal pH or pH range for functionality in a viral vector production system. Secreted nucleases of the invention function at an optimal pH or optimal pH range for the degradation of residual nucleic acid during viral vector production. Optimal pH of a secreted nuclease of the invention disclosed herein is, without limitation, about pH6, about pH6.5, about pH6.6, about pH6.8, about pH7, about pH7.2, about pH7.5. An optimal pH range of a secreted nuclease of the invention is, without limitation, about pH6-about pH7, about pH6-about pH6.5, about pH6-about pH7.2, about pH6.5-about pH7.2, about pH6.5-about pH7.5, about pH6.5-about pH7, about pH6.5-about pH6.8, about pH6.6-about pH7.2, about pH6.6-about pH7.5, about pH6.6-about pH7.2, about pH6.6-about pH7, about pH6.6-about pH6.8, about pH6.8-about pH7, about pH6.8-about pH7.2, about pH7-about pH7.2, about pH7-about pH7.5, about pH7.2-about pH7.5.

A nuclease of the invention disclosed herein is, without limitation, a hydrolytic nuclease capable of being secreted, such as, without limitation, an extracellular nuclease or a nuclease modified to function as an extracellular nuclease, in a viral vector production system.

Widespread use of commercial nuclease (protein-form) occurs in a manufacturing context when there is a need to remove as much as possible of the contaminating (or residual) nucleic acids (both DNA and RNA) from a production system. It is well known that the requirement to remove nucleic acids is particularly important if production occurs intracellularly or if cells are lysed during production. As a result of this, large amounts of contaminating nucleic acids are released which make further purification processes, such as filtration and/or chromatography, more challenging due to, e.g., increased viscosity of the sample.

As such, a nuclease of the invention disclosed herein is, without limitation, a nuclease that cleaves (i.e., degrades) contaminating (or unwanted) residual nucleic acid (preferably DNA but may also have RNAse activity) whilst exhibiting nuclease functionality and stability in the context of a viral vector production system.

A nuclease of the invention disclosed herein is, without limitation, a prokaryotic nuclease, a eukaryotic nuclease, and/or a functional variant or derivative thereof that is capable of degrading residual nucleic acid in a viral vector production system.

A nuclease of the invention disclosed herein is, without limitation, a sugar-non-specific nuclease, and/or a functional variant, domain, or derivative thereof that is capable of degrading dsDNA, ssDNA, dsRNA and/or ssRNA in a viral vector production system.

A nuclease of the invention herein is, without limitation, a nuclease derived from an organism selected from the group consisting of Vibrio cholerae, Vibrio salmonicida, Serratia marcescens, and Bacillus licheniformis, variants thereof, and combinations thereof.

A nuclease of the invention herein is, without limitation, any known nuclease or nuclease derived from a known organism. (See Wang, supra.) For example, without limitation, a nuclease of the invention is a nuclease derived from Vibrio cholerae, Vibrio salmonicida, Serratia marcescens, and Bacillus licheniformis. Furthermore, without limitation, a nuclease of the invention is any known nuclease in the following categories:
Predatory bacterial nucleases e.g. Bdellovibrio bacteriovorus Bd1244, Bd1934
Plant nucleases e.g. Hordeum vulgare L microspore nuclease (Barley)
Snase family nucleases e.g. Staphylococcus aureus NucA
Intestinal nucleases e.g. Pancreatic DNAse I
Low pH active e.g. Mycobacterium smegmatis Rv0888 (low pH nuclease)

A nuclease of the invention disclosed herein is, without limitation, selected from the group consisting of smNucA (NCBI Reference Sequence: WP_047571650.1), VsEndA (GenBank: CAQ78235.1), VcEndA (NCBI Reference Sequence: WP_000972597.1), BacNucB (NCBI Reference Sequence: WP_003182220.1), variants thereof, and combinations thereof.

A nuclease of the invention disclosed herein is, without limitation, a modified variant of Endonuclease I from Vibrio cholerae (herein referred to as VcEndA). The reference sequence for wild-type VcEndA [Predicted eukaryotic signal peptide in bold, NxS/T sequons underlined] is provided herein as SEQ ID NO: 1:

MMIFRFVTTLAASLPLLTFAAPISESHAKNEAVKIYRDHPVSFYCGCEIR
WQGKKGIPDLESCGYQVRKNENRASRIEWEHVVPAWQFGHQLQCWQQGGR
KNCTRTSPEFNQMEADLHNLTPAIGEVNGNRSNFSFSQWNGIDGVTYGQC
EMQVNFKERTAMPPERARGAIARTYLYMSEQYGLRLSKAQNQLMQAWNNQ
YPVSEWECVRDQKIEKVQGNSNRFVREQCPN

Without limitation, variants of VcEndA with referenced mutations are as follows:

SEQ ID NO: 5 ‘VcEndA-12glc’:
MMIFRFVTTLAASLPLLTFAAPISFSHAKNEAVKIYRDHPVSFYCGCEIR
WQGKKGIPDLESCGYQVRKNENRASRIEWEHVVPAWQFGHQLQCWQQGGR
KNCTRTSPEFNQMEADLHNLTPAIGEVNGDRSNFRFSQWNGIDGVTYGQC
EMQVNFKERTAMPPERARGAIARTYLYMSEQYGLRLSKAQNQLMQAWNNQ
YPVSEWECVRDQKIEKVQGNSNRFVREQCPN
SEQ ID NO: 6 ‘VcEndA-123glc’:
MMIFRFVTTLAASLPLLTFAAPISFSHAKNEAVKIYRDHPVSFYCGCEIR
WQGKKGIPDLESCGYQVRKNENRASRIEWEHVVPAWQFGHQLQCWQQGGR
KNCTRTSPEFNQMEADLHNLTPAIGEVNGNRSNFRFSQWNGIDGVTYGQC
EMQVNFKERTAMPPERARGAIARTYLYMSEQYGLRLSKAQNQLMQAWNNQ
YPVSEWECVRDQKIEKVQGNSNRFVREQCPN
SEQ ID NO: 7 ‘VcEndA-124glc’:
MMIFRFVTTLAASLPLLTFAAPISFSHAKNEAVKIYRDHPVSFYCGCEIR
WQGKKGIPDLESCGYQVRKNENRASRIEWEHVVPAWQFGHQLQCWQQGGR
KNCTRTSPEFNQMEADLHNLTPAIGEVNGDRSNFSFSQWNGIDGVTYGQC
EMQVNFKERTAMPPERARGAIARTYLYMSEQYGLRLSKAQNQLMQAWNNQ
YPVSEWECVRDQKIEKVQGNSNRFVREQCPN
SEQ ID NO: 8 ‘VcEndA-134glc’:
MMIFRFVTTLAASLPLLTFAAPISFSHAKNEAVKIYRDHPVSFYCGCEIR
WQGKKGIPDLESCGYQVRKNENRASRIEWEHVVPAWQFGHQLQCWQQGGR
KNCTRTSPEFNQMEADLHNLVPAIGEVNGNRSNFSFSQWNGIDGVTYGQC
EMQVNFKERTAMPPERARGAIARTYLYMSEQYGLRLSKAQNQLMQAWNNQ
YPVSEWECVRDQKIEKVQGNSNRFVREQCPN
SEQ ID NO: 9 ‘VcEndA-13glc’:
MMIFRFVTTLAASLPLLTFAAPISFSHAKNEAVKIYRDHPVSFYCGCEIR
WQGKKGIPDLESCGYQVRKNENRASRIEWEHVVPAWQFGHQLQCWQQGGR
KNCTRTSPEFNQMEADLHNLVPAIGEVNGNRSNFRFSQWNGIDGVTYGQC
EMQVNFKERTAMPPERARGAIARTYLYMSEQYGLRLSKAQNQLMQAWNNQ
YPVSEWECVRDQKIEKVQGNSNRFVREQCPN
SEQ ID NO: 10 ‘VcEndA-14glc’:
MMIFRFVTTLAASLPLLTFAAPISFSHAKNEAVKIYRDHPVSFYCGCEIR
WQGKKGIPDLESCGYQVRKNENRASRIEWEHVVPAWQFGHQLQCWQQGGR
KNCTRTSPEFNQMEADLHNLVPAIGEVNGDRSNFSFSQWNGIDGVTYGQC
EMQVNFKERTAMPPERARGAIARTYLYMSEQYGLRLSKAQNQLMQAWNNQ
YPVSEWECVRDQKIEKVQGNSNRFVREQCPN
SEQ ID NO: 11 ‘VcEndA-1glc’:
MMIFRFVTTLAASLPLLTFAAPISFSHAKNEAVKIYRDHPVSFYCGCEIR
WQGKKGIPDLESCGYQVRKNENRASRIEWEHVVPAWQFGHQLQCWQQGGR
KNCTRTSPEFNQMEADLHNLVPAIGEVNGDRSNFRFSQWNGIDGVTYGQC
EMQVNFKERTAMPPERARGAIARTYLYMSEQYGLRLSKAQNQLMQAWNNQ
YPVSEWECVRDQKIEKVQGNSNRFVREQCPN

VcEndA is a species of curved rod-shaped gram-negative bacteria in the family Vibrionaceae. This bacterial nuclease comprises a bacteria secretory signal that functions as a eukaryotic secretory signal peptide. The optimal salt concentration of this nuclease is 150-200 mM, and it is active over a wide range of pH, making it an ideal candidate extracellular nuclease for secretion into viral vector production cell media. Whilst the unmodified VcEndA has detectable secretion and DNAse activity when expressed from vector production cells, ablation of the NxS/T motifs starting at N¹¹⁹, N¹³⁰, N¹³³ (residue numbers relative to pre-mature native VcEndA) enables improved secretion of VcEndA and clearance of DNA in the invention disclosed herein.

A nuclease of the invention disclosed herein is, without limitation, a ‘salt-active’ nuclease, which is typically derived from a halophilic microbial organism. Unlike other nucleases, salt-active nucleases maintain activity in hyper-physiological salt concentration (e.g. >0.15M [NaCl]). As such, post-harvest clarified vector material and the salt-active nuclease can be incubated with hyper-physiological salt concentration to potentially aid the dissociation of nucleic acid bound to protein within the harvest material, making it more accessible to nuclease cleavage.

A salt-active nuclease of the invention disclosed herein is, without limitation, ‘Endonuclease I’ from Vibrio salmonicida (herein referred to as ‘VsEndA’). See Altermark B1, Niiranen L, Willassen N P, Smalås A O, Moe E. Comparative studies of endonuclease I from cold-adapted Vibrio salmonicida and mesophilic Vibrio cholerae. FEBS J. 274(1):252-63 (2007).

The reference sequence for wild-type VsEndA (predicted eukaryotic signal peptide above in bold, NxS/T sequons underlined) is provided herein as SEQ ID NO: 2:

Endonuclease I [V. Salmonicida](‘VsEndA’)
MKLIRLVISLIAVSFTVNVMAAPPSSESKAKKEAVKIYLDYPTSFYCGCD
ITWKNKKKGIPELESCGYQVRKQEKRASRIEWEHVVPAWQFGHQRQCWQK
GGRKNCTRNDKQFKSMEADLHNLVPAIGEVNGDRSNFRFSQWNGSKGAFY
GQCAFKVDFKGRVAEPPAQSRGAIARTYLYMNNEYKFNLSKAQRQLMEAW
NKQYPVSTWECTRDERIAKIQGNHNQFVYKACTK

VsEndA is a species of curved rod-shaped gram-negative bacteria in the family Vibrionaceae. This bacterial nuclease contains a bacteria secretory signal that functions as a eukaryotic secretory signal peptide.

A nuclease of the invention disclosed herein is, without limitation, ‘Nuclease A’ from Serratia marcescens (hereinafter ‘SmNucA’), which is a species of rod-shaped gram-negative bacteria in the family Enterobacteriaceae. The reference sequence for wild-type SmNucA (predicted eukaryotic signal peptide above in bold, NxS/T sequons underlined) is provided herein as SEQ ID NO: 3:

Nuclease A [S. marcescens](‘SmNucA’)
MRFNNKMLALAALLFAAQASADTLESIDNCAVGCPTGGSSNVSIVRHAYT
LNNNSTTKFANWVAYHITKDTPASGKTRNWKTDPALNPADTLAPADYTGA
NAALKVDRGHQAPLASLAGVSDWESLNYLSNITPQKSDLNQGAWARLEDQ
ERKLIDRADISSVYTVTGPLYERDMGKLPGTQKAHTIPSAYWKVIFINNS
PAVNHYAAFLFDQNTPKGADFCQFRVTVDEIEKRTGLIIWAGLPDDVQAS
LKSKPGVLPELMGCKN

A nuclease of the invention disclosed herein is, without limitation, ‘Nuclease B’ from Bacillus licheniformis, hereinafter ‘BacNucB’, which is a species of rod-shaped gram-positive bacteria in the family Enterobacteriaceae. The reference sequence for wild-type BacNucB (predicted eukaryotic signal peptide above in bold) is provided herein as SEQ ID NO: 4:

Endonuclease B [Bacillus sp. E.g. B.
licheniformis](‘BacNucB’)
MIKKWAVHLLFSALVLLGLSGGAAYSPQHAEGAARYDDILYFPASRYPET
GAHISDAIKAGHSDVCTIERSGADKRRQESLKGIPTKPGFDRDEWPMAMC
EEGGKGASVRYVSSSDNRGAGSWVGNRLSGFADGTRILFIVQ

### Vectors

The invention disclosed herein relates to viral vectors and the manufacturing and/or production of the same.

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred into and expressed by a target cell. The vector may facilitate the integration of the nucleic acid/nucleotide of interest (NOI) to maintain the NOI and its expression within the target cell. Alternatively, the vector may facilitate the replication of the vector through expression of the NOI in a transient system.

The vectors of the invention are viral vectors, in particular retroviral vectors, with a promoter for the expression of the said NOI and optionally a regulator of the NOI. The vectors may contain one or more selectable marker genes (e.g. a neomycin resistance gene) and/or traceable marker gene(s) (e.g. a gene encoding green fluorescent protein (GFP)). Vectors may be used, for example, to infect and/or transduce a target cell.

The viral vector may be used to express the NOI in a compatible target cell in vitro. Thus, the invention provides a method of making proteins in vitro by introducing a vector of the invention into a compatible target cell in vitro and growing the target cell under conditions which result in expression of the NOI. Protein and NOI may be recovered from the target cell by methods well known in the art. Suitable target cells include mammalian cell lines and other eukaryotic cell lines.

The vector may be an expression vector. Expression vectors as described herein comprise regions of nucleic acid containing sequences capable of being transcribed. Thus, sequences encoding mRNA, tRNA and rRNA are included within this definition.

In some aspects the vectors may have “insulators”—genetic sequences that block the interaction between promoters and enhancers, and act as a barrier reducing read-through from an adjacent gene.

In one embodiment the insulator is present between one or more of the retroviral nucleic acid sequences to prevent promoter interference and read-thorough from adjacent genes. If the insulators are present in the modular construct between one or more of the retroviral nucleic acid sequences, then each of these insulated genes may be arranged as individual expression units.

In one aspect, the invention provides a cell for producing retroviral vectors comprising nucleic acid sequences encoding:
i) gag-pol;
ii) env;
iii) optionally the RNA genome of the retroviral vector; and
iv) optionally rev, or a functional substitute thereof,

In another aspect, vectors of the invention comprise at least two nucleic acid sequences that are located at the same genetic locus; wherein the at least two nucleic acid sequences are in reverse and/or alternating orientations; and wherein the nucleic acid sequences encoding gag-pol and/or env are associated with at least one regulatory element.

In another embodiment, the retrovirus is derived from a foamy virus.

In another embodiment, the retroviral vector is derived from a lentivirus.

In another embodiment, the lentiviral vector is derived from HIV-1, HIV-2, SIV, FIV, BIV, EIAV, CAEV or Visna lentivirus.

### Retroviral and Lentiviral Vectors

The retroviral vector of the invention disclosed herein may be derived from or may be derivable from any suitable retrovirus. A large number of different retroviruses have been identified. Examples include: murine leukemia virus (MLV), human T-cell leukemia virus (HTLV), mouse mammary tumour virus (MMTV), Rous sarcoma virus (RSV), Fujinami sarcoma virus (FuSV), Moloney murine leukemia virus (Mo MLV), FBR murine osteosarcoma virus (FBR MSV), Moloney murine sarcoma virus (Mo-MSV), Abelson murine leukemia virus (A-MLV), Avian myelocytomatosis virus-29 (MC29) and Avian erythroblastosis virus (AEV). A detailed list of retroviruses may be found in Coffin et al. (1997) “Retroviruses”, Cold Spring Harbour Laboratory Press Eds: J M Coffin, SM Hughes, HE Varmus pp 758-763.

Retroviruses may be broadly divided into two categories, namely “simple” and “complex”. Retroviruses may even be further divided into seven groups. Five of these groups represent retroviruses with oncogenic potential. The remaining two groups are the lentiviruses and the spumaviruses. A review of these retroviruses is presented in Coffin et al (1997) ibid.

The basic structure of retroviral and lentiviral genomes share many common features such as a 5′ LTR and a 3′ LTR, between or within which are located a packaging signal to enable the genome to be packaged, a primer binding site, integration sites to enable integration into a target cell genome and gag/pol and env genes encoding the packaging components—these are polypeptides required for the assembly of viral particles. Lentiviruses have additional features, such as the rev gene and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a typical retroviral vector of the invention, at least part of one or more protein coding regions essential for replication may be removed from the virus; for example, gag/pol and env may be absent or not functional. This makes the viral vector replication-defective.

Lentiviruses are part of a larger group of retroviruses. A detailed list of lentiviruses may be found in Coffin et al (1997) “Retroviruses” Cold Spring Harbour Laboratory Press Eds: J M Coffin, SM Hughes, HE Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), feline immunodeficiency virus (FIV), Maedi visna virus (MVV) and bovine immunodeficiency virus (BIV).

The lentivirus family differs from retroviruses in that lentiviruses have the capability to infect both dividing and non-dividing cells (Lewis et al (1992) EMBO J 11(8):3053-3058 and Lewis and Emerman (1994) J Virol 68 (1):510-516). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects or transduces target cells and expresses NOI.

The lentiviral vector may be derived from either a primate lentivirus (e.g. HIV-1) or a non-primate lentivirus (e.g. EIAV).

In general terms, a typical retroviral vector production system involves the separation of the viral genome from the essential viral packaging functions. These vector components are typically provided to the production cells on separate DNA expression cassettes (alternatively known as plasmids, expression plasmids, DNA constructs or expression constructs).

The vector genome comprises the NOI. Vector genomes typically require a packaging signal (ψ), the internal expression cassette harbouring the NOI, (optionally) a post-transcriptional element (PRE), the 3′-ppu and a self-inactivating (SIN) LTR. The R-U5 regions are required for correct polyadenylation of both the vector genome RNA and NOI mRNA, as well as the process of reverse transcription. The vector genome may optionally include an open reading frame, as described in WO 2003/064665, which allows for vector production in the absence of rev.

The packaging functions include the gag/pol and env genes. These are required for the production of vector particles by the production cell. Providing these functions in trans to the genome facilitates the production of replication-defective viral vectors.

Production systems for gamma-retroviral vectors are typically 3-component systems requiring genome, gag/pol and env expression constructs. Production systems for HIV-1-based lentiviral vectors may additionally require the accessory gene rev to be provided and for the vector genome to include the rev-responsive element (RRE). EIAV-based lentiviral vectors do not require rev to be provided in trans if an open-reading frame (ORF) is present within the genome (see WO 2003/064665).

Usually both the “external” promoter (which drives the vector genome cassette) and “internal” promoter (which drives the NOI cassette) encoded within the vector genome cassette are strong eukaryotic or virus promoters, as are those driving the other vector system components. Examples of such promoters include CMV, EF1α, PGK, CAG, TK, SV40 and Ubiquitin promoters. Strong ‘synthetic’ promoters, such as those generated by DNA libraries (e.g. JeT promoter) may also be used to drive transcription. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VMD2), Tyrosine hydroxylase, neuronal-specific neuronal-specific enolase (NSE) promoter, astrocyte-specific glial fibrillary acidic protein (GFAP) promoter, human al-antitrypsin (hAAT) promoter, phosphoenolpyruvate carboxykinase (PEPCK), liver fatty acid binding protein promoter, Flt-1 promoter, INF-β promoter, Mb promoter, SP-B promoter, SYN1 promoter, WASP promoter, SV40/hAlb promoter, SV40/CD43, SV40/CD45, NSE/RU5′ promoter, ICAM-2 promoter, GPIIb promoter, GFAP promoter, Fibronectin promoter, Endoglin promoter, Elastase-1 promoter, Desmin promoter, CD68 promoter, CD14 promoter and B29 promoter may be used to drive transcription.

Production of retroviral vectors involves either the transient co-transfection of the production cells with these DNA components or use of stable production cell lines wherein all the components are stably integrated within the production cell genome (e.g. Stewart H J, Fong-Wong L, Strickland I, Chipchase D, Kelleher M, Stevenson L, Thoree V, McCarthy J, Ralph G S, Mitrophanous K A and Radcliffe P A. (2011). Hum Gene Ther. March; 22 (3):357-69). An alternative approach is to use a stable packaging cell (into which the packaging components are stably integrated) and then transiently transfect in the vector genome plasmid as required (e.g. Stewart, H. J., M. A. Leroux-Carlucci, C. J. Sion, K. A. Mitrophanous and P. A. Radcliffe (2009). Gene Ther. June; 16 (6):805-14). It is also feasible that alternative, not complete, packaging cell lines could be generated (just one or two packaging components are stably integrated into the cell lines) and to generate vector the missing components are transiently transfected. The production cell may also express regulatory proteins such as a member of the tet repressor (TetR) protein group of transcription regulators (e.g. T-Rex, Tet-On, and Tet-Off), a member of the cumate inducible switch system group of transcription regulators (e.g. cumate repressor (CymR) protein), or an RNA-binding protein supplied to repress transgene expression during production (e.g. TRAP—tryptophan-activated RNA-binding protein).

In one embodiment of the invention disclosed herein, the viral vector is derived from EIAV. EIAV has the simplest genomic structure of the lentiviruses. In addition to the gag/pol and env genes, EIAV encodes three other genes: tat, rev, and S2. Tat acts as a transcriptional activator of the viral LTR (Derse and Newbold (1993) Virology 194(2):530-536 and Maury et al (1994) Virology 200(2):632-642) and rev regulates and coordinates the expression of viral genes through rev-response elements (RRE) (Martarano et al. (1994) J Virol 68(5):3102-3111). The mechanisms of action of these two proteins are thought to be broadly similar to the analogous mechanisms in the primate viruses (Martarano et al. (1994) J Virol 68(5):3102-3111). The function of S2 is to antagonize SERINC3/5 (Chande et al., PNAS, 2016 November 15; 113(46):13197-13202. In addition, an EIAV protein, Ttm, has been identified that is encoded by the first exon of tat spliced to the env coding sequence at the start of the transmembrane protein. In an alternative embodiment of the invention disclosed herein the viral vector is derived from HIV: HIV differs from EIAV in that it does not encode S2 but unlike EIAV it encodes vif, vpr, vpu and nef.

The term “recombinant retroviral or lentiviral vector” (RRV) refers to a vector with sufficient retroviral genetic information to allow packaging of an RNA genome, in the presence of packaging components, into a viral particle capable of transducing a target cell. Transduction of the target cell may include reverse transcription and integration into the target cell genome. The RRV carries non-viral coding sequences which are to be delivered by the vector to the target cell. A RRV is incapable of independent replication to produce infectious retroviral particles within the target cell. Usually the RRV lacks a functional gag/pol and/or env gene, and/or other genes essential for replication.

Preferably the RRV vector of the invention disclosed herein has a minimal viral genome.

As used herein, the term “minimal viral genome” means that the viral vector has been manipulated so as to remove the non-essential elements whilst retaining the elements essential to provide the required functionality to infect, transduce and deliver a NOI to a target cell. Further details of this strategy can be found in WO 1998/17815 and WO 99/32646. A minimal EIAV vector lacks tat, rev and S2 genes and neither are these genes provided in trans in the production system. A minimal HIV vector lacks vif, vpr, vpu, tat and nef.

The expression plasmid used to produce the vector genome within a production cell may include transcriptional regulatory control sequences operably linked to the retroviral genome to direct transcription of the genome in a production cell/packaging cell. These regulatory sequences may be the natural sequences associated with the transcribed retroviral sequence, i.e. the 5′ U3 region, or they may be a heterologous promoter such as another viral promoter, for example the CMV promoter, as discussed below. Some lentiviral vector genomes require additional sequences for efficient virus production. For example, particularly in the case of HIV, RRE sequences may be included. However, the requirement for RRE (and dependence on rev which is provided in trans) may be reduced or eliminated by codon optimisation. Further details of this strategy can be found in WO 2001/79518.

Alternative sequences which perform the same function as the rev/RRE system are also known. For example, a functional analogue of the rev/RRE system is found in the Mason Pfizer monkey virus. This is known as the constitutive transport element (CTE) and comprises an RRE-type sequence in the genome which is believed to interact with a factor in the infected cell. The cellular factor can be thought of as a rev analogue. Thus, CTE may be used as an alternative to the rev/RRE system. Any other functional equivalents of the Rev protein which are known or become available may be relevant to the invention. For example, it is also known that the Rex protein of HTLV-1 can functionally replace the Rev protein of HIV-1. Rev and RRE may be absent or non-functional in the vector for use in the methods of the invention disclosed herein; in the alternative rev and RRE, or functionally equivalent systems, may be incorporated in vector systems herein.

As used herein, the term “functional substitute” means a protein or sequence having an alternative sequence which performs the same function as another protein or sequence. The term “functional substitute” is used interchangeably with “functional equivalent” and “functional analogue” herein with the same meaning.

### SIN Vectors

The retroviral vectors of the invention may be used in a self-inactivating (SIN) configuration in which the viral enhancer and promoter sequences have been deleted. SIN vectors can be generated and transduce non-dividing target cells in vivo, ex vivo or in vitro with an efficacy similar to that of non-SIN vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

By way of example, self-inactivating retroviral vector systems have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus. However, any promoter(s) internal to the LTRs in such vectors will still be transcriptionally active. This strategy has been employed to eliminate effects of the enhancers and promoters in the viral LTRs on transcription from internally placed genes. Such effects include increased transcription or suppression of transcription. This strategy can also be used to eliminate downstream transcription from the 3′ LTR into genomic DNA. This is of particular concern in human gene therapy where it is highly desirable, in some instances important, to prevent the adventitious activation of any endogenous oncogene. Yu et al., (1986) PNAS 83: 3194-98; Marty et al., (1990) Biochimie 72: 885-7; Naviaux et al., (1996) J. Virol. 70: 5701-5; Iwakuma et al., (1999) Virol. 261: 120-32; Deglon et al., (2000) Human Gene Therapy 11: 179-90. SIN lentiviral vectors are described in U.S. Pat. Nos. 6,924,123 and 7,056,699.

### Replication-Defective Lentiviral Vectors

In the genome of a replication-defective lentiviral vector the sequences of gag/pol and/or env may be mutated and/or not functional.

In a typical lentiviral vector of the invention disclosed herein, at least part of one or more coding regions for proteins essential for virus replication may be removed from the vector. This makes the viral vector replication-defective. Portions of the viral genome may also be replaced by a NOI in order to generate a vector comprising an NOI which is capable of transducing a non-dividing target cell and/or integrating its genome into the target cell genome.

In one embodiment the lentiviral vectors are non-integrating vectors as described in WO 2006/010834 and WO 2007/071994.

In a further embodiment the vectors have the ability to deliver a sequence which is devoid of or lacking viral RNA. In a further embodiment a heterologous binding domain (heterologous to gag) located on the RNA to be delivered and a cognate binding domain on Gag or GagPol can be used to ensure packaging of the RNA to be delivered. Both of these vectors are described in WO 2007/072056.

### Adenoviral and Adeno-Associated Viral Vectors

The use of recombinant adeno-associated viral (AAV) and Adenovirus based viral vectors for gene therapy is widespread, and manufacture of the same has been well documented. Typically, AAV-based vectors are produced in mammalian cell lines (e.g. HEK293-based) or through use of the baculovirus/Sf9 insect cell system. AAV vectors can be produced by transient transfection of vector component encoding DNAs, typically together with helper functions from Adenovirus or Herpes Simplex virus (HSV), or by use of cell lines stably expressing AAV vector components.

An AAV vector it is commonly understood to be a vector derived from an adeno-associated virus serotype, including without limitation, AAV-1, AAV-2, AAV-3, AAV-4, AAV-5, AAV-6, AAV-7 and AAV-8. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging. An ‘AAV vector’ also refers to its protein shell or capsid, which provides an efficient vehicle for delivery of vector nucleic acid to the nucleus of target cells. AAV production systems require helper functions which typically refers to AAV-derived coding sequences which can be expressed to provide AAV gene products that, in turn, function in trans for productive AAV replication. As such, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions are used herein to complement AAV functions in trans that are missing from AAV vectors. It is understood that a AAV helper construct refers generally to a nucleic acid molecule that includes nucleotide sequences providing AAV functions deleted from an AAV vector which is to be used to produce a transducing vector for delivery of a nucleotide sequence of interest. AAV helper constructs are commonly used to provide transient expression of AAV rep and/or cap genes to complement missing AAV functions that are necessary for AAV replication; however, helper constructs lack AAV ITRs and can neither replicate nor package themselves. AAV helper constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al. (1989) J. Virol. 63:3822-3828; and McCarty et al. (1991) J. Virol. 65:2936-2945, incorporated herein by reference. A number of other vectors have been described which encode Rep and/or Cap expression products. See, e.g., U.S. Pat. Nos. 5,139,941 and 6,376,237, incorporated herein by reference. In addition, it is common knowledge that the term “accessory functions” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

The following publications, incorporated herein by reference, describe various aspects of adeno-associated virus biology and/or techniques relating to the production of adeno-associated viral vectors. Aponte-Ubillus, et al. Molecular design for recombinant adeno-associated virus (rAAV) vector production. Appl Microbiol Biotechnol. 2018; 102(3): 1045-1054; Wang Q, et al. A Robust System for Production of Superabundant VP1 Recombinant AAV Vectors. Mol Ther Methods Clin Dev. 2017 Dec. 15; 7: 146-156; Adamson-Small L, et al. A scalable method for the production of high-titer and high-quality adeno-associated type 9 vectors using the HSV platform. Mol Ther Methods Clin Dev. 2016; 3: 16031; Clement N and Grieger J C. Manufacturing of recombinant adeno-associated viral vectors for clinical trials. Mol Ther Methods Clin Dev. 2016; 3: 16002; Guo P, et al. Rapid and simplified purification of recombinant adeno-associated virus. J Virol Methods. 2012 August; 183(2): 139-146; Shin J-H, et al. Recombinant Adeno-Associated Viral Vector Production and Purification. Methods Mol Biol. 2012; 798: 267-284; Cecchini S, et al. Reproducible High Yields of Recombinant Adeno-Associated Virus Produced Using Invertebrate Cells in 0.02- to 200-Liter Cultures. Hum. Gene Ther. 22(8) 2011: 1021-1030; Wright J F. Adeno-Associated Viral Vector Manufacturing: Keeping Pace with Accelerating Clinical Development. Hum. Gene Ther. 22(8) 2011: 913-915; Kotin R L. Large-scale recombinant adeno-associated virus production. Hum Mol Genet. 2011 Apr. 15; 20(R1): R2-R6; Thomas D L, et al. Scalable Recombinant Adeno-Associated Virus Production Using Recombinant Herpes Simplex Virus Type 1 Coinfection of Suspension-Adapted Mammalian Cells. Hum. Gene Ther. 20(8) 2009: 861-870; Thorne B A, et al. Manufacturing Recombinant Adeno-Associated Viral Vectors from Producer Cell Clones. Hum. Gene Ther. 20(7) 2009: 707-714; Wright F. Transient Transfection Methods for Clinical Adeno-Associated Viral Vector Production. Hum. Gene Ther. 20(7) 2009: 698-706; Urabe M, et al. Insect Cells As a Factory to Produce AdenoAssociated Virus Type 2 Vectors. Hum. Gene Ther. 13(16) 2002: 1935-1943.

Adenoviral vectors are typically produced in mammalian cell lines that stably express Adenovirus E1 functions (e.g. HEK293-based). Adenoviral vectors are also typically ‘amplified’ via helper-function-dependent replication through serial rounds of ‘infection’ using the production cell line. An adenoviral vector and production system thereof comprises a polynucleotide comprising all or a portion of an adenovirus genome. It is well known that an adenovirus is, without limitation, an adenovirus derived from Ad2, Ad5, Ad12, and Ad40. An adenoviral vector is typically in the form of DNA encapsulated in an adenovirus coat or adenoviral DNA packaged in another viral or viral-like form (such as herpes simplex, and AAV).

The following publications, incorporated herein by reference, describe various aspects of adenovirus biology and/or techniques relating to the production of adenoviral vectors. Graham and Van de Eb (1973) Virology 52:456-467; Takiff et al. (1981) Lancet ii: 832-834; Berkner and Sharp (1983) Nucleic Acid Research 6003-6020; Graham (1984) EMBO J 3:2917-2922; Bett et al. (1993) J. Virology 67:5911-5921; and Bett et al. (1994) Proc. Natl. Acad Sci. USA 91:8802-8806. Adenoviruses have been genetically modified to produce replication-defective gene transfer vectors. In such vectors, the early adenovirus gene products E1A and E1B are deleted and provided in trans by the packaging cell line 293 developed by Frank Graham (Graham et al. (1987) J. Gen. Birol. 36:59-72 and Graham (1977) J. Genetic Virology 68:937-940). The gene to be transduced is commonly inserted into the adenovirus in the deleted E1A and E1B region of the virus genome. Bett et al. (1994), supra. Adenoviral vectors and the production thereof have been described by Stratford-Perricaudet (1990) Human Gene Therapy 1:2-256; Rosenfeld (1991) Science 252:431-434; Wang et al. (1991) Adv. Exp. Med. Biol. 309:61-66; Jaffe et al. (1992) Nat Gent. 1:372-378; Quantin et al. (1992) Proc Natl. Acad. Sci. USA 89:2581-2584; Rosenfeld et al. (1992) Cell 68:143-155; Stratford-Perricaudet et al. (1992) J. Clin. Invest. 90:626-630; Le Gal La Salle et al. (1993) Science 259:988-990; Mastrangeli et al. (1993) J. Clin. Invest. 91:225-234; Ragot et al. (1993) Nature 361:647-650; Hayaski et al. (1994) J. Biol. Chem. 269:23872-23875; Lee C S, et al. Adenovirus-Mediated Gene Delivery: Potential Applications for Gene and Cell-Based Therapies in the New Era of Personalized Medicine. Genes Dis. 2017 June; 4(2):43-63; Kallel H, Kamen A A. Large-Scale Adenovirus and Poxvirus-Vectored Vaccine Manufacturing to Enable Clinical Trials. Biotechnol. J. 10(5) 2015: 741-747; Miravet S, et al. Construction, production, and purification of recombinant adenovirus vectors. Methods Mol Biol. 2014; 1089:159-73; Silva A C, et al. Scalable production of adenovirus vectors. Methods Mol Biol. 2014; 1089:175-96; Kreppel F. Production of high-capacity adenovirus vectors. Methods Mol Biol. 2014; 1089:211-29; Xie L, et al. Large-Scale Propagation of a Replication-Defective Adenovirus Vector in Stirred-Tank Bioreactor PER.C6™ Cell Culture Under Sparging Conditions. Biotechnol. Bioeng. 83(1) 2003: 45-52; Garnier A, et al. Scale-Up of the Adenovirus Expression System for the Production of Recombinant Protein in Human 293S Cells. Cell Culture Engineering IV. Buckland B C, Ed. Springer Science and Business Media: Rotterdam, The Netherlands, 1994; 145-155.

### NOI and Polynucleotides

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing PCR under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

### Proteins

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds.

### Variants, Derivatives, Analogues, Homologues and Fragments

In addition to the specific proteins and nucleotides mentioned herein, the invention disclosed herein also encompasses the use of variants, derivatives, analogues, homologues and fragments thereof.

In the context of the invention herein, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally-occurring protein.

The term “derivative” as used herein, in relation to proteins or polypeptides of the invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence retains the required activity or ability. Amino acid substitutions may include the use of non-naturally occurring analogues.

Proteins used in the invention disclosed herein may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar—uncharged | C S T M |
| | | N Q |
| | Polar—charged | D E |
| | | K R H |
| AROMATIC | | F W Y |

The term “homologue” means an entity having a certain homology with the wild type amino acid sequence and the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95%, 97 or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the invention disclosed herein it is preferred to express homology in terms of sequence identity.

In the present context, a homologous sequence is taken to include a nucleotide sequence which may be at least 50%, 55%, 65%, 75%, 85% or 90% identical, preferably at least 95%, 97% or 99% identical to the subject sequence. Although homology can also be considered in terms of similarity, in the context of the invention disclosed herein it is preferred to express homology in terms of sequence identity.

Homology comparisons can be conducted by eye, or more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Research 12:387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol Lett (1999) 174(2):247-50; FEMS Microbiol Lett (1999) 177(1):187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software usually does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the break. The DNA is then expressed in accordance with the invention to make the encoded protein. These methods are only illustrative of the numerous standard techniques known in the art for manipulation of DNA sequences and other known techniques may also be used.

All variants, fragments or homologues of the regulatory protein suitable for use in the cells and/or modular constructs of the invention will retain the ability to bind the cognate binding site of the NOI such that translation of the NOI is repressed or prevented in a viral vector production cell.

All variants fragments or homologues of the binding site will retain the ability to bind the cognate RNA-binding protein, such that translation of the NOI is repressed or prevented in a viral vector production cell.

### Codon Optimisation

The polynucleotides used in the invention disclosed herein (including the NOI and/or components of the vector production system) may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including retroviruses, use a large number of rare codons and changing these to correspond to commonly used mammalian codons, increases expression of a gene of interest, e.g. a NOI or packaging components in mammalian production cells, can be achieved. Codon usage tables are known in the art for mammalian cells, as well as for a variety of other organisms.

Codon optimisation of viral vector components has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. In lentiviral vectors codon optimisation also overcomes the Rev/RRE requirement for export, rendering optimised sequences Rev-independent. Codon optimisation also reduces homologous recombination between different constructs within the vector system (for example between the regions of overlap in the gag-pol and env open reading frames). The overall effect of codon optimisation is therefore a notable increase in viral titre and improved safety.

In one embodiment only codons relating to INS are codon optimised. However, in a much more preferred and practical embodiment, the sequences are codon optimised in their entirety, with some exceptions, for example the sequence encompassing the frameshift site of gag-pol (see below).

The gag-pol gene of lentiviral vectors comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimised. Retaining this fragment will enable more efficient expression of the Gag-Pol proteins. For EIAV the beginning of the overlap has been taken to be nt 1262 (where nucleotide 1 is the A of the gag ATG) and the end of the overlap to be nt 1461. In order to ensure that the frameshift site and the gag-pol overlap are preserved, the wild type sequence has been retained from nt 1156 to 1465.

Derivations from optimal codon usage may be made, for example, in order to accommodate convenient restriction sites, and conservative amino acid changes may be introduced into the Gag-Pol proteins.

In one embodiment, codon optimisation is based on lightly expressed mammalian genes. The third and sometimes the second and third base may be changed.

Due to the degenerate nature of the genetic code, it will be appreciated that numerous gag-pol sequences can be achieved by a skilled worker. Also there are many retroviral variants described which can be used as a starting point for generating a codon-optimised gag-pol sequence. Lentiviral genomes can be quite variable. For example there are many quasi-species of HIV-1 which are still functional. This is also the case for EIAV. These variants may be used to enhance particular parts of the transduction process. Examples of HIV-1 variants may be found at the HIV Databases operated by Los Alamos National Security, LLC at http://hiv-web.lanl.gov. Details of EIAV clones may be found at the National Center for Biotechnology Information (NCBI) database located at http://www.ncbi.nlm.nih.gov.

The strategy for codon-optimised gag-pol sequences can be used in relation to any retrovirus. This would apply to all lentiviruses, including EIAV, FIV, BIV, CAEV, VMV SIV, HIV-1 and HIV-2. In addition this method could be used to increase expression of genes from HTLV-1, HTLV-2, HFV, HSRV and human endogenous retroviruses (HERV), MLV and other retroviruses.

Codon optimisation can render gag-pol expression Rev-independent. In order to enable the use of anti-rev or RRE factors in the lentiviral vector, however, it would be necessary to render the viral vector generation system totally Rev/RRE-independent. Thus, the genome also needs to be modified. This is achieved by optimising vector genome components. Advantageously, these modifications also lead to the production of a safer system absent of all additional proteins both in the producer and in the transduced cell.

### Common Vector Elements

### Promoters and Enhancers

Expression of a NOI and polynucleotide may be controlled using control sequences for example transcription regulation elements or translation repression elements, which include promoters, enhancers and other expression regulation signals (e.g. tet repressor (TetR) system) or the Transgene Repression In vector Production cell system (TRIP) or other regulators of NOIs described herein.

Prokaryotic promoters and promoters functional in eukaryotic cells may be used. Tissue-specific or stimuli-specific promoters may be used. Chimeric promoters may also be used comprising sequence elements from two or more different promoters.

Suitable promoting sequences are strong promoters including those derived from the genomes of viruses, such as polyoma virus, adenovirus, fowlpox virus, bovine papilloma virus, avian sarcoma virus, cytomegalovirus (CMV), retrovirus and Simian Virus 40 (SV40), or from heterologous mammalian promoters, such as the actin promoter, EF1α, CAG, TK, SV40, ubiquitin, PGK or ribosomal protein promoter. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VMD2), Tyrosine hydroxylase, neuronal-specific neuronal-specific enolase (NSE) promoter, astrocyte-specific glial fibrillary acidic protein (GFAP) promoter, human al-antitrypsin (hAAT) promoter, phosphoenolpyruvate carboxykinase (PEPCK), liver fatty acid binding protein promoter, Flt-1 promoter, INF-β promoter, Mb promoter, SP-B promoter, SYN1 promoter, WASP promoter, SV40/hAlb promoter, SV40/CD43, SV40/CD45, NSE/RU5′ promoter, ICAM-2 promoter, GPIIb promoter, GFAP promoter, Fibronectin promoter, Endoglin promoter, Elastase-1 promoter, Desmin promoter, CD68 promoter, CD14 promoter and B29 promoter may be used to drive transcription.

Transcription of a NOI may be increased further by inserting an enhancer sequence into the vector. Enhancers are relatively orientation- and position-independent; however, one may employ an enhancer from a eukaryotic cell virus, such as the SV40 enhancer and the CMV early promoter enhancer. The enhancer may be spliced into the vector at a position 5′ or 3′ to the promoter, but is preferably located at a site 5′ from the promoter.

The promoter can additionally include features to ensure or to increase expression in a suitable target cell. For example, the features can be conserved regions e.g. a Pribnow Box or a TATA box. The promoter may contain other sequences to affect (such as to maintain, enhance or decrease) the levels of expression of a nucleotide sequence. Suitable other sequences include the Sh1-intron or an ADH intron. Other sequences include inducible elements, such as temperature, chemical, light or stress inducible elements. Also, suitable elements to enhance transcription or translation may be incorporated herein.

### Regulators of NO/s

A complicating factor in the generation of retroviral packaging/producer cell lines and retroviral vector production is that constitutive expression of certain retroviral vector components and NOIs are cytotoxic leading to death of cells expressing these components and therefore inability to produce vector. Therefore, for some embodiments of the present invention, the expression of these components (e.g. gag-pol and envelope proteins such as VSV-G) has to be regulated. The expression of other non-cytotoxic vector components can also be regulated to minimise the metabolic burden on the cell. The modular constructs and/or cells of the invention may comprise cytotoxic and/or non-cytotoxic vector components associated with at least one regulatory element. As used herein, the term “regulatory element” refers to any element capable of affecting, either increasing or decreasing, the expression of an associated gene or protein. A regulatory element includes a gene switch system, transcription regulation element and translation repression element

A number of prokaryotic regulator systems have been adapted to generate gene switches in mammalian cells. Many retroviral packaging and producer cell lines have been controlled using gene switch systems (e.g. tetracycline and cumate inducible switch systems) thus enabling expression of one or more of the retroviral vector components to be switched on at the time of vector production. Gene switch systems include those of the (TetR) protein group of transcription regulators (e.g. T-Rex, Tet-On, and Tet-Off) and those of the cumate inducible switch system group of transcription regulators (e.g. CymR protein).

One such tetracycline-inducible system is the tetracycline repressor (TetR) system based on the T-REx™ system. By way of example, in such a system tetracycline operators (TetO₂) are placed in a position such that the first nucleotide is 10 bp from the 3′ end of the last nucleotide of the TATATAA element of the human cytomegalovirus major immediate early promoter (hCMVp) then TetR alone is capable of acting as a repressor (Yao F, Svensjo T, Winkler T, Lu M, Eriksson C, Eriksson E. Tetracycline repressor, tetR, rather than the tetR-mammalian cell transcription factor fusion derivatives, regulates inducible gene expression in mammalian cells. 1998. Hum Gene Ther, 9: 1939-1950). In such a system the expression of the NOI can be controlled by a CMV promoter into which two copies of the TetO₂ sequence have been inserted in tandem. TetR homodimers, in the absence of an inducing agent (tetracycline or its analogue doxycycline [dox]), bind to the TetO₂ sequences and physically block transcription from the upstream CMV promoter. When present, the inducing agent binds to the TetR homodimers, causing allosteric changes such that it can no longer bind to the TetO₂ sequences, resulting in gene expression. The TetR gene used in the Examples disclosed herein has been codon optimised as this was found to improve translation efficiency resulting in tighter control of TetO₂ controlled gene expression.

The TRiP system is described in WO 2015/092440 and provides another way of repressing expression of the NOI in the production cells during vector production. The TRAP-binding sequence (e.g. TRAP-tbs) interaction forms the basis for a transgene protein repression system for the production of retroviral vectors, when a constitutive and/or strong promoter, including a tissue-specific promoter, driving the transgene is desirable and particularly when expression of the transgene protein in production cells leads to reduction in vector titres and/or elicits an immune response in vivo due to viral vector delivery of transgene-derived protein (Maunder et al, Nat Commun. (2017) March 27; 8).

Briefly, the TRAP-tbs interaction forms a translational block, repressing translation of the transgene protein (Maunder et al, Nat Commun. (2017) March 27; 8). The translational block is only effective in production cells and as such does not impede the DNA- or RNA-based vector systems. The TRiP system is able to repress translation when the transgene protein is expressed from a constitutive and/or strong promoter, including a tissue-specific promoter from single- or multi cistronic mRNA. It has been demonstrated that unregulated expression of transgene protein can reduce vector titres and affect vector product quality. Repression of transgene protein for both transient and stable PaCL/PCL vector production systems is beneficial for production cells to prevent a reduction in vector titres: where toxicity or molecular burden issues may lead to cellular stress; where transgene protein elicits an immune response in vivo due to viral vector delivery of transgene-derived protein; where the use of gene-editing transgenes may result in on/off target affects; where the transgene protein may affect vector and/or envelope glycoprotein exclusion.

### Envelope and Pseudotyping

In one preferred aspect, the retroviral vector of the invention disclosed herein has been pseudotyped. In this regard, pseudotyping can confer one or more advantages. For example, the env gene product of the HIV based vectors would restrict these vectors to infecting only cells that express a protein called CD4. But if the env gene in these vectors has been substituted with env sequences from other enveloped viruses, then they may have a broader infectious spectrum (Verma and Somia (1997) Nature 389(6648):239-242). By way of example, workers have pseudotyped an HIV based vector with the glycoprotein from VSV (Verma and Somia (1997) Nature 389(6648):239-242).

In another alternative, the Env protein may be a modified Env protein such as a mutant or engineered Env protein. Modifications may be made or selected to introduce targeting ability or to reduce toxicity or for another purpose (Valsesia-Wittman et al 1996 J Virol 70: 2056-64; Nilson et al (1996) Gene Ther 3(4):280-286; and Fielding et al (1998) Blood 91(5):1802-1809 and references cited therein).

The vector may be pseudotyped with any molecule of choice.

As used herein, “env” shall mean an endogenous lentiviral envelope or a heterologous envelope, as described herein.

### VSV-G

The envelope glycoprotein (G) of Vesicular stomatitis virus (VSV), a rhabdovirus, is an envelope protein that has been shown to be capable of pseudotyping certain enveloped viruses and viral vector virions.

Its ability to pseudotype MoMLV-based retroviral vectors in the absence of any retroviral envelope proteins was first shown by Emi et al. (1991) Journal of Virology 65:1202-1207). WO 1994/294440 teaches that retroviral vectors may be successfully pseudotyped with VSV-G. These pseudotyped VSV-G vectors may be used to transduce a wide range of mammalian cells. More recently, Abe et al. (1998) J Virol 72(8) 6356-6361 teach that non-infectious retroviral particles can be made infectious by the addition of VSV-G.

Burns et al. (1993) Proc. Natl. Acad. Sci. USA 90:8033-7) successfully pseudotyped the retrovirus MLV with VSV-G and this resulted in a vector having an altered host range compared to MLV in its native form. VSV-G pseudotyped vectors have been shown to infect not only mammalian cells, but also cell lines derived from fish, reptiles and insects (Burns et al. (1993) ibid). They have also been shown to be more efficient than traditional amphotropic envelopes for a variety of cell lines (Yee et al., (1994) Proc. Natl. Acad. Sci. USA 91:9564-9568, Emi et al. (1991) Journal of Virology 65:1202-1207). VSV-G protein can be used to pseudotype certain retroviruses because its cytoplasmic tail is capable of interacting with the retroviral cores.

The provision of a non-retroviral pseudotyping envelope such as VSV-G protein gives the advantage that vector particles can be concentrated to a high titre without loss of infectivity (Akkina et al. (1996) J. Virol. 70:2581-5). Retrovirus envelope proteins are apparently unable to withstand the shearing forces during ultracentrifugation, probably because they consist of two non-covalently linked subunits. The interaction between the subunits may be disrupted by the centrifugation. In comparison the VSV glycoprotein is composed of a single unit. VSV-G protein pseudotyping can therefore offer potential advantages for both efficient target cell infection/transduction and during manufacturing processes.

WO 2000/52188 describes the generation of pseudotyped retroviral vectors, from stable producer cell lines, having vesicular stomatitis virus-G protein (VSV-G) as the membrane-associated viral envelope protein, and provides a gene sequence for the VSV-G protein.

### Ross River Virus

The Ross River viral envelope has been used to pseudotype a non-primate lentiviral vector (FIV) and following systemic administration predominantly transduced the liver (Kang et al (2002) J Virol 76(18):9378-9388). Efficiency was reported to be 20-fold greater than obtained with VSV-G pseudotyped vector, and caused less cytotoxicity as measured by serum levels of liver enzymes suggestive of hepatotoxicity.

### Baculovirus GP64

The baculovirus GP64 protein has been shown to be an alternative to VSV-G for viral vectors used in the large-scale production of high-titre virus required for clinical and commercial applications (Kumar M, Bradow B P, Zimmerberg J (2003) Hum Gene Ther. 14(1):67-77). Compared with VSV-G-pseudotyped vectors, GP64-pseudotyped vectors have a similar broad tropism and similar native titres. Because, GP64 expression does not kill cells, HEK293T-based cell lines constitutively expressing GP64 can be generated.

### Alternative Envelopes

Other envelopes which give reasonable titre when used to pseudotype EIAV include Mokola, Rabies, Ebola and LCMV (lymphocytic choriomeningitis virus). Intravenous infusion into mice of lentivirus pseudotyped with 4070A led to maximal gene expression in the liver.

### Packaging Sequence

As utilized within the context of the invention the term “packaging signal”, which is referred to interchangeably as “packaging sequence” or “psi”, is used in reference to the non-coding, cis-acting sequence required for encapsidation of retroviral RNA strands during viral particle formation. In HIV-1, this sequence has been mapped to loci extending from upstream of the major splice donor site (SD) to at least the gag start codon. In EIAV the packaging signal comprises the R region into the 5′ coding region of Gag.

As used herein, the term “extended packaging signal” or “extended packaging sequence” refers to the use of sequences around the psi sequence with further extension into the gag gene. The inclusion of these additional packaging sequences may increase the efficiency of insertion of vector RNA into viral particles.

Feline immunodeficiency virus (FIV) RNA encapsidation determinants have been shown to be discrete and non-continuous, comprising one region at the 5′ end of the genomic mRNA (R-U5) and another region that mapped within the proximal 311 nt of gag (Kaye et al., J Virol. October; 69(10):6588-92 (1995).

### Internal Ribosome Entry Site (IRES)

Insertion of IRES elements allows expression of multiple coding regions from a single promoter (Adam et al (as above); Koo et al (1992) Virology 186:669-675; Chen et al 1993 J. Virol 67:2142-2148). IRES elements were first found in the non-translated 5′ ends of picornaviruses where they promote cap-independent translation of viral proteins (Jang et al (1990) Enzyme 44: 292-309). When located between open reading frames in an RNA, IRES elements allow efficient translation of the downstream open reading frame by promoting entry of the ribosome at the IRES element followed by downstream initiation of translation.

A review on IRES is presented by Mountford and Smith (TIG May 1995 vol 11, No 5:179-184). A number of different IRES sequences are known including those from encephalomyocarditis virus (EMCV) (Ghattas, I. R., et al., Mol. Cell. Biol., 11:5848-5859 (1991); BiP protein [Macejak and Sarnow, Nature 353:91 (1991)]; the Antennapedia gene of Drosophila (exons d and e) [Oh, et al., Genes & Development, 6:1643-1653 (1992)] as well as those in polio virus (PV) [Pelletier and Sonenberg, Nature 334: 320-325 (1988); see also Mountford and Smith, TIG 11, 179-184 (1985)].

IRES elements from PV, EMCV and swine vesicular disease virus have previously been used in retroviral vectors (Coffin et al, as above).

The term “IRES” includes any sequence or combination of sequences which work as or improve the function of an IRES. The IRES(s) may be of viral origin (such as EMCV IRES, PV IRES, or FMDV 2A-like sequences) or cellular origin (such as FGF2 IRES, NRF IRES, Notch 2 IRES or EIF4IRES).

In order for the IRES to be capable of initiating translation of each polynucleotide it should be located between or prior to the polynucleotides in the modular construct.

Vector constructs disclosed utilised for development of stable cell lines require the addition of selectable markers for selection of cells where stable integration has occurred. These selectable markers can be expressed as a single transcription unit within the modular construct or it may be preferable to use IRES elements to initiate translation of the selectable marker in a polycistronic message (Adam et al 1991 J. Virol. 65, 4985). As such, a number of the modular constructs were designed such that the selectable maker was placed downstream of one of the retroviral vector components utilising an IRES element.

### Genetic Orientation and Insulators

It is well known that nucleic acids are directional and this ultimately affects mechanisms such as transcription and replication in the cell. Thus genes can have relative orientations with respect to one another when part of the same nucleic acid construct.

As such, at least two nucleic acid sequences present at the same locus in the cell or construct can be in a reverse and/or alternating orientations. In other words, at this particular locus, the pair of sequential genes will not have the same orientation. Alternating orientations benefits retroviral vector production when the nucleic acids required for vector production are based at the same genetic locus within the production cell. This can help prevent both transcriptional and translational read-through when the region is expressed within the same physical location of the production cell. This in turn can also improve the safety of the resulting constructs in preventing the generation of replication-competent retroviral vectors. When nucleic acid sequences are in reverse and/or alternating orientations the use of insulators can prevent inappropriate expression or silencing of a NOI from its genetic surroundings.

The term “insulator” refers to a class of DNA sequence elements that when bound to insulator-binding proteins possess an ability to protect genes from surrounding regulator signals. There are two types of insulators: an enhancer blocking function and a chromatin barrier function. When an insulator is situated between a promoter and an enhancer, the enhancer-blocking function of the insulator shields the promoter from the transcription-enhancing influence of the enhancer (Geyer and Corces 1992; Kellum and Schedl 1992). The chromatin barrier insulators function by preventing the advance of nearby condensed chromatin which would lead to a transcriptionally active chromatin region turning into a transcriptionally inactive chromatin region and resulting in silencing of gene expression. Insulators which inhibit the spread of heterochromatin, and thus gene silencing, recruit enzymes involved in histone modifications to prevent this process (Yang J, Corces V G. Chromatin Insulators: A Role in Nuclear Organization and Gene Expression. Advances in cancer research. 2011; 110:43-76. doi:10.1016/B978-0-12-386469-7.00003-7; Huang, Li et al. 2007; Dhillon, Raab et al. 2009). An insulator can have one or both of these functions and the chicken β-globin insulator (cHS4) is one such example. This insulator is the most extensively studied vertebrate insulator, is highly rich in G+C and has both enhancer-blocking and heterochromatic barrier functions (Chung J H, Whitely M, Felsenfeld G. Cell. 1993; 74:505-514). Other such insulators with enhancer blocking functions are not limited to but include the following: human β-globin insulator 5 (HS5), human β-globin insulator 1 (HS1), and chicken β-globin insulator (cHS3) (Farrell CM1, West A G, Felsenfeld G., Mol Cell Biol. 2002 June; 22(11):3820-31; J Ellis et al. EMBO J. 1996 Feb. 1; 15(3): 562-568). In addition to reducing unwanted distal interactions the insulators also help to prevent promoter interference (i.e. where the promoter from one transcription unit impairs expression of an adjacent transcription unit) between adjacent retroviral nucleic acid sequences. If the insulators are used between each of the retroviral vector nucleic acid sequences, then the reduction of direct read-through will help prevent the formation of replication-competent retroviral vector particles. An insulator can be present between each of the retroviral nucleic acid sequences and the use of insulators may prevent promoter-enhancer interactions from one NOI expression cassette interacting with another NOI expression cassette in a modular construct. An insulator may be present between the vector genome and gag-pol sequences. This therefore limits the likelihood of the production of a replication-competent retroviral vector and ‘wild-type’ like RNA transcripts, improving the safety profile of the construct. The use of insulator elements to improve the expression of stably integrated multigene vectors is cited in Moriarity et al (Modular assembly of transposon integratable multigene vectors using RecWay assembly, Nucleic Acids Res. 2013 April; 41(8):e92).

### Vector Titre

The skilled person will understand that there are a number of different methods of determining the titre of viral vectors. Titre is often described as transducing units/mL (TU/mL). Titre may be increased by increasing the number of vector particles and by increasing the specific activity of a vector preparation.

### Modular Constructs

The nuclease-secreting viral production system and methods disclosed herein may incorporate modular nucleic acid constructs (modular constructs) disclosed in co-pending EP 17210359.0 application number, entitled RETROVIRAL VECTOR, incorporated by reference in its entirety herein. A modular construct is a DNA expression construct comprising two or more nucleic acids used in the production of retroviral vectors. A modular construct can be a DNA plasmid comprising two or more nucleic acids used in the production of retroviral vectors. The nucleic acids can encode for example, gag-pol, rev, env, vector genome. In addition, modular constructs designed for generation of packaging and producer cell lines may additionally need to encode transcriptional regulatory proteins (e.g. TetR, CymR) and/or translational repression proteins (e.g. TRAP) and selectable markers (e.g Zeocin®, hygromycin, blasticidin, puromycin, neomycin resistance genes).

The DNA expression construct can be a DNA plasmid (supercoiled, nicked or linearised), minicircle DNA (linear or supercoiled), plasmid DNA containing just the regions of interest by removal of the plasmid backbone by restriction enzyme digestion and purification, DNA generated using an enzymatic DNA amplification platform e.g. doggybone DNA (dbDNA™) where the final DNA used is in a closed ligated form or where it has been prepared (e.g restriction enzyme digestion) to have open cut ends.

As described herein, current methods for retroviral vector production utilise genetic constructs in which genes essential for retroviral production are introduced into a production cell on separate plasmids by transient transfection methods. This can create batch-to-batch variation and further increases the cost due to the expensive transfection agents and plasmids. By using such modular constructs the number of plasmids which are needed in the transfection process are reduced, thus reducing the burden on labour and material cost.

The use of such modular constructs can also aid in the production of efficient packaging and producer cell lines. In particular, introducing two or more retroviral vector genes onto one modular construct will subsequently reduce the number of stable transfections/transductions, integrations, and selection steps required in order to create the final packaging/producer cell.

In particular, it has been surprising to find that bacterial plasmids are able to perform this function, as it is generally believed that the large genes involved would not permit multiple genes to be stably incorporated into a bacterial plasmid.

In accordance with an aspect of the invention, stable cell lines (packaging or producer) for producing the retroviral vectors comprise at least two of the retroviral genes located at the same genetic locus. The table below lists example combinations of nucleic acids which can be located at the same locus in stable vector production cells of the invention disclosed herein and which are expressed from a single modular construct. The order of each component nucleic acid is also as stated. The asterisk (*) marks combinations which would be suitable for the production of EIAV-based retroviral vectors, as Rev is not an essential component for such vectors. The double asterisk (**) marks combinations which are associated with a regulatory element or are in reverse and/or alternating orientations in the vector.

| Number of | |
|---|---|
| expression | |
| cassettes | Combination |
| 2 | Genome Rev |
| | Genome VSV-G* |
| | Genome Gag-Pol* |
| | Rev Genome |
| | Rev VSVG |
| | Rev Gag-Pol** |
| | VSVG Genome* |
| | VSVG Rev |
| | VSVG Gag-Pol* |
| | Gag-Pol Genome* |
| | Gag-Pol Rev** |
| | Gag-Pol VSVG* |
| 3 | Genome Rev VSVG |
| | Genome Rev Gag-Pol |
| | Genome VSVG Rev |
| | Genome VSVG Gag-Pol* |
| | Genome Gag-Pol Rev |
| | Genome Gag-Pol VSVG* |
| | Rev Genome VSVG |
| | Rev Genome Gag-Pol |
| | Rev VSVG Genome |
| | Rev VSVG Gag-Pol |
| | Rev Gag-Pol Genome |
| | Rev Gag-Pol VSVG |
| | VSVG Genome Rev |
| | VSVG Genome Gag-Pol* |
| | VSVG Rev Genome |
| | VSVG Rev Gag-Pol |
| | VSVG Gag-Pol Genome* |
| | VSVG Gag-Pol Rev |
| | Gag-Pol Genome Rev |
| | Gag-Pol Genome VSVG* |
| | Gag-Pol Rev Genome |
| | Gag-Pol Rev VSVG |
| | Gag-Pol VSVG Genome* |
| | Gag-Pol VSVG Rev |
| 4 | Genome Rev VSVG Gag-Pol |
| | Genome Rev Gag-Pol VSVG |
| | Genome VSVG Rev Gag-Pol |
| | Genome VSVG Gag-Pol Rev |
| | Genome Gag-Pol Rev VSVG |
| | Genome Gag-Pol VSVG Rev |
| | Rev Genome VSVG Gag-Pol |
| | Rev Genome Gag-Pol VSVG |
| | Rev VSVG Genome Gag-Pol |
| | Rev VSVG Gag-Pol Genome |
| | Rev Gag-Pol Genome VSVG |
| | Rev Gag-Pol VSVG Genome |
| | VSVG Genome Rev Gag-Pol |
| | VSVG Genome Gag-Pol Rev |
| | VSVG Rev Genome Gag-Pol |
| | VSVG Rev Gag-Pol Genome |
| | VSVG Gag-Pol Genome Rev |
| | VSVG Gag-Pol Rev Genome |
| | Gag-Pol Genome Rev VSVG |
| | Gag-Pol Genome VSVG Rev |
| | Gag-Pol Rev Genome VSVG |
| | Gag-Pol Rev VSVG Genome |
| | Gag-Pol VSVG Genome Rev |
| | Gag-Pol VSVG Rev Genome |

When using such modular constructs, the cell or vector of the invention does not contain an origin of replication sequence derived from a PAC, BAC, YAC, cosmid or fosmid. PAC, BAC, YAC, cosmid and fosmids are artificially generated nucleic acid vectors designed to hold large quantities of DNA. Thus, their core sequences are well known and defined in the art.

### Viral Vector Production Systems and Cells

Generally speaking, a “viral vector production system” or “vector production system” or “production system” is to be understood as a system comprising the necessary components for viral vector production.

In one embodiment of the invention, the viral vector production system is a retroviral vector production system which comprises nucleic acid sequences encoding Gag and Gag/Pol proteins, and Env protein thereof and the vector genome sequence. The production system may optionally comprise a nucleic acid sequence encoding the Rev protein, or functional substitute thereof.

In another embodiment, the retroviral vector is derived from a lentivirus. In another embodiment, the retroviral vector is derived from HIV-1, HIV-2, SIV, FIV, BIV, EIAV, CAEV or Visna lentivirus. Another aspect of the invention relates to a set of DNA constructs for use in the retroviral vector production system of the invention comprising the modular constructs of the invention. In one embodiment of the invention, the set of DNA constructs additionally comprises a DNA construct encoding Rev protein or a functional substitute thereof.

Another aspect of the invention relates to a retroviral vector production cell comprising the nucleic acid sequence, such as expression cassettes and/or some or all modular constructs, encoding the viral vector components.

In another embodiment of the invention, viral vector production system is an AAV viral vector production system or an adenoviral vector production system.

A “viral vector production cell”, “vector production cell”, or “production cell” is to be understood as a cell that is capable of producing a viral vector or viral vector particle. Retroviral vector production cells may be “producer cells” or “packaging cells”. One or more DNA constructs of the viral vector system may be either stably integrated or episomally maintained within the viral vector production cell. Alternatively, all the DNA components of the viral vector system may be transiently transfected into the viral vector production cell. In yet another alternative, a production cell stably expressing some of the components may be transiently transfected with the remaining components required for vector production.

As used herein, the term “packaging cell” refers to a cell which contains the elements necessary for production of retroviral vector particles but which lacks the vector genome. Optionally, such packaging cells contain one or more expression cassettes which are capable of expressing viral structural proteins (such as gag, gag/pol and env).

Producer cells/packaging cells can be of any suitable cell type. Producer cells are generally mammalian cells but can be, for example, insect cells.

As used herein, the term “producer/production cell” or “vector producing/production cell” refers to a cell which contains all the elements necessary for production of retroviral vector particles. The producer cell may be either a stable producer cell line or derived transiently or may be a stable packaging cell wherein the retroviral genome is transiently expressed.

The vector production cells may be cells cultured in vitro such as a tissue culture cell line. Suitable cell lines include, but are not limited to, mammalian cells such as murine fibroblast derived cell lines or human cell lines. Preferably the vector production cells are derived from a human cell line.

### Cells and Production Methods

The invention relates to a process for producing viral vectors comprising introducing the nucleic acid sequences described herein into a cell (e.g. a production cell) and culturing the cell under conditions suitable for the production of the viral vectors.

In a further aspect, the invention disclosed herein provides a replication defective retroviral vector produced by any method of the invention.

Suitable production cells are those cells which are capable of producing viral vectors or viral vector particles when cultured under appropriate conditions. They are generally eukaryotic cells such as mammalian or human cells, for example HEK293T, HEK293, CAP, CAP-T, CHO cells, or PER.C6 cells but can be, for example, insect cells such as SF9 cells.

Methods for introducing nucleic acids into production cells are well known in the art and have been described previously.

Stable cells may be packaging or producer cells. To generate producer cells from packaging cells the vector genome DNA construct may be introduced stably or transiently.

Packaging/producer cells can be generated by transducing a suitable cell line with a retroviral vector which expresses one of the components of the packaging/producer cell, i.e. a genome, the gag-pol components and an envelope as described in WO 2004/022761.

Alternatively, the nucleic acid can be transfected into cells and then integration into the production cell genome occurs infrequently and randomly. The transfection methods may be performed using methods well known in the art. For example, the transfection process may be performed using calcium phosphate or commercially available formulations such as Lipofectamine™ 200000 (Invitrogen, CA), FuGENE® HD or polyethylenimine (PEI). Alternatively modular constructs of the invention may be introduced into the production cell via electroporation. The skilled person will be aware of methods to encourage integration of the nucleic acids into production cells. For example, linearising a nucleic acid construct can help if it is naturally circular. Less random integration methodologies may involve the nucleic acid construct comprising of areas of shared homology with the endogenous chromosomes of the mammalian host cell to guide integration to a selected site within the endogenous genome. Furthermore, if recombination sites are present on the construct then these can be used for targeted recombination. For example, the nucleic acid construct may contain a loxP site which allows for targeted integration when combined with Cre recombinase (i.e. using the Cre/lox system derived from P1 bacteriophage). Alternatively or additionally, the recombination site is an att site (e.g. from A phage), wherein the att site permits site-directed integration in the presence of a lambda integrase. This would allow the retroviral genes to be targeted to a locus within the host cellular genome which allows for high and/or stable expression.

Other methods of targeted integration are well known in the art. For example, methods of inducing targeted cleavage of genomic DNA can be used to encourage targeted recombination at a selected chromosomal locus. These methods often involve the use of methods or systems to induce a double strand break (DSB) e.g. a nick in the endogenous genome to induce repair of the break by physiological mechanisms such as non-homologous end joining (NHEJ). Cleavage can occur through the use of site-specific nucleases such as engineered zinc finger nucleases (ZFN), transcription-activator like effector nucleases (TALENs), using CRISPR/Cas9 systems with an engineered crRNA/tracr RNA (‘single guide RNA’) to guide specific cleavage, and/or using nucleases based on the Argonaute system (e.g., from T. thermophilus). Such gene-editing-type nucleases are not suitable for use in the vector production cells for the purposes of non-specifically degrading residual nucleic acid during vector production because they are site-specific enzymes targeted to the nuclease and not secreted as are the non-specific nucleases of the invention disclosed herein.

Packaging/producer cell lines can be generated by integration of nucleic acids using methods of retroviral transduction or nucleic acid transfection, or a combination thereof. Methods for generating retroviral vectors from production cells and in particular the processing of retroviral vectors are described in WO 2009/153563.

A production cell may optionally comprise the RNA-binding protein (e.g. tryptophan RNA-binding attenuation protein, TRAP) and/or the Tet Repressor (TetR) protein or alternative regulatory proteins (e.g. CymR).

Production of retroviral vector from production cells can be via transfection methods, from production from stable cell lines which can include induction steps (e.g. doxycycline induction) or via a combination of both. The transfection methods may be performed using methods well known in the art, and examples have been described previously.

Production cells, either packaging or producer cell lines or those transiently transfected with the retroviral vector encoding components are cultured to increase cell and virus numbers and/or virus titres. Culturing a cell is performed to enable it to metabolize, and/or grow and/or divide and/or produce viral vectors of interest according to the invention. This can be accomplished by methods well known to persons skilled in the art, and includes but is not limited to providing nutrients for the cell, for instance in the appropriate culture media. The methods may comprise growth adhering to surfaces, growth in suspension, or combinations thereof. Culturing can be done for instance in tissue culture flasks, tissue culture multi-well plates, dishes, roller bottles, wave bags or in bioreactors, using batch, fed-batch, continuous systems and the like. In order to achieve large scale production of viral vector through cell culture it is preferred in the art to have cells capable of growing in suspension. Suitable conditions for culturing cells are known (see e.g. Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9).

In one embodiment, cells are initially ‘bulked up’ in tissue culture flasks or bioreactors and subsequently grown in multi-layered culture vessels or large bioreactors (greater than 50 L) to generate the vector producing cells of the invention disclosed herein.

In another embodiment, cells are grown in an adherent mode to generate the vector producing cells of the invention.

In yet another embodiment, cells are grown in a suspension mode to generate the vector producing cells of the invention.

### Use

Another aspect of the invention relates to the use of the viral vector of the invention or a cell or tissue transduced with the viral vector of the invention in medicine.

Another aspect of the invention relates to the use of the viral vector of the invention, a production cell of the invention or a cell or tissue transduced with the viral vector of the invention for the preparation of a medicament to deliver a nucleotide of interest to a target site in need of the same. Such uses of the viral vector or transduced cell of the invention may be for therapeutic or diagnostic purposes, as described previously.

Another aspect of the invention relates to a cell transduced by the viral vector of the invention.

A “cell transduced by a viral vector particle” is to be understood as a cell, in particular a target cell, into which the nucleic acid carried by the viral vector particle has been transferred.

### Pharmaceutical Compositions

Another aspect of the invention relates to a pharmaceutical composition comprising the viral vector of the invention or a cell or tissue transduced with the viral vector of the invention, in combination with a pharmaceutically acceptable carrier, diluent or excipient.

The invention disclosed herein also provides a pharmaceutical composition for treating an individual by gene therapy, wherein the composition comprises a therapeutically effective amount of a viral vector. The pharmaceutical composition may be for human or animal usage.

The composition may comprise a pharmaceutically acceptable carrier, diluent, excipient or adjuvant. The choice of pharmaceutical carrier, excipient or diluent can be made with regard to the intended route of administration and standard pharmaceutical practice. The pharmaceutical compositions may comprise, or be in addition to, the carrier, excipient or diluent any suitable binder(s), lubricant(s), suspending agent(s), coating agent(s), solubilising agent(s) and other carrier agents that may aid or increase vector entry into the target site (such as for example a lipid delivery system).

Where appropriate, the composition can be administered by any one or more of inhalation; in the form of a suppository or pessary; topically in the form of a lotion, solution, cream, ointment or dusting powder; by use of a skin patch; orally in the form of tablets containing excipients such as starch or lactose, or in capsules or ovules either alone or in admixture with excipients, or in the form of elixirs, solutions or suspensions containing flavouring or colouring agents; or they can be injected parenterally, for example intracavernosally, intravenously, intramuscularly, intracranially, intraoccularly intraperitoneally, or subcutaneously. For parenteral administration, the compositions may be best used in the form of a sterile aqueous solution which may contain other substances, for example enough salts or monosaccharides to make the solution isotonic with blood. For buccal or sublingual administration, the compositions may be administered in the form of tablets or lozenges which can be formulated in a conventional manner.

The viral vector of the invention may also be used to transduce target cells or target tissue ex vivo prior to transfer of said target cell or tissue into a patient in need of the same. An example of such target cells may be autologous T cells and an example of such tissue may be a donor cornea.

The present invention is useful in the production of gesicles or exosomes. In this respect, gesicles are typically made by over-expressing the VSV-G envelope in (HEK293-dereived) cell lines, which produces vesicles. Exosomes can be induced/expressed endogenously from cells. Both gesicles or exosomes can be loaded with RNAs and proteins, such as CRISPR-cas9 or other gene editing components.

### Numbered Paragraphs

Disclosed herein are viral vector production systems secreting nuclease for degradation of residual nucleic acid during viral vector production and methods of the same. Such a viral vector production system comprises a viral vector production cell comprising nucleic acid sequences encoding: 1) viral vector components; and 2) a nuclease, wherein the nuclease is expressed in the production cell and secreted in cell culture thereby degrading residual nucleic acid during viral vector production. Another such viral vector production system comprises 1) a viral vector production cell comprising nucleic acid sequences encoding viral vector components; and 2) a nuclease helper cell comprising a nucleic acid sequence encoding a nuclease, wherein the nuclease is expressed and secreted in co-culture of the production cell of 1) and the helper cell of 2), thereby degrading residual nucleic acid during viral vector production.

Aspects of the present invention will now be described by way of numbered paragraphs 1-26—presented as numbered paragraphs set 1.

### Numbered Paragraphs Set 1:

1. A viral vector production system comprising a viral vector production cell comprising nucleic acid sequences encoding: 1) viral vector components; and 2) a nuclease, wherein the nuclease is expressed in the production cell and secreted in cell culture thereby degrading residual nucleic acid during viral vector production.

2. A viral vector production system comprising: 1) a viral vector production cell comprising nucleic acid sequences encoding viral vector components; and 2) a nuclease helper cell comprising a nucleic acid sequence encoding a nuclease, wherein the nuclease is expressed and secreted in co-culture of the production cell of 1) and the helper cell of 2), thereby degrading residual nucleic acid during viral vector production.

3. A method of producing a viral vector, the method comprising, transfecting (sometimes referred to as contacting) a viral vector production cell with nucleic acid sequences encoding: 1) viral vector components; and 2) a nuclease, wherein the nuclease is expressed in the production cell and secreted in cell culture thereby degrading residual nucleic acid during viral vector production.

4. A method of producing a viral vector, the method comprising contacting 1) a viral vector production cell expressing viral vector components with 2) a nuclease helper cell expressing a nuclease, wherein the nuclease is expressed in the helper cell and secreted in co-culture of the production cell with the helper cell thereby degrading residual nucleic acid during viral vector production.

5. A method of producing a viral vector, the method comprising contacting 1) a viral vector production cell expressing viral vector components with 2) a liquid feed from nuclease helper cell expressing a nuclease, wherein the nuclease is expressed in the cell and secreted in cell culture thereby degrading residual nucleic acid during viral vector production.

6. The viral vector production system or method of any of claims 1-5, wherein the cell culture comprises a volume of at least about 5 litres of medium.

7. The viral vector production system or method of any of paragraphs 1-6, wherein the cell culture comprises a volume of at least about 50 litres of medium.

8. The viral vector production system or method of any one of paragraphs 1 to 7, wherein the production cell is a HEK293 cell, or a derivative thereof.

9. The viral vector production system or method of paragraph 8, wherein the HEK293 production cell is a HEK293T cell.

10. The viral vector production system or method of any of paragraphs 1-9, wherein the viral vector components comprise a nucleotide of interest (NOI).

11. The viral vector production system or method of any of paragraphs 1-10, wherein the viral vector components are retroviral vector components.

12. A production cell for producing viral vectors comprising nucleic acid sequences encoding: 1) viral vector components; and 2) a nuclease, wherein the nuclease is expressed in the viral vector production cell and secreted in cell culture thereby degrading residual nucleic acid during viral vector production, and wherein the production cell is a eukaryotic production cell.

13. A production cell for producing viral vectors comprising nucleic acid sequences encoding: 1) viral vector components; and 2) a nuclease fusion protein, wherein the nuclease fusion protein comprises an exonuclease domain fused to an endonuclease domain, and wherein the nuclease fusion protein is expressed in the viral vector production cell and secreted in cell culture thereby degrading residual nucleic acid during viral vector production, and wherein the production cell is a eukaryotic production cell.

14. A cell culture device comprising a viral vector production system comprising a viral vector production cell comprising nucleic acid sequences encoding: 1) viral vector components; and 2) a nuclease, wherein the nuclease is expressed in the production cell and secreted in cell culture thereby degrading residual nucleic acid during viral vector production.

15. A modified nuclease having increased cell-retention and/or or cell-association that is expressed through the secretory pathway of a eukaryotic cell, wherein the modified nuclease comprises a retention signal at its C-terminus.

16. The viral vector production system, method, production cell, cell culture device, variant of a secreted nuclease, modified nuclease or eukaryotic cell of any of the previous paragraphs wherein secreted nuclease activity within the viral vector production culture is at least about 1 unit per mL of equivalent Benzonase® nuclease activity as determinable by the assay presented as Assay 1 herein.

17. The viral vector production system, method, production cell, cell culture device, variant of a secreted nuclease, modified nuclease or eukaryotic cell of any of the previous paragraphs wherein secreted nuclease activity within the viral vector production culture is at least about 10 units per mL of equivalent Benzonase® nuclease activity as determinable by the assay presented as Assay 1 herein.

18. The viral vector production system, method, production cell, cell culture device, variant of a secreted nuclease, modified nuclease or eukaryotic cell of any of the previous paragraphs wherein secreted nuclease activity within the viral vector production culture is at least about 100 units per mL of equivalent Benzonase® nuclease activity as determinable by the assay presented as Assay 1 herein.

19. The viral vector production system, method, production cell, cell culture device, variant of a secreted nuclease, modified nuclease or eukaryotic cell of paragraphs 16-18 wherein the nuclease activity is supplied by the viral vector production cell.

20. The viral vector production system, method, production cell, cell culture device, variant of a secreted nuclease, modified nuclease or eukaryotic cell of paragraphs 16-18 wherein the nuclease activity is supplied by a nuclease helper cell.

21. A nuclease helper cell wherein secreted nuclease activity within the helper cell culture is at least about 10 unit per mL of equivalent Benzonase® nuclease activity as determinable by the assay presented as Assay 1 herein.

22. A nuclease helper cell wherein secreted nuclease activity within the helper cell culture is at least about 100 unit per mL of equivalent Benzonase® nuclease activity as determinable by the assay presented as Assay 1 herein.

23. A nuclease helper cell wherein secreted nuclease activity within the helper cell culture is at least about 2000 units per mL of equivalent Benzonase® nuclease activity as determinable by the assay presented as Assay 1 herein.

24. The viral vector production system, method, production cell, cell culture device, variant of a secreted nuclease, modified nuclease or eukaryotic cell of any of the previous paragraphs wherein the nuclease expression cassette utilizes a strong promoter, preferably the CMV promoter.

25. The viral vector production system, method, production cell, cell culture device, variant of a secreted nuclease, modified nuclease or eukaryotic cell of any of the previous paragraphs utilizing a nuclease helper cell, wherein the nuclease helper cells are added to the main viral vector production vessel at or after the point of sodium butyrate supplementation.

26. The viral vector production system, method, production cell, cell culture device, variant of a secreted nuclease, modified nuclease or eukaryotic cell of any of the previous paragraphs, wherein the expression of nuclease within viral vector production vessels enables the use of downstream processing without the requirement for further nuclease treatment.

These and other aspects of the present invention will now be described by way of numbered paragraphs 1-82 presented as numbered paragraphs set 2.

### Numbered Paragraphs Set 2:

1. A viral vector production system comprising a viral vector production cell comprising nucleic acid sequences encoding: 1) viral vector components; and 2) a nuclease, wherein the nuclease is expressed in the production cell and secreted in cell culture thereby degrading residual nucleic acid during viral vector production.

2. A viral vector production system comprising: 1) a viral vector production cell comprising nucleic acid sequences encoding viral vector components; and 2) a nuclease helper cell comprising a nucleic acid sequence encoding a nuclease, wherein the nuclease is expressed and secreted in co-culture of the production cell of 1) and the helper cell of 2), thereby degrading residual nucleic acid during viral vector production.

3. A method of producing a viral vector, the method comprising, transfecting (sometimes referred to as contacting) a viral vector production cell with nucleic acid sequences encoding: 1) viral vector components; and 2) a nuclease, wherein the nuclease is expressed in the production cell and secreted in cell culture thereby degrading residual nucleic acid during viral vector production.

4. A method of producing a viral vector, the method comprising contacting 1) a viral vector production cell expressing viral vector components with 2) a nuclease helper cell expressing a nuclease, wherein the nuclease is expressed in the helper cell and secreted in co-culture of the production cell with the helper cell thereby degrading residual nucleic acid during viral vector production.

5. A method of producing a viral vector, the method comprising contacting 1) a viral vector production cell expressing viral vector components with 2) a liquid feed from nuclease helper cell expressing a nuclease, wherein the nuclease is expressed in the cell and secreted in cell culture thereby degrading residual nucleic acid during viral vector production.

6. In an improved method of producing a viral vector, the improvement comprising introducing nucleic acid sequences into a viral vector production cell, wherein the nucleic acid sequences encode: 1) viral vector components; and 2) a nuclease, wherein the nuclease is expressed in the production cell and secreted in cell culture thereby degrading residual nucleic acid during viral vector production.

7. In an improved method of producing a viral vector, the improvement comprising contacting in co-culture a viral vector production cell expressing viral vector components with a nuclease helper cell expressing a nuclease, wherein the nuclease is expressed in the production cell and secreted in cell culture thereby degrading residual nucleic acid during viral vector production.

8. The viral vector production system or method of any of paragraphs 1-7, wherein cell culture is maintained in a pH range of 6.5 to 7.2.

9. The viral vector production system or method of any of paragraphs 1-7, wherein the extracellular nuclease is selected from the group consisting of smNucA, VsEndA, VcEndA and BacNucB.

10. The viral vector production system or method of any of paragraphs 1-7, wherein the nuclease is an extracellular nuclease.

11. The viral vector production system or method of any of paragraphs 1-7, wherein the nuclease is a sugar-non-specific nuclease.

12. The viral vector production system or method of any of paragraphs 1-7, wherein the nuclease comprises Serratia marcescens Nuclease A of SEQ ID NO: 3 or a variant thereof having at least 90% sequence identity to SEQ ID NO: 3.

13. The viral vector production system or method of any of paragraphs 1-7, wherein the nuclease comprises Vibrio cholerae Endonuclease I of SEQ ID NO: 1 or a variant thereof having at least 90% amino acid identity to SEQ ID NO: 1.

14. The viral vector production system or method of paragraph 13, wherein the nuclease is a nuclease variant selected from the group consisting VcEndA-12glc of SEQ ID NO: 5, VcEndA-123glc of SEQ ID NO: 6, VcEndA-124glc of SEQ ID NO: 7, VcEndA-134glc of SEQ ID NO: 8, VcEndA-13glc of SEQ ID NO: 9, VcEndA-14glc of SEQ ID NO: 10, and VcEndA-1glc of SEQ ID NO: 11.

15. The viral vector production system or method of paragraph 13, wherein the nuclease is nuclease variant VcEndA-1glc of SEQ ID NO: 11.

16. The viral vector production system or method of any of paragraphs 1-7, wherein the nuclease comprises a salt-active nuclease.

17. The viral vector production system or method of paragraph 16, wherein the salt-active nuclease comprises Vibrio salmonicida Endonuclease I of SEQ ID NO: 2 or a variant thereof having at least 90% amino acid identity to SEQ ID NO: 2

18. The viral vector production system or method of any of paragraphs 1-7, wherein the nuclease comprises BacNucB of SEQ ID NO: 4, or a variant thereof having at least 90% amino acid identity to SEQ ID NO: 4.

19. The viral vector production system or method of any of paragraphs 1-18, wherein the nuclease comprises a native or non-native N-terminal secretory signal.

20. The viral vector production system or method of any of paragraphs 1-18, wherein the nucleic acid sequences encoding a nuclease are sequence-optimised to remove potential splice sites and/or unstable elements.

21. The viral vector production system or method of any of paragraphs 1-18, wherein the nucleic acid sequences encoding a nuclease are codon-optimised for expression in the production cell.

22. The viral vector production system or method of any of paragraphs 1-21, wherein the production system and/or methods further comprise one or more additional nucleases.

23. The viral vector production system or method of paragraph 22, wherein the nuclease is fused to at least one of the one or more additional nucleases.

24. The viral vector production system or method of any of paragraphs 1-23, wherein the cell culture comprises a volume of at least about 5 litres of medium.

25. The viral vector production system or method of any of paragraphs 1-24, wherein the cells are in suspension or adherent.

26. The viral vector production system or method of any of paragraphs 1-25, wherein the cell culture is serum-free.

27. The viral vector production system or method of any of paragraphs 1-26, wherein the production cell is a eukaryotic cell.

28. The viral vector production system or method of paragraph 27, wherein the production cell is a mammalian cell.

29. The viral vector production system or method of paragraph 28, wherein the production cell is a human production cell.

30. The viral vector production system or method of any one of paragraphs 1-29, wherein the production cell is a HEK293 cell, or a derivative thereof.

31. The viral vector production system or method of paragraph 30, wherein the HEK293 production cell is a HEK293T cell.

32. The viral vector production system or method of any of paragraphs 1-31, wherein the viral vector components comprise a nucleotide of interest (NOI).

33. The viral vector production system or method of any of paragraphs 1-32, wherein the viral vector components are retroviral vector components.

34. The viral vector production system or method of paragraph 33, wherein the retroviral vector components are lentiviral vector components.

35. The viral vector production system or method of paragraph 34, wherein the viral vector components comprise i) gag-pol; ii) env; iii) optionally the RNA genome of a retroviral vector; and iv) optional rev, or a functional substitute thereof.

36. The viral vector production system or method of paragraph 35, wherein at least two of the nucleic acid sequences are modular constructs encoding the viral vector components located at the same genetic locus.

37. The viral vector production system or method of paragraph 35, wherein at least two of the nucleic acid sequences are modular constructs encoding the viral vector components in reverse and/or alternating orientations.

38. The viral vector production system or method of paragraph 35, wherein at least two of the nucleic acid sequences are modular constructs encoding gag-pol and/or env, wherein the modular constructs are associated with at least one regulator element.

39. The viral vector production system or method of paragraphs 35-38, wherein the env is a VSV-G env.

40. The viral vector production system or method of any of paragraphs 1-39, wherein the nuclease is expressed and secreted from the cell after transient transfection.

41. The viral vector production system or method of any of paragraphs 1-39, wherein the nuclease is expressed and secreted from the cell after stable integration of the cell.

42. The viral vector production system or method of paragraph 41, wherein the expression of the nuclease is inducible or conditional, and wherein the nucleic acid encoding the nuclease comprises an inducible or conditional promoter or regulatory element.

43. A production cell for producing viral vectors comprising nucleic acid sequences encoding: 1) viral vector components; and 2) a nuclease, wherein the nuclease is expressed in the viral vector production cell and secreted in cell culture thereby degrading residual nucleic acid during viral vector production, and wherein the production cell is a eukaryotic production cell.

44. The cell according to paragraph 43, wherein the nuclease is an endonuclease.

45. The cell according to paragraph 44, further comprising a nucleic acid sequence encoding an exonuclease.

46. The cell according to paragraph 45, wherein the endonuclease is fused to the exonuclease.

47. The cell according to paragraph 43, wherein the nuclease is an exonuclease.

48. The cell according to paragraph 48, wherein the exonuclease is fused to the endonuclease.

49. The cell of paragraph 43, wherein the cell is a transient production cell.

50. The cell of paragraph 43, wherein the cell is a stable production cell.

51. The cell of paragraph 50, wherein nuclease expression and secretion is inducible expression and secretion in the stable production cell.

52. A production cell for producing viral vectors comprising nucleic acid sequences encoding: 1) viral vector components; and 2) a nuclease fusion protein, wherein the nuclease fusion protein comprises an exonuclease domain fused to an endonuclease domain, and wherein the nuclease fusion protein is expressed in the viral vector production cell and secreted in cell culture thereby degrading residual nucleic acid during viral vector production, and wherein the production cell is a eukaryotic production cell.

53. The cell of paragraph 52, wherein the endonuclease is VcEndA or a variant thereof.

54. The cell of paragraph 52, wherein the endonuclease is VcEndA-1glc of SEQ ID NO: 11.

55. A cell culture device comprising a viral vector production system comprising a viral vector production cell comprising nucleic acid sequences encoding: 1) viral vector components; and 2) a nuclease, wherein the nuclease is expressed in the production cell and secreted in cell culture thereby degrading residual nucleic acid during viral vector production.

56. The cell culture device of paragraph 46, which is a stir-tank bioreactor or a wave-bag or iCELLis® bioreactor.

57. A variant of a secreted nuclease capable of degrading residual nucleic acid during viral vector production, said variant comprising the amino acid sequence of SEQ ID NO: 11.

58. A modified nuclease having increased cell-retention and/or or cell-association that is expressed through the secretory pathway of a eukaryotic cell, wherein the modified nuclease comprises a retention signal at its C-terminus.

59. The modified nuclease of paragraph 58, wherein the modified nuclease localizes to the endoplasmic reticulum (ER) and/or the golgi compartments thereby resulting in increased cell retention compared to cell retention of a corresponding unmodified nuclease.

60. The modified nuclease of paragraph 58, wherein the modified nuclease is bound to ER receptors of the ER retention-defective complementation group.

61. The modified nuclease of paragraph 58, wherein the retention signal is at its C-terminus of consensus [KRHQSA]-[DENQ]-E-L (SEQ ID NO: 19) or KKXX 62. The modified nuclease of paragraph 58, wherein the retention signal is at its C-terminus of consensus KDEL (SEQ ID NO: 21).

63. A eukaryotic cell expressing the modified nuclease of paragraph 58.

64. A viral vector production system comprising the cell of paragraph 63.

65. A production cell for producing viral vectors comprising a viral production cell as defined in any one of paragraphs 1 to 42.

66. A production cell according to paragraph 43 or any paragraph dependent thereon comprising a viral production cell as defined in any one of paragraphs 1 to 42.

67. A production cell according to paragraph 52 or any paragraph dependent thereon comprising a viral production cell as defined in any one of paragraphs 1 to 42.

68. A cell culture device comprising a production cell as defined in any one of paragraphs 1 to 42.

69. A cell culture device comprising a production cell as defined in paragraph 43 or any paragraph dependent thereon.

70. A cell culture device comprising a production cell as defined in paragraph 52 or any paragraph dependent thereon.

71. The viral vector production system or method according to any one of paragraphs 1 to 42 comprising a nuclease as defined in any one of paragraphs 57-62.

72. The viral vector production system, method, production cell, cell culture device, variant of a secreted nuclease, modified nuclease or eukaryotic cell of any of the previous paragraphs wherein secreted nuclease activity within the viral vector production culture is at least about 1 unit per mL of equivalent Benzonase® nuclease activity as determinable by the assay presented as Assay 1 herein.

73. The viral vector production system, method, production cell, cell culture device, variant of a secreted nuclease, modified nuclease or eukaryotic cell of any of the previous paragraphs wherein secreted nuclease activity within the viral vector production culture is at least about 10 units per mL of equivalent Benzonase® nuclease activity as determinable by the assay presented as Assay 1 herein.

74. The viral vector production system, method, production cell, cell culture device, variant of a secreted nuclease, modified nuclease or eukaryotic cell of any of the previous paragraphs wherein secreted nuclease activity within the viral vector production culture is at least about 100 units per mL of equivalent Benzonase® nuclease activity as determinable by the assay presented as Assay 1 herein.

75. The viral vector production system, method, production cell, cell culture device, variant of a secreted nuclease, modified nuclease or eukaryotic cell of paragraphs 72-74 wherein the nuclease activity is supplied by the viral vector production cell

76. The viral vector production system, method, production cell, cell culture device, variant of a secreted nuclease, modified nuclease or eukaryotic cell of paragraphs 72-74 wherein the nuclease activity is supplied by a nuclease helper cell

77. A nuclease helper cell wherein secreted nuclease activity within the helper cell culture is at least about 10 unit per mL of equivalent Benzonase® nuclease activity as determinable by the assay presented as Assay 1 herein.

78. A nuclease helper cell wherein secreted nuclease activity within the helper cell culture is at least about 100 unit per mL of equivalent Benzonase® nuclease activity as determinable by the assay presented as Assay 1 herein.

79. A nuclease helper cell wherein secreted nuclease activity within the helper cell culture is at least about 2000 units per mL of equivalent Benzonase® nuclease activity as determinable by the assay presented as Assay 1 herein.

80. The viral vector production system, method, production cell, cell culture device, variant of a secreted nuclease, modified nuclease or eukaryotic cell of any of the previous paragraphs wherein the nuclease expression cassette utilizes a strong promoter such as CMV.

81. The viral vector production system, method, production cell, cell culture device, variant of a secreted nuclease, modified nuclease or eukaryotic cell of any of the previous paragraphs utilizing a nuclease helper cell, wherein the nuclease helper cells are added to the main viral vector production vessel at or after the point of sodium butyrate supplementation.

82. The viral vector production system, method, production cell, cell culture device, variant of a secreted nuclease, modified nuclease or eukaryotic cell of any of the previous paragraphs, wherein the expression of nuclease within viral vector production vessels enables the use of downstream processing without the requirement for further nuclease treatment.

Various preferred features and embodiments of the invention will now be described by way of non-limiting examples.

### EXAMPLES

### Example 1: Expression Constructs Encoding Widely Divergent Nucleases for the Reduction of Residual DNA During Viral Vector Production Cell Culture

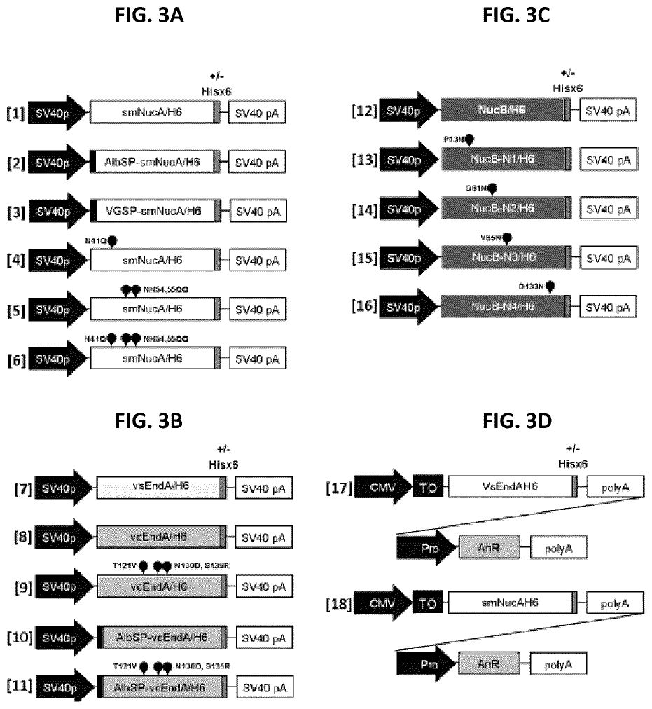

Expression plasmids were constructed for Serratia marcescens Endonuclease A (SmNucA), VsEndA and BacNucB according to FIG. 3A-3D. All constructs contained the SV40 promoter and polyadenylation signal. ORFs were codon-optimised (Homo sapiens) and were 6×Histidine (SEQ ID NO: 27) tagged at their C-teminus (H6). SmNucA expression plasmids encoded wild type smNucA with its own bacterial secretory sequence or had its bacterial secretory sequence replaced with that of human Albumin or VSV-G, or additionally included NxS/T sequon mutations at the stated positions. VsEndA or VcEndA expression plasmids encoded wild type nuclease with its own bacterial secretory sequence or had its bacterial secretory seuquence replaced with that of human Albumin, or additionally included NxS/T sequon mutations at T121V, N130D, S135R. BacNucB expression plasmids encoded wild type BacNucB with its own bacterial secretory sequence or additionally contained y>N mutations at the stated positions, resulting in NxS/T sequons.

### Example 2: Demonstration of Expression and Secretion of a Nuclease from HEK293T Cells Correlated with Reduced Residual DNA in Culture Media

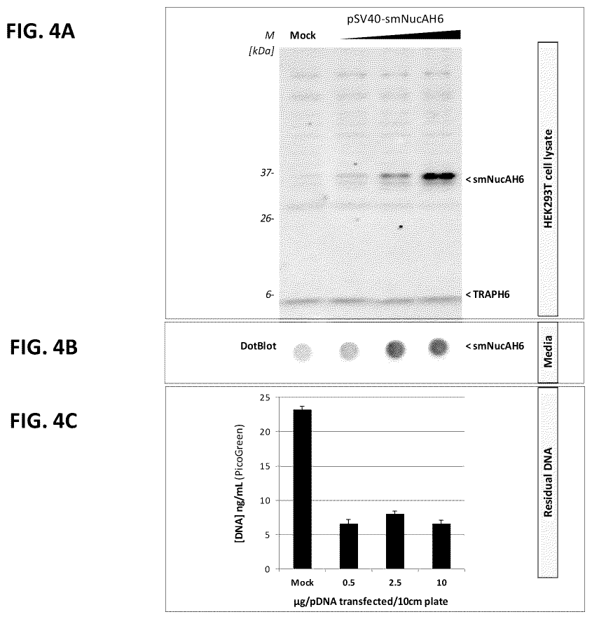

HEK293T cells were transfected with a fixed amount of plasmid DNA (total g) using different ratios of stuffer DNA (pBluescript) and pSV40-smNucAH6, which encodes SmNucA fused with a C-terminal His-tag to allow for protein detection. Cell lysates and culture media were analysed by immunoblotting to the His-tag (an endogenous His-tagged protein ‘TRAPH6’ was used as a loading control), which demonstrated that SmNucAH6 was expressed and secreted in the cultures in a dose-dependent manner. Clarified culture supernatants were analysed for residual DNA by PicoGreen assay, which demonstrated that reduction in DNA detection only occurred in the presence of SmNucAH6 in the media. Results are shown in FIG. 4A-4C.

### Example 3: C-Terminal Histidine Tagged and Untagged Secreted Nucleases During Production of Lentiviral Vector Production

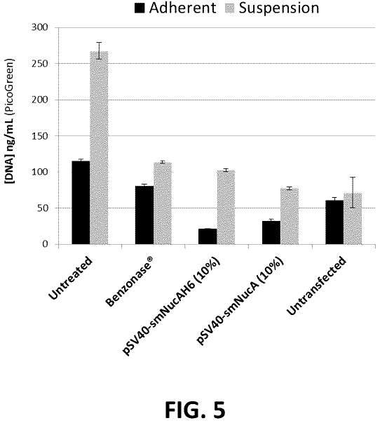

HIV-1 based lentiviral vectors were produced by transient co-transfection of secreted nuclease plasmids into either adherent or suspension HEK293T cells at 10% input of total pDNA. Lentiviral vector harvests were left ‘untreated’ or were treated with Benzonase® for 1 hour prior to clarification. Secreted nuclease cultures were not treated with Benzonase®. Filtered culture media was analysed by PicoGreen assay. Secreted nucleases produced equivalent or better DNA reduction than Benzonase® in culture media. Results are shown in FIG. 5.

### Example 4: Eukaryotic ER Signal Peptides can Replace Bacterial Secretory Peptide of smNucA

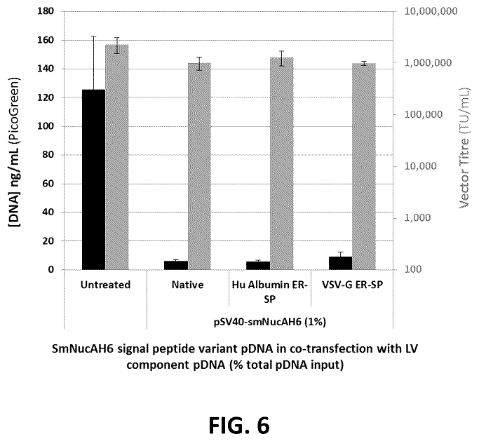

HIV-1 based lentiviral vectors (HIV-CMV-GFP) were produced by transient co-transfection of secreted nuclease plasmids into adherent HEK293T cells at 1% input of total pDNA. The SmNucA ORF contained either its native bacterial secretory signal (Native) or the human Albumin ER signal peptide (Hu Albumin ER-SP) or the VSV-G ER signal peptide (VSV-G ER-SP). None of the lentiviral vector harvests were treated with Benzonase®. Filtered culture media was analysed by PicoGreen assay (black bars) and lentiviral vectors were titrated by transduction of HEK293T cells followed by flow cytometry. Results are shown in FIG. 6.

### Example 5: Divergent Secreted Nucleases During Generation of Lentiviral Vectors in Adherent HEK293T Cell Cultures During Transient Transfection of Vector Components

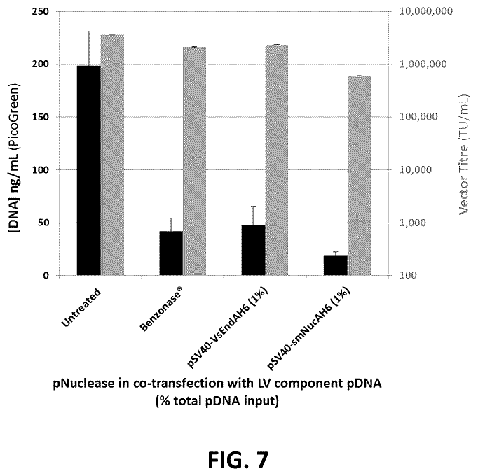

HIV-1 based lentiviral vectors (HIV-CMV-GFP) were produced by transient co-transfection of either SmNucAH6 or VsEndAH6 nuclease plasmids into adherent HEK293 Ts at 1% input of total pdNA. Lentiviral vector harvests were left ‘untreated’ or were treated with Benzonase® for 1 hour prior to vector harvest. Secreted nuclease cultures were not treated with Benzonase®. Filtered culture media was analysed by PicoGreen assay (black bars) and LVs titrated by transduction of HEK293 Ts followed by flow cytometry. Results are shown in FIG. 7.

### Example 6: Divergent Secreted Nucleases During Generation of Lentiviral Vectors in Adherent Stable Producer Cell Line Cultures

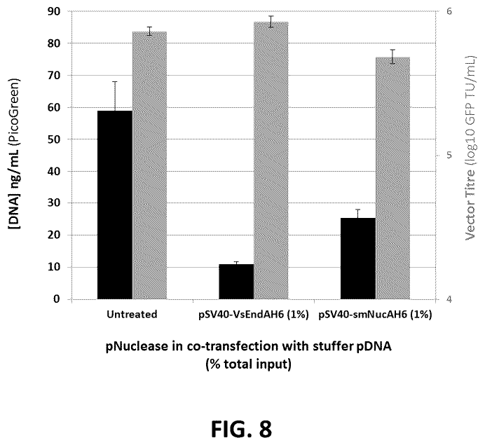

HIV-1 based lentiviral vectors (HIV-CMV-GFP) were produced by transient transfection of a producer cell line with either SmNucAH6 or VsEndAH6 nuclease plasmids at 1% input of total pDNA (pBluescript was added as standard stuffer). Approximately 18 hours post-transfection, cultures were induced by addition of doxycycline (1000 ng/ml final concentration), which resulted in expression of packaging components driven by the CMV-tetO promoter (GFP genome constitutively expressed). Filtered culture media was analysed by PicoGreen assay (black bars) and LVs titrated by transduction of HEK293 Ts followed by flow cytometry. Results are shown in FIG. 8.

### Example 7: Divergent Secreted Nucleases During Generation of Lentiviral Vectors in Suspension Cell Cultures

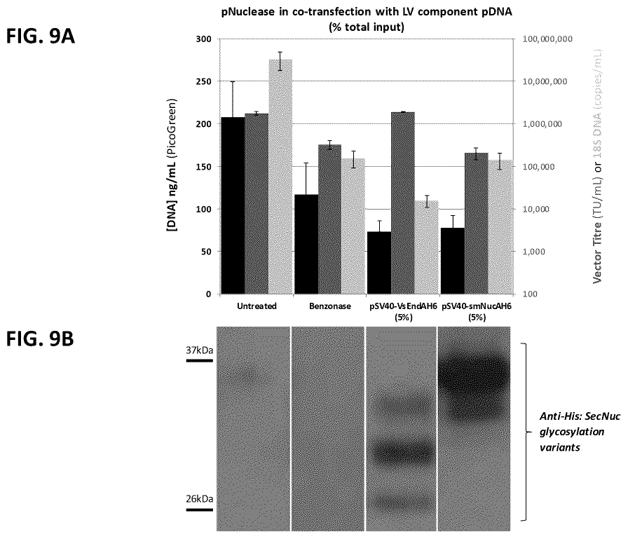

HIV-1 based lentiviral vectors (HIV-CMV-GFP) were produced in HEK293T cells in suspension. The cells were transiently co-transfection with either SmNucAH6 or VsEndAH6 nuclease plasmids at 5% input of total pDNA. Lentiviral vector harvests were left ‘untreated’ or were treated with Benzonase® for 1 hour prior to vector harvest. Secreted nuclease cultures were not treated with Benzonase®. Filtered culture media was analysed by PicoGreen assay (black bars) and purified total DNA was analysed for production cell DNA by qPCR against 18S DNA (light grey bars). Lentiviral vector supernatant was titrated by transduction of HEK293 Ts followed by flow cytometry (dark grey bars). Concentrated vector supernatant was subjected to SDS-PAGE and immunoblotting using anti-His Tag antibody to detect secreted nuclease proteins. Results are shown in FIG. 9A-9B.

### Example 8: Clearance of Residual Plasmid DNA in Lentiviral Vector Production Suspension, Serum-Free Cell Cultures Using BacNucB and BacNucB Variants

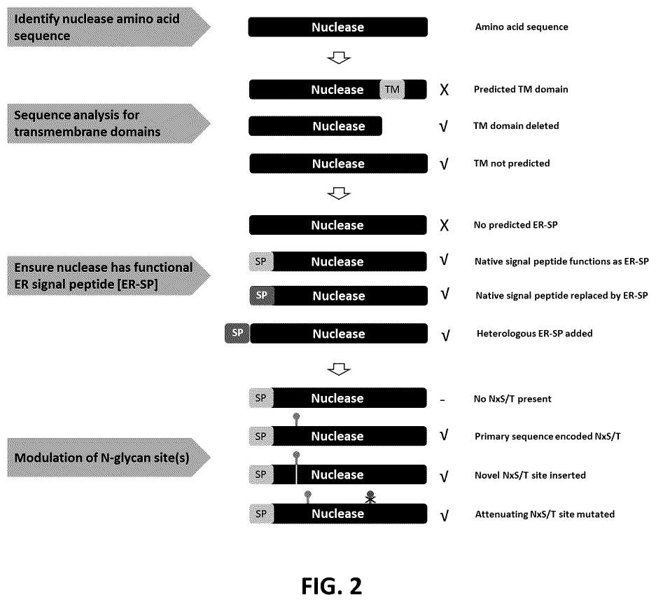

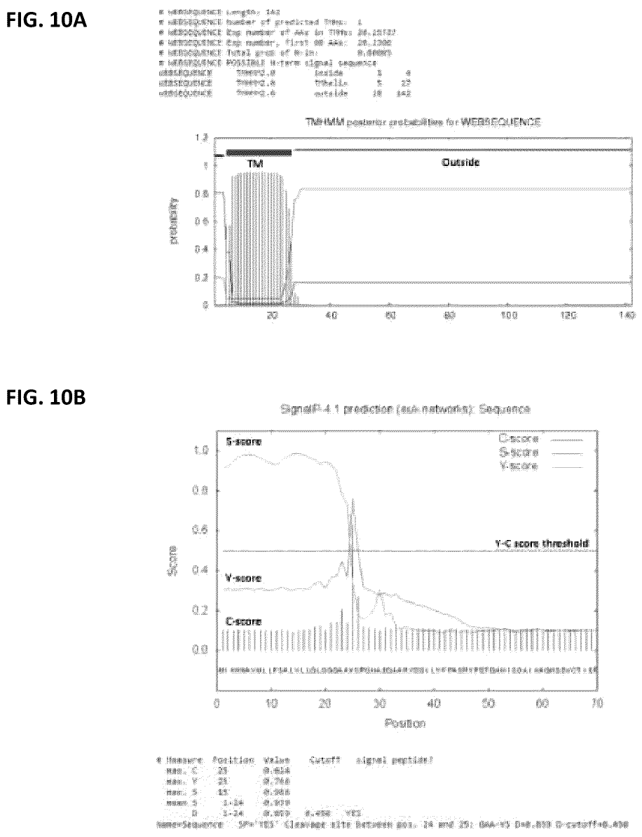

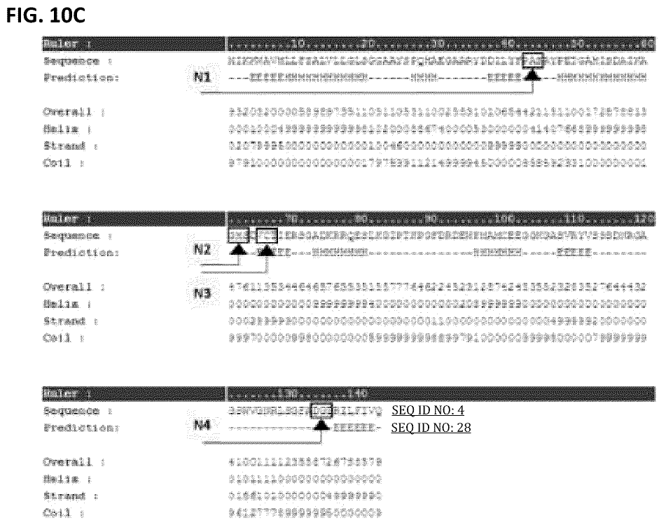

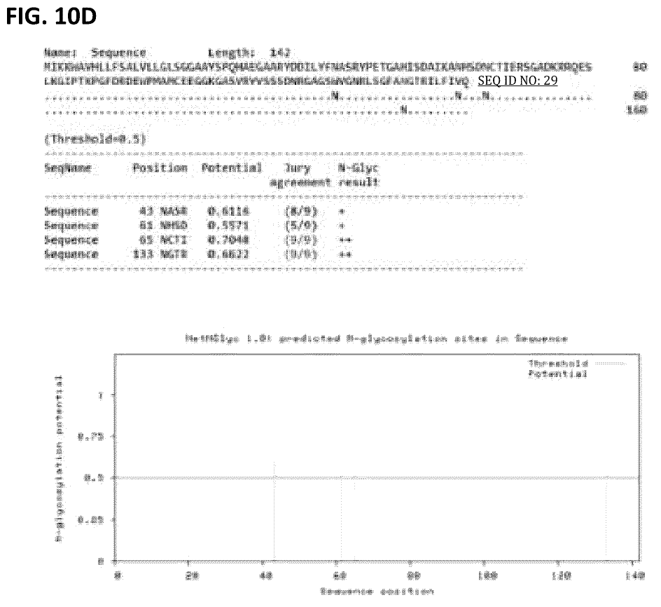

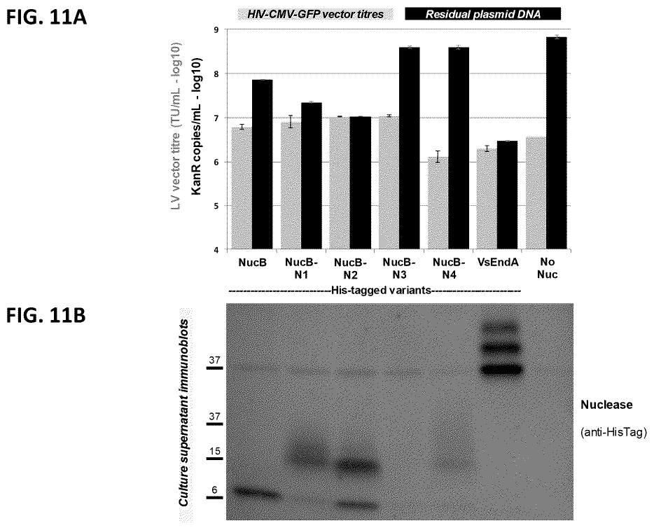

Bacillus species BacNucB protein (SEQ ID No: 4) does not comprise any NxS/T sequons and therefore cannot be N-glycosylated when targeted to the secretory pathway in eukaryotic cells. It was therefore unknown if BacNucB could be used effectively in the clearance of residual DNA from vector production cultures, as lack of N-glycosylation might hinder secretion levels. The work flow outlined in FIG. 2 was applied to the primary sequence of BacNucB (SEQ ID NO: 4). The sequence was analysed and TMHMM (CBS) was used to determine that there were no TM (transmembrane) domains on the C-terminal side of the signal peptide (which is predicted to be a TM). Analysis of BacNucB by SignalP (CBS) confirmed that the bacterial secretory peptide is predicted to function as an ER signal peptide, cleavage of which is predicted to generate the complete mature nuclease. Further analysis by YAPIN (Centre for integrative Bioinformatics VU (IBIVU)) predicted regions of alpha-helices, beta-strands and coils which pinpoints regions where NxS/T motifs could be introduced into the BacNucB protein sequence without disrupting smaller folds. As such, the following 4 variants of BacNucB were identified: P43N (sequon=NAS) is variant ‘N1’, G61N (sequon=NHS) is ‘N2’, V65N (sequon=NCT) is variant ‘N3’, and D133N (sequon=NGT) is variant ‘N4’. The primary sequence of BacNucB with the ‘N’ mutations at 43, 61, 65, and 133 were submitted to NetNGlyc (CBS) for analysis. All four NxS/t sequons had scores above the threshold (0.5), with N65 and N133 variants achieving the highest probability of improved functionality. Results are shown in FIG. 10A-10D.

Based on the above analysis, four variants of BacNucB (N1, N2, N3, and N4) were generated by inserting NxS/T sequons into its primary amino acid sequence by mutating the Y residue to an N, at four YxS/T sites within the native protein sequence. This was done at positions that are predicted not to participate in secondary folds in order to maximize the likelihood that potential N-glycosylation would not affect protein folding.

All BacNucB expression plasmids were His-tagged at their C-termini for ease of detection. Suspension, serum-free HEK293T cells were transfected with HIV-CMV-GFP vector components and nuclease plasmids at 5% input of total pDNA. Purified total DNA from vector harvests was analysed for residual plasmid by qPCR against KanR sequences (black bars). Lentiviral vector supernatant was titrated by transduction of HEK293T cells followed by flow cytometry (grey bars). Concentrated vector supernatant was subjected to SDS-PAGE and immunoblotting using anti-HisTag antibody to detect secreted nuclease proteins. The data demonstrates that residual plasmid DNA is only modestly cleared by BacNucBH6 but that two of the N-glycan variants showed enhanced clearance of residual plasmid DNA. Note that the expression plasmid encoding VsEndAH6 was superior to all expression plasmids encoding BacNucB in terms of residual DNA clearance.

### Example 9: Clearance of Residual Plasmid DNA in Lentiviral Vector Production in Suspension, Serum-Free Cell Cultures Using VcEndA Variants

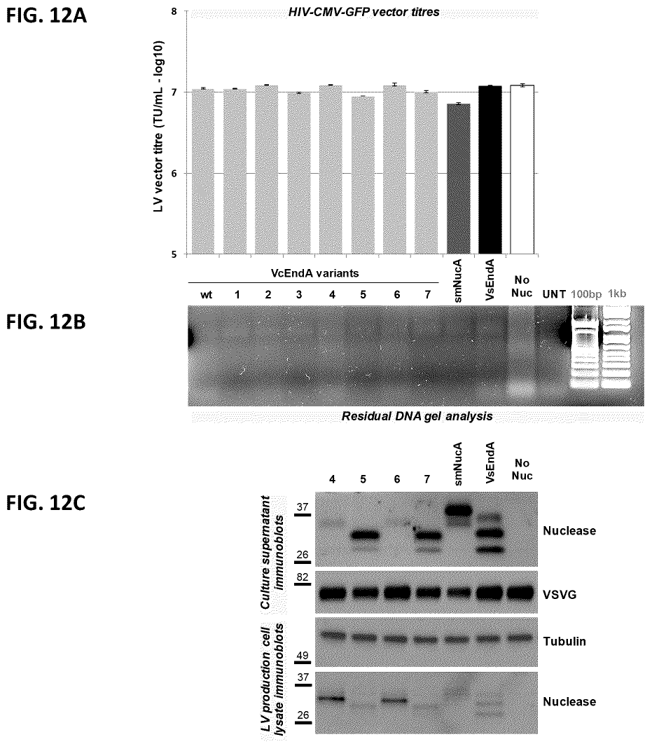

VcEndA and VsEndA share 68% identity at their amino acid sequence (secreted forms). VcEndA has been shown to have optimal activity between 150 and 200 mM salt (i.e., close to salt concentration in typical eukaryotic cell cultures) and is more active at pH6.5 and pH7 than VsEndA. Analysis of VcEndA (as per the workflow shown in FIG. 2) reveals three of its four NxS/T sequons are not present in VsEndA. ‘1glc’ VcEndA variants were generated (as shown in FIG. 3A-3D) by ablating these sequons at all three positions. Additional VcEndA variants were generated with the human Albumin ER signal peptide, as well as optional C-terminal His-tags. (Key: wt=VcEndA, 1=VcEndA-1glc, 2=AlbVcEndA, 3=AlbVcEndA-1glyc; 4, 5, 6, 7=His-tagged versions of wt, 1, 2, 3 respectively. HIV-CMV-GFP vector was made in suspension, serum-free cultures co-transfected with nuclease plasmids at 5% total input pDNA. Lentiviral vector supernatant was titrated by transduction of HEK 293T cells followed by flow cytometry (bars). Concentrated vector supernatant (VcEndA variants 4-7) were subjected to SDS-PAGE/immunoblotting using anti-HisTag antibody to detect secreted nuclease proteins, as well as VSV-G (lentiriviral vector virions). Post-production cell lysates were also probed for nuclease expression. Residual DNA from vector supernatants was visualized by agarose-electrophoresis. VcEndA (wt) is poorly secreted into the culture media but the ‘1glc’ variants are efficiently secreted, leading to efficient clearance of residual DNA. Results are shown in FIG. 12A-12C.

### Example 10: Degradation of Residual DNA from Lentiviral Vector Production Bioreactors at 0.5 L Scale

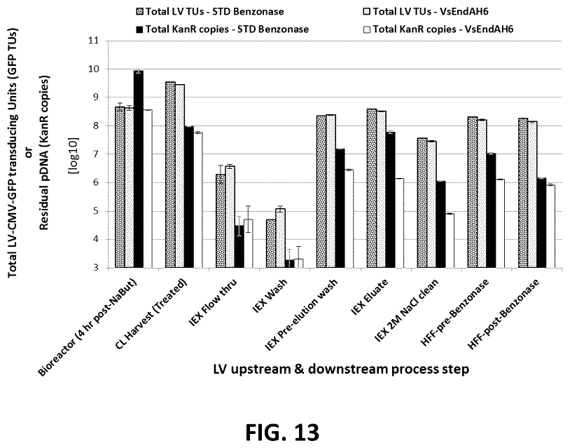

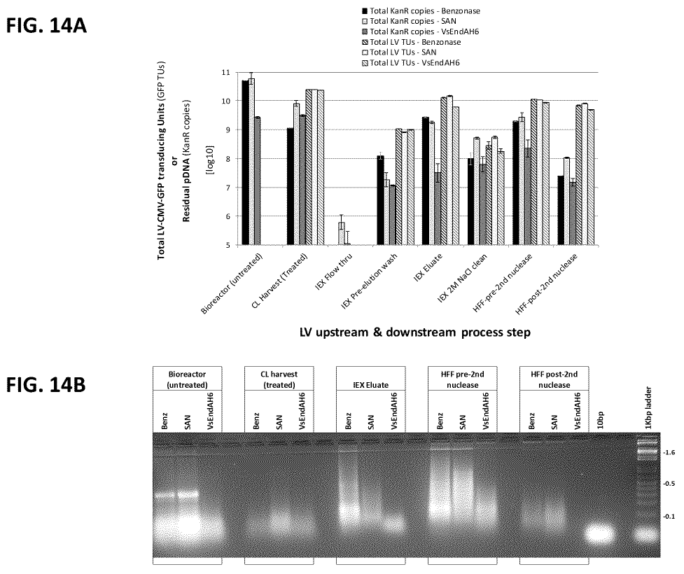

Suspension, serum-free adapted HEK293T cells were seeded into six 0.5 litre (L) bioreactors and triplicate bioreactors transfected with HIV-1 based lentiviral vector components (GFP expressing genome) together with either pBluescript (referred as STD—Benzonase® in FIG. 13) or pSV40-VsEndAH6 at 5% total plasmid input. Twenty hours post-transfection, sodium butyrate (NaBut) was added to all bioreactors at a final concentration of 10 mM. Four hours after NaBut induction, samples were taken (Bioreactor [4 hr post-NaBut]). One hour prior to vector harvest, the pBluescript-co-transfected bioreactors were inoculated with Benzonase® at 5 U/ml final concentration. The pSV40-VsEndAH6-co-transfected bioreactors were left untreated. The bulk harvests from the triplicate bioreactors were then pooled to generated ˜1 L of harvest, which was subject to 0.45 m filtration, and samples taken for analysis (CL Harvest). This pooling was done in order to be able to perform a downstream process at a scale that allowed use of equipment/flow rates reflective of typical large scale downstream process. Material was subjected to ion-exchange (IEX) chromatography, collecting samples (IEX flow through, IEX washes, IEX elutate, and IEX column clean with 2M salt) before further processing using dia-/ultra-filtration using hollow fibre cartridges. The first step allowed buffer exchange (HFF-pre-Benzonase®) and then 400 U/ml Benzonase® was added to both the ‘standard Benzonase®’ processed vector and the VsEndAH6 treated vector, as a second nuclease step. Finally, buffer exchange occurred to remove Benzonase® (HFF-post Benzonase®). Samples were titrated to generate lentiviral vector titers (GFP/FACS; TU/ml) and residual DNA purified and subjected to plasmid copy number analysis by qPCR against KanR sequences. Total TUs and total kanR copies were generated for each step by factoring total volumes at each step. Results are shown in FIG. 13. The data show that secreted VsEndAAH6 reduces KanR detection by 10-fold in the bioreactor as early as 24 hours post-transfection, whilst lentiviral vector titers remain unaffected in the bioreactor, and during downstream processing. Whilst Benzonase®-treated harvest appeared to reduce KanR detection to a similar, albeit slightly higher level compared to VsEndAH6, KanR detection through the downstream process was lower in the VsEndAH6 material. This suggests that whiles similar numbers of KanR copies were detected in CL Harvest comparing both approaches, there was a qualitative difference between the two conditions i.e., residual DNA in the VsEndAH6 material is likely to have been smaller in size (see FIG. 14A-14B for evidence of this), and easier to remove during the downstream process.

### Example 11: Degradation of Residual DNA from Lentiviral Vector Production Bioreactors at 5 L Scale

HIV-1 based lentiviral vectors expressing GFP were produced in a similar manner as described for 0.5 L bioreactors (Example 10) however in single bioreactors at 5 L scale. SAN-HQ was also tested in parallel to Benzonase® and the secreted nuclease approach (co-transfected pSV40-VsEndAH6 at 5% of total plasmid input). FIG. 14A shows total lentiviral vector TUs and detectable plasmid (KanR) at each process step, and FIG. 14B shows concentrated samples from upstream & downstream process steps were analysed by gel electrophoresis (Ethidium bromide stain for DNA). As is shown in FIG. 13, expression plasmid-expressed secreted nuclease achieves comparable or better reduction in plasmid DNA compared to commercial nuclease (such as Benzonase®) but additionally, total DNA (which will include production cell DNA) is digested to smaller fragments which appear to be more efficiently removed by the final nuclease step on the HFF cartridge.

### Example 12: Degradation of Residual DNA within Lentiviral Vector Production Cultures Using a tetR-Regulated Nuclease Expression Plasmid

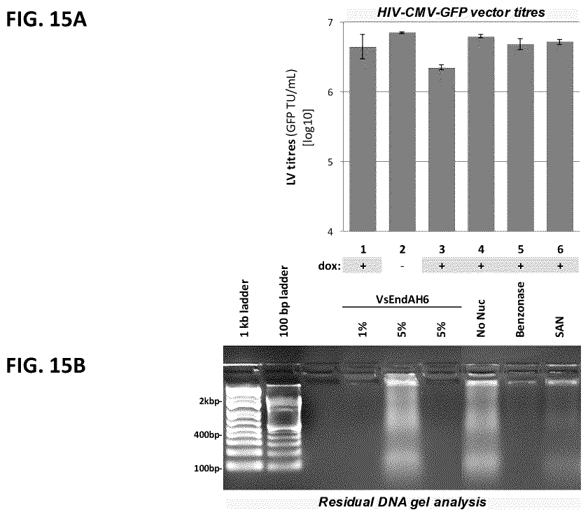

HIV-1 based vectors encoding GFP were produced by transient transfection of serum-free, suspension-adapted HEK293T.tetR cells with vector components and pCMV-TO-VsEndAH6 at either 1% or 5% input of total plasmid DNA (FIG. 15A=15B). Cultures receiving no nuclease plasmid were co-transfected with pBluescript. Approximately 20 hours post-transfection, all cultures were induced with sodium butyrate and 1000 ng/mL doxycycline (lanes 1, 3-6; to induce VsEndAH6 expression where present), except for a replicate set of cultures transfected with 5% pCMV-TO-VsEndAH6, which only received sodium butyrate (no dox; lane 2). Control cultures were either left untreated (No Nuc) or treated with Benzonase® or SAN at 5 U/mL final concentration 1 hour prior to harvest. All cultures received 2 mM MgCl₂ final concentration 1 hour prior to harvest. All conditions were carried out in triplicate. At harvest, production cells were removed by centrifugation, and supernatants filtered (0.22 μm) before titration by transduction of HEK293T cells followed by flow cytometry. Supernatants from each of the triplicate cultures were pooled, and ˜2 mL concentrated to 0.12 mL by centrifugation using 3K cut-off Amicon-15 devices, and 50% of this material loaded onto a 2% agarose gel for residual DNA electrophoresis (Ethidium Bromide). The data show that tetR-regulated nuclease expression can be employed to allow for temporal control of nuclease expression; doxycycline addition 20 hour post-transfection leads to sufficiently active levels of secreted nuclease as indicated by improved clearance of residual DNA compared to the commercial nucleases. The titres of lentiviral vectors produced in the presence of secreted nuclease were all above 1×10⁶ TU/mL, demonstrating minimal impact on the output of vector.

### Example 13: Degradation of Residual DNA within Lentiviral Vector Production Cultures by Co-Culturing with Helper Cells Expressing Secreted Nuclease

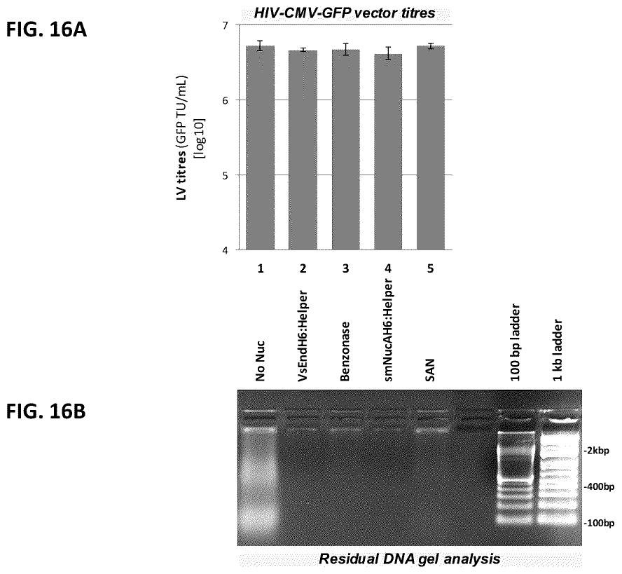

HIV-1 based vectors encoding GFP were produced by transient transfection of serum-free, suspension-adapted HEK293T-1.65S cells with vector components. In parallel and at the same scale, serum-free suspension-adapted HEK293T.tetR cells were transfected with tetR-regulated nuclease plasmids pCMV-TO-VsEndAH6 or pCMV-TO-smNucAH6. Approximately 20 hours post-transfection, cultures were inoculated with 10% of the helper Cell cultures, together with sodium butyrate and 1000 ng/mL dox, to induce nuclease expression from the helper cells. Control cultures were either left untreated (No Nuc) or treated with Benzonase® or SAN at 5 U/mL final concentration 1 hour prior to harvest. All cultures received 2 mM MgCl₂ final concentration 1 hour prior to harvest. All conditions were carried out in triplicate. At harvest, production cells were removed by centrifugation, and supernatants filtered (0.22 μm) before titration by transduction of HEK293T cells followed by flow cytometry. Supernatants from each of the triplicate cultures were pooled, and ˜2 mL concentrated to 0.12 mL by centrifugation using 3K cut-off Amicon-15 devices, and 50% of this material loaded onto a 2% agarose gel for residual DNA electrophoresis (Ethidium Bromide). Results are shown in FIG. 16A-16B. The data show that nuclease expressing helper cells can be co-cultured with vector production cells allowing sufficiently active levels of secreted nuclease as indicated by improved clearance of residual DNA compared to the commercial nucleases. The titres of lentiviral vectors produced in the presence of secreted nuclease were all above 1×10⁶ TU/mL, demonstrating no impact impact on the output of vector.

### Example 14: AAV-Based and Adenovirus-Based Vector Production Systems Expressing Secreted Nuclease for Degradation of Residual Nucleic Acid During Vector Production Requiring Freeze-Thaw Step

### A. Work Flow of Use of Secreted Nuclease in Production of AAV Vectors by Transient Transfection:

1. Seeding of production cells (e.g. HEK293) into culture vessel (adherent or suspension)
2. Transient co-transfection of vector components with nuclease plasmid:
   i. Multi-plasmid transfection of vector components pAAV genome:pRepCap:pHelper (e.g. 1:1:1 ratio), plus pNuclease cotransfected as a subfraction of total pDNA
   ii. Transfection via reagents such as Lipofectamine or PEI
3. Incubation of transfected cell cultures for several (e.g. 2-to-4) days
4. Cell lysis:
   i. Direct freeze-thawing of production cells in cultures media, or
   ii. Concentration of production cells, followed by chemical lysis or physical lysis e.g. microfluidisation/freeze-thaw
   iii. Limited incubation period to allow secreted nuclease to digest contaminating DNA
5. Filtration and optional freezing of bulk substance
6. Downstream purification/concentration e.g chromatography (ion-exchange), dia/ultra-filtration and/or Size exclusion.

### B. Work Flow of Use of Secreted Nuclease in Production of Adenoviral Vectors by Amplification in Adenovirus E1-Expressing Cells:

1. Seeding of production cells (e.g. HEK293) into culture vessel (adherent or suspension)
2. Inoculation of Adenovirus vector seed stock at MOI=1
3. Incubate cultures for 24-72 hours or until ˜50% visible cytopathic effect (cpe)
4. Cell lysis:
   i. Direct freeze-thawing of production cells in cultures media, or
   ii. Concentration of production cells, followed by chemical lysis or physical lysis e.g. microfluidisation/freeze-thaw
   iii. Limited incubation period to allow secreted nuclease to digest contaminating DNA
5. Filtration and optional freezing of bulk substance
6. Optionally, successive scaling-up of steps 1-5
7. Downstream purification/concentration e.g chromatography (ion-exchange), dia/ultra-filtration and/or Size exclusion.

### Example 15: Generation and Evaluation of VcEndA Variants Containing NxS/T Sequon Ablation Mutations to Degrade Residual DNA in Lentiviral Vector Production Serum-Free, Suspension Cultures

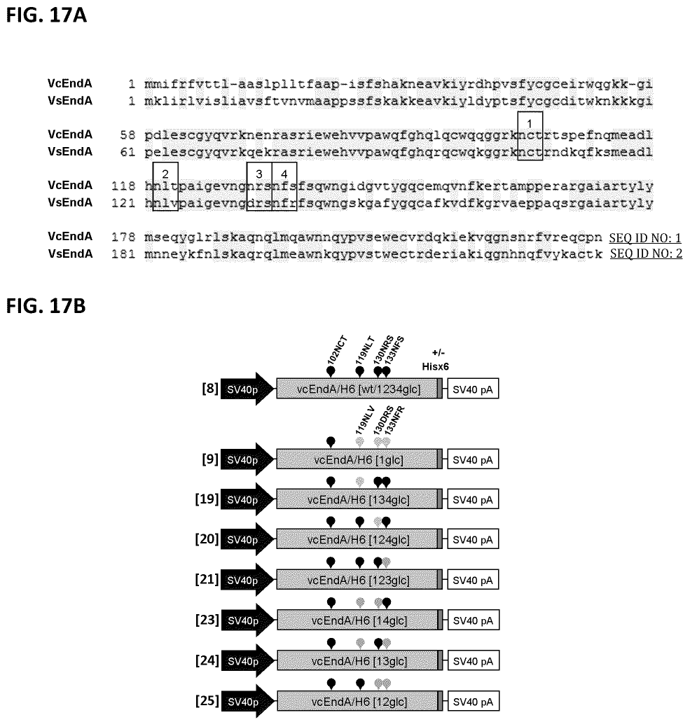

HIV-1 based vectors encoding GFP were produced from serum-free, suspension-adapted HEK293T-1.65S cells with different secreted nuclease encoding plasmids (0.5% or 5% total input pDNA) co-transfected with LV component pDNA. The secreted nucleases tested were all His-tagged at their C-termini for ease of detection by immunoblotting. All the sequon-mutated variants displayed in FIG. 17B were tested alongside wild type VcEndA (‘1234-glc’), SmNucA and VsEndA.

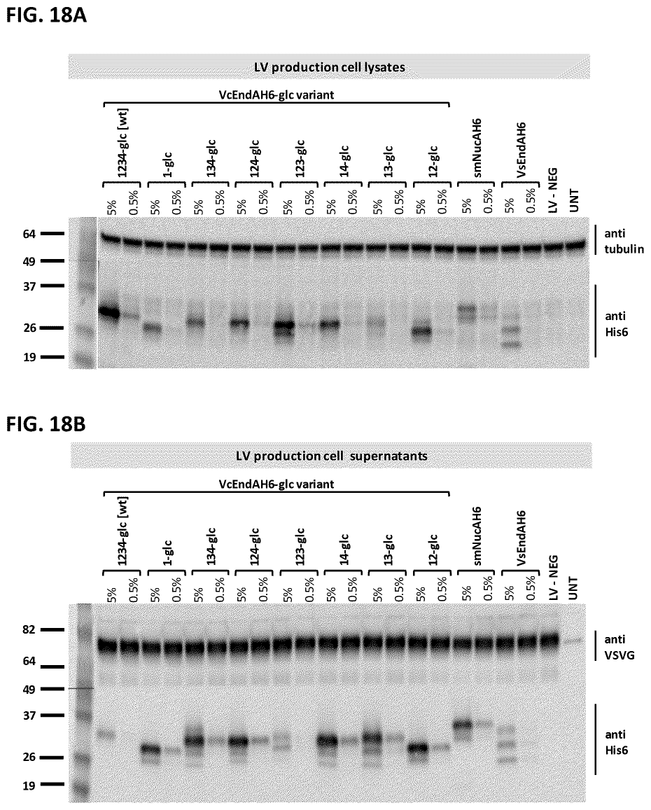

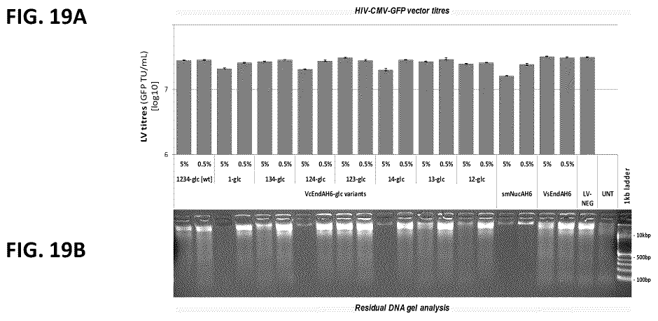

End-of-production cell lysates and concentrated supernatants were subjected to SDS-PAGE and Western blot, using anti-sera against His6-Tag (SEQ ID NO:27) (nucleases), tubulin (cell control) or VSVG (secretion control i.e. VSVG on LV virions)—see FIG. 18A-18B. These immunoblots demonstrated that the secreted nucleases (in all their glycosylated forms) were expressed in cells and secreted to some level during LV production. The wild type VcEndAH6-1234glc nuclease (and the VcEndAH6-123glc variant) were poorly secreted, demonstrating that the NxS/T sequon ablation approach can be used to generate novel, and improved secretion nuclease variants. To summarise, the 1glc and 12glc variants were predominantly singly glycosylated, whereas all the other forms of VcEndAH6 were doubly glycosylated. Through deduction, it can be concluded that if present, sequons at 102NCT (#1), 130NRS (#3) and 133NFS (#4) are utilized, except for VcEndAH6 (wt) and VcEndAH6-134glc, where predominantly only two of these sequons are actually utilized. LV titres were all above 1×10⁷ TU/mL, demonstrating that high titre vector can be produced in the presence of secreted nucleases (FIG. 19A).

Culture supernatants were also subjected to processing resulting in concentrated samples that were analysed for residual DNA content by visualization via gel electrophoresis (FIG. 19B). This analysis revealed a general correlation with residual DNA degradation and improved secretion. However, when the 130NRS sequon (#3) was present within VcEndAH6, DNA degradation was much less efficient, even if the nuclease was efficiently secreted; this indicates that the NRS sequon within VcEndA is preferentially utilised for N-glycosylation but that this modification results in attenuation of nuclease activity.

### Example 16: Degradation of Residual DNA within Lentiviral Vector Production Serum-Free, Suspension Cultures Using Secreted Nucleases at Different Set pH Conditions

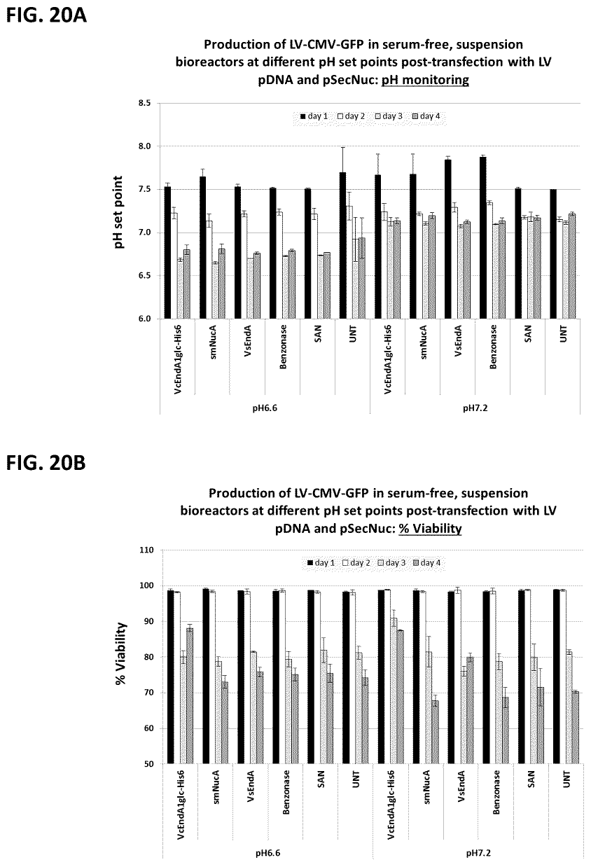

HIV-1 based vectors encoding GFP were produced from serum-free, suspension-adapted HEK293T-1.65S cells with different secreted nuclease encoding plasmids (5% total input pDNA) co-transfected with LV component pDNA, with cultures set at two different pH levels post-transfection; pH7.2 (slightly alkaline) and pH6.6 (slightly acidic)—see FIG. 20A-22B. Duplicate 12 mL cultures were set up using an AMBR-15 system, and pH levels (FIG. 20A) and cell viability (FIG. 20B) measured each day of the production run. On the day of transfection (day 2), pH set points were either 6.6±0.15 or 7.2±0.15 after the addition of pDNA/lipofectamine transfection mix, and these pH set points were maintained until LV harvest on day 4. The secreted nuclease plasmids used were pSV40-VcEndAH6-1glc (SEQ ID NO: 11, with C-terminal His6 (SEQ ID NO:27) Tag), pSV40-VsEndAH6 (SEQ ID NO: 2, with C-terminal His6 (SEQ ID NO:27) Tag), and pSV40-SmNucAH6 (SEQ ID NO: 3, with C-terminal His6 (SEQ ID NO:27) Tag). The remaining 12 transfections/cultures were spiked with 5% pBluescript and did not receive any secreted nuclease. These remaining 12 cultures were treated with either Benzonase® (four cultures), SAN (four cultures) or untreated (four cultures) for 1 hour prior to LV harvest; as per the pSecNuc transfected cultures (four in each case) these were paired into pH6.6 and pH7.2 set point test conditions.

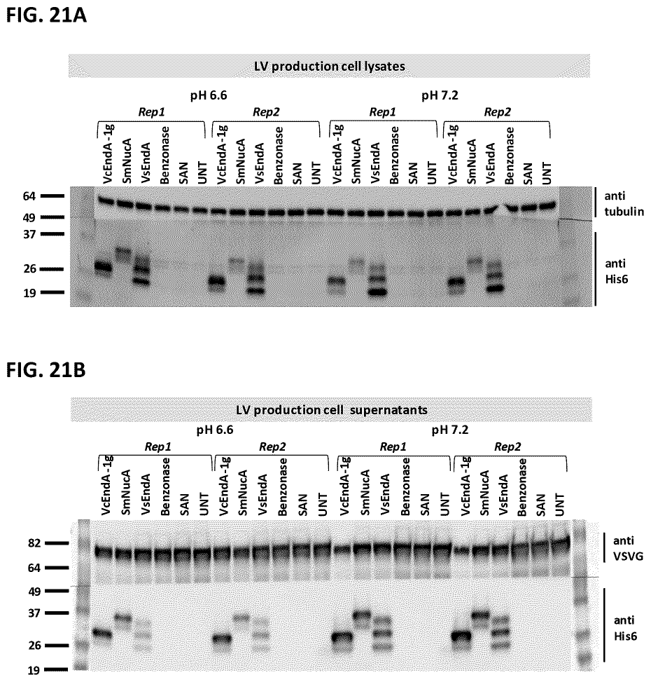

End-of-production cell lysates and concentrated supernatants were subjected to SDS-PAGE and Western blot, using anti-sera against His6-Tag (SEQ ID NO:27) (nucleases), tubulin (cell control) or VSVG (secretion control i.e. VSVG on LV virions)—see FIG. 21A-21B. These immunoblots demonstrated that the secreted nucleases (in all their glycosylated forms) were expressed in cells and efficiently secreted during LV production.

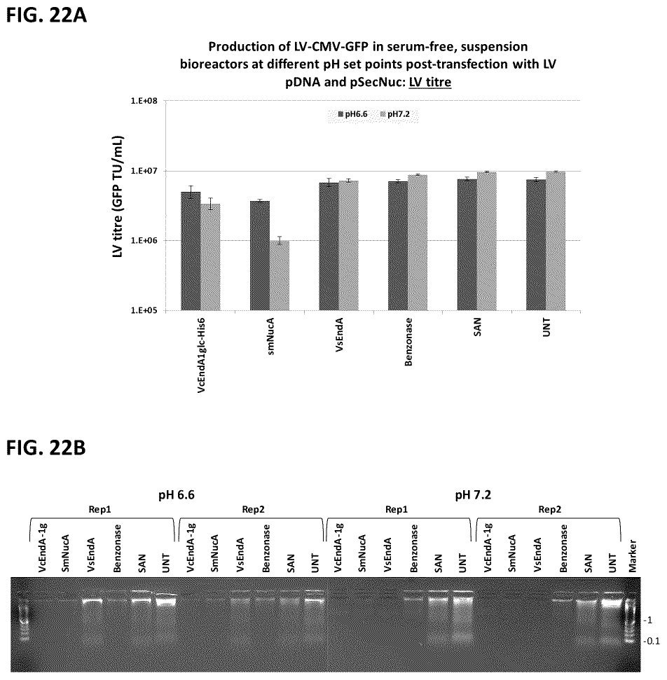

LV-CMV-GFP supernatants were titrated on HEK293T cells, which indicated that all conditions enabled LV production over 1×10⁶ TU/mL, with general similarity in production titres at both pH set points (FIG. 22A). Culture supernatants were also subjected to processing resulting in concentrated samples that were analysed for residual DNA content by visualization via gel electrophoresis (FIG. 22B). All cultures treated with secreted nuclease or Benzonase®/SAN displayed lower amounts of residual DNA compared to untreated, except that the secreted nucleases generally outperformed the commercial nucleases. VsEndAH6 displayed better residual DNA clearance properties at pH7.2 compared to pH6.6. However, both VcEndAH6-1glc and SmNucAH6 appeared to be efficient at clearing residual DNA at both pH set points; note that VcEndAH6-1glc achieved this whilst appearing to provide minimal impact on LV titres.

### Example 17: Degradation of Residual DNA within Lentiviral Vector Production Serum-Free, Suspension Cultures at Different Set pH Conditions: Comparing Activity of Two Different N-Glycan-Mutants of VcEndA

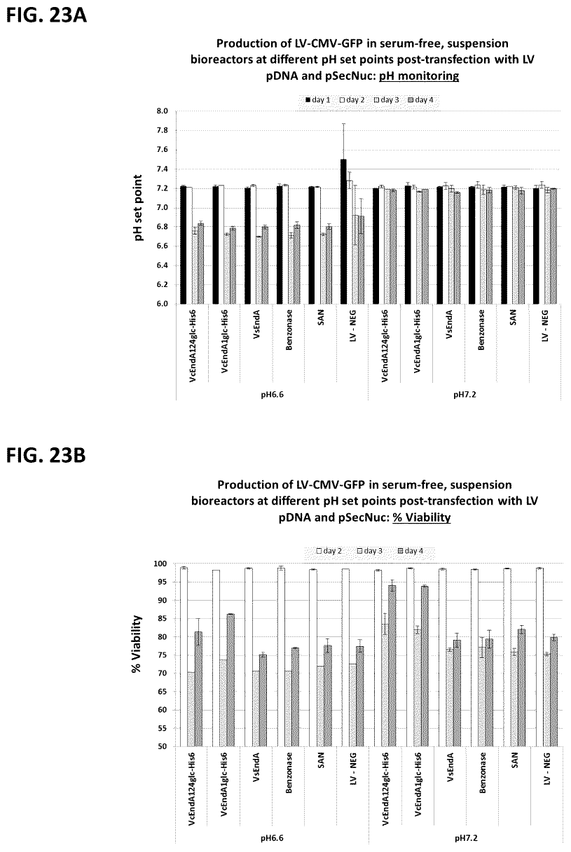

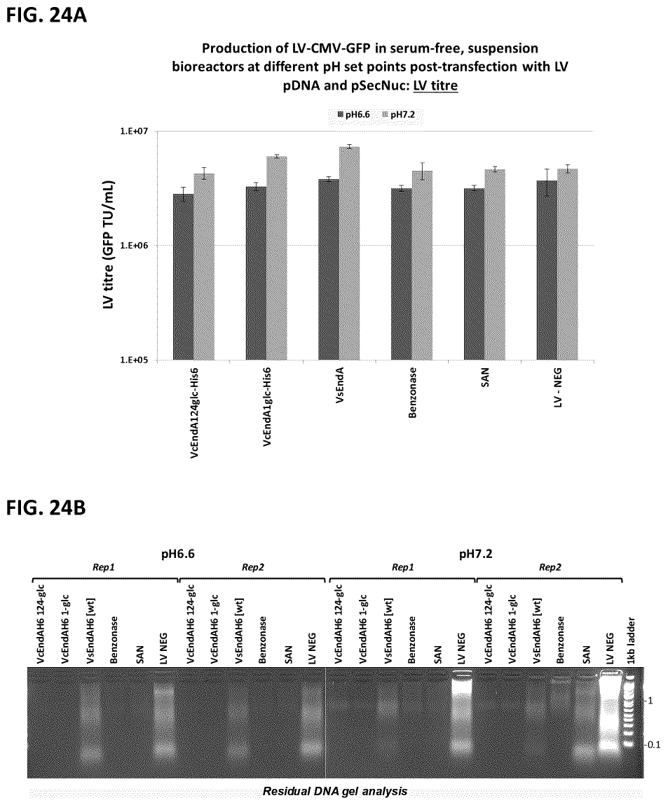

Example 15 identified three VcEndA variants with improved secretion and residual DNA clearance compared to wild type VcEndA, and had minimal/no impact on LV titres. These were VcEndAH6-1glc, -14glc and -124glc. Example 16 tested the VcEndAH6-1glc variant alongside VsEndAH6 and SmNucAH6 in stir tank mini-bioreactors under alkaline or acid culturing conditions, demonstrating that VsEndAH6 works less efficiently than VcEndAH6-1glc at acidic pH. Since VcEndAH6-1glc harbours mutated NxS/T sequons with altered residues based on VsEndA, it was unclear as to whether the improved activity of VcEndAH6-1glc under acidic conditions might be further improved if other NxS/T sequons were ‘re-instated’. A similar experiment as outlined in Example 16 was performed (production of LV-CMV-GFP vector and secreted nucleases in stir tank mini-bioreactors [AMBR-15] at pH6.6 or 7.2), and VcEndAH6-124glc, VcEndAH6-1glc and VsEndAH6 were compared against commercial nucleases (FIG. 23A-24B). Set points for pH and cell viability were recorded (FIG. 24A-24B), and LVs were titrated demonstrating that these nucleases had no impact on production titres (FIG. 24A). Culture supernatants were also subjected to processing resulting in concentrated samples that were analysed for residual DNA content by visualization via gel electrophoresis (FIG. 24B). This analysis revealed very similar levels of residual DNA clearance by VcEndAH6-1glc and VcEndAH6-124glc, with both variants out-performing VsEndAH6 as well as showing enhanced activity under acidic conditions. These data demonstrate that the VcEndAH6-1glc variant possess the full characteristics of activity at acidic pH despite the modifications introduced into its primary sequence, perhaps indicating that the altered residues do not contribute to nuclease activity that distinguishes VcEndA from VsEndA.

### Example 18: Use of Secreted and Cell-Retained Nucleases to Degrade Residual DNA During Production of AAV-Based Vectors

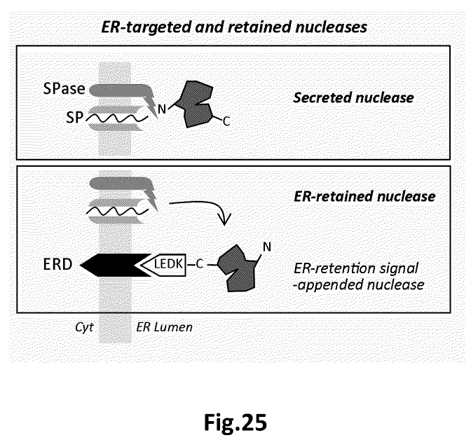

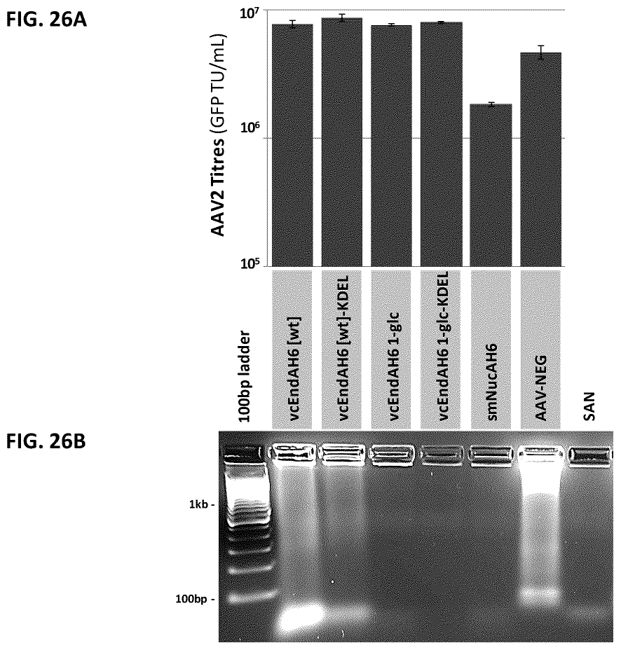

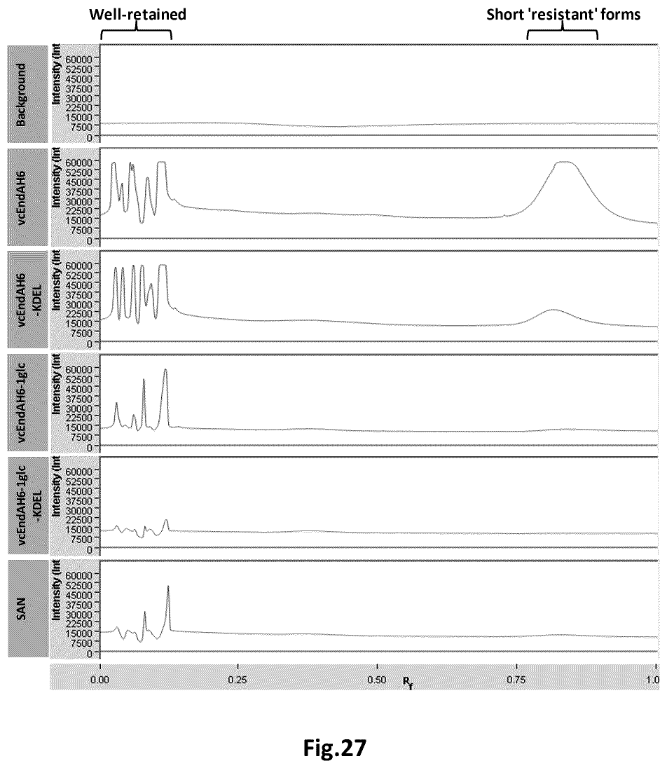

For production of viral vectors whereby the vector virions remain associated with production cells—such as AAVs and AdVs—it is anticipated that production cells may be lysed directly in situ within the bioreactor by addition of cell-disrupting agent such as detergent. In this case, secreted nuclease within the culture media will be present to degrade contaminating DNA. However, if production cells are partitioned away from the culture media (e.g. by precipitation or centrifugation or media change) then it is desirable for co-expressed nuclease to remain associated with cells in order to be present and active subsequently to cell lysis. This example shows how the C-terminal appendage of nuclease with the known ER-retention signal ‘KDEL’(SEQ ID NO: 21) improves residual DNA clearance from cell lysate during scAAV2-GFP production. FIG. 25 shows (in non-limiting terms) how an ER-retention signal can be appended to a nuclease secreted into the ER lumen resulting in its binding to an ‘ER retention-defective [ERD] complementation group’ protein receptor, such that is it continually cycled back to the ER lumen. To test this approach, VcEndAH6 [wt] (shown to be poorly secreted in HEK293T based cells; see FIG. 18A-18B) and VcEndAH6-1glc were engineered to contain a C-terminal ‘KDEL’(SEQ ID NO: 21) sequence. FIG. 26A-26B displays the results of scAAV2-GFP vector production by co-transfection of suspension, serum-free HEK293T cells with the indicated nuclease expression plasmids (5% of total input) together with AAV2 vector component plasmids (Genome:RepCap2:Helper ratio was 1:1:1). The SmNucAH6 nuclease was also tested. Two controls were included in which no nuclease expression plasmid was included (i.e. 5% pBluescript: ‘AAV-NEG’). After two days, cells were pelleted and separated from supernatant, and three freeze-thaw events were carried out in the presence of a detergent-based lysis buffer. Magnesium Chloride was added to all conditions (2 mM final), and SAN added to one of the controls to allow comparison to a commercial nuclease (effectively 75 U/0.5 mL cell pellet). Debris was cleared by low-speed centrifugation, and the resulting lysate filtered, before crude vector-containing supernatant was titrated on HEK293T cells (FIG. 26A) and residual DNA analysed by agarose gel electrophoresis (ethidium bromide) (FIG. 26B). This example demonstrates that high titre scAAV2-GFP can be produced in the presence of co-expressed nuclease, albeit that SmNucAH6 reduced titres by 3-4 fold at this level of plasmid input. The other nucleases tested had no impact on vector production at the 5% input level. Analysis of residual DNA clearance indicated that actually, a very good level of DNA digestion was achievable with VcEndAH6-1glc and smNucAH6 without the KDEL (SEQ ID NO: 21) sequence. The levels of these two secreted nuclease associated with cell lysates (see FIG. 18B) appear to be sufficient to allow efficient DNA clearance, and indicates that these are fully active proteins ‘on their way’ to being secreted from the cell. Nevertheless, the KDEL (SEQ ID NO: 21) appendage to VcEndAH6 [wt] and VcEndAH6-1glc appeared to allow improved DNA clearance compared to the non-KDEL (SEQ ID NO: 21) variants. As observed with lentiviral vector production, VcEndAH6-1glc was much more efficient than the VcEndAH6 [wt] version, and the VcEndAH6-1glc-KDEL (SEQ ID NO: 21) variant was capable of reducing residual DNA levels close to background levels when comparing densitometry profiles (FIG. 27) of the gel image in FIG. 26B. The VcEndAH6-1glc-KDEL (SEQ ID NO: 21) variant out-performed the other secreted nucleases tested as well as SAN in eliminating the small, ‘resistant’ forms of DNA typically observed in gel analysis. The example demonstrates the utility of secreted nucleases in clearing residual DNA in a cell-associated fashion but that this can be improved upon by the use of ER-retention signals.

### Example 19: Optimisation of Transfection Parameters to Generate Nuclease Helper Cells by Transient Transfection

One of the principal ways of applying the Secreted nuclease technology in a preferred embodiment will be to employ nuclease helper cells generated separately, and applied to the main viral vector production culture once viral vector particles are being produced. One approach is to transiently transfect cells—ideally the same base cell line that will be used for transient transfection by viral vector encoding plasmids—in parallel during the GMP manufacturing campaign. Conceivably, a GMP bank of transiently transfected nuclease helper cells could be generated (characterized and validated before freezing), which could then be revived and applied to the main viral vector production culture. The perceived advantage of the use of transiently transfected nuclease helper cells over stable, tet-regulated nuclease helper cells is the lack of requirement of a chemical inducer such as tetracycline or doxycycline. However the helper cell approach is applied (transient or stable), in a preferred embodiment an important parameter will be the total number of helper cells added to the main viral vector production culture in order to achieve a desirable (efficacious) level of secreted nuclease activity in the main viral vector production culture.

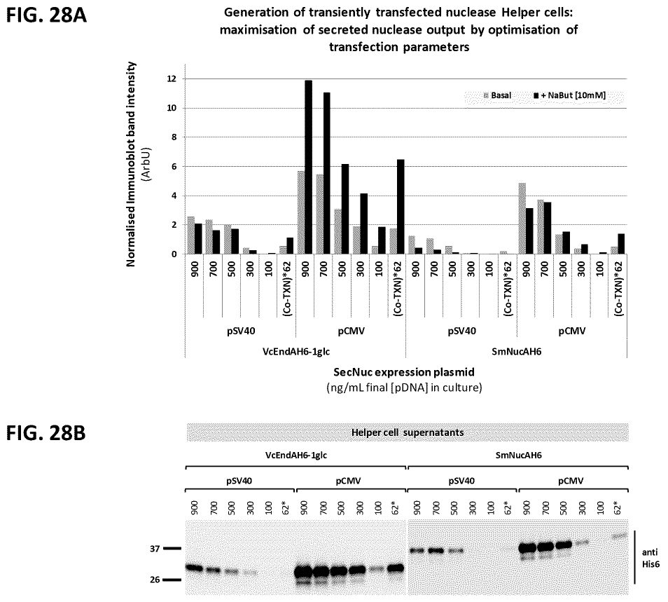

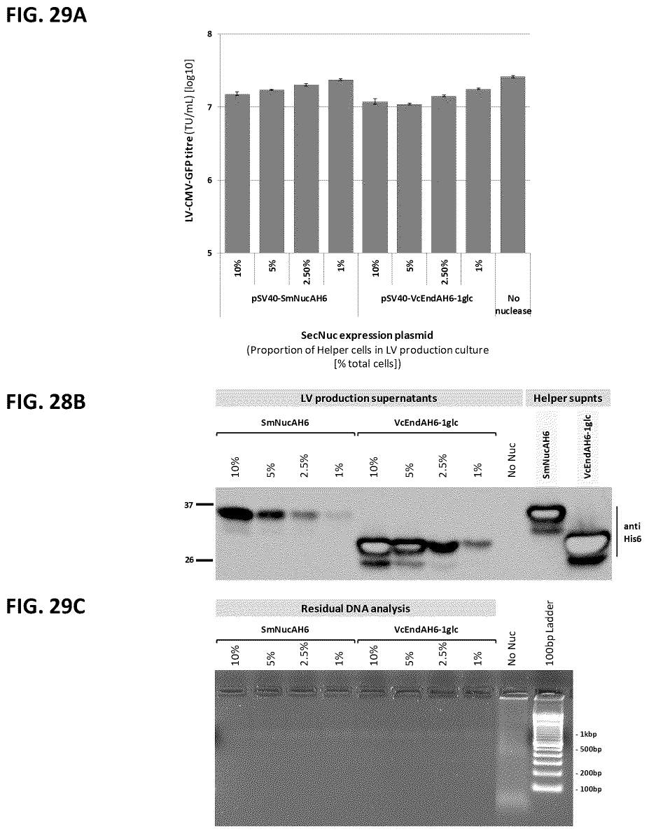

The approach of co-transfection of secreted nuclease plasmids together with viral vector plasmids (see previous Examples) has revealed a general range of percentage input levels (relative to the total amount of plasmid DNA being transfected) of 1-5%, depending on the strength of promoter being employed. However, in this approach the viral vector plasmids act as ‘carrier’ DNA for each other and also for the secreted nuclease plasmid. For a helper cell approach to be compatible with GMP production in a preferred embodiment only the secreted nuclease plasmid should be transfected into cells (without any ‘stuffer’ DNA), and moreover, it will be desirable to achieve maximal expression/secretion of the secreted nuclease from the helper cells in order to be able to apply low numbers of helper cells to the main viral vector production culture to achieve maximal clearance of residual DNA. To evaluate this, serum-free suspension HEK293T cells were transiently transfected with VcEndAH6-1glc or SmNucAH6 encoding plasmids driven by either the weaker SV40 promoter or stronger CMV promoter at a range of input amounts (100-to-900 ng per mL final culture). Transfections were carried out in two groups: with or without sodium butyrate induction (at 10 mM final concentration). The sodium butyrate induction was performed within 20 hours post-transfection (a typical regime for sodium butyrate induction of Lentiviral vector production) in order to mimic the effect of adding helper cells into the main viral vector production culture at (or beyond) the time of sodium butyrate induction (sodium butyrate is known to up-regulate expression of genes driven by promoters such as CMV in HEK293-based cells). A co-transfection control was included for each secreted nuclease plasmid, whereby pSecNuc (at 5% total; 62 ng per mL final culture) was mixed-in with pBlueScript (at 95% total); this was done to be able to compare secreted nuclease expression of helper cell cultures with the co-transfection approach exemplified in other Examples herein. Two days post-transfection, the supernatants from the transfected helper cells were harvested and analysed by immunoblotting to the secreted nucleases (anti-HisTag); cell lysates were also analysed by immunoblotting to the secreted nucleases and GAPDH. Secreted nuclease band intensity was quantified and normalized to GAPDH bands in order to assess the level of secreted nuclease expressed in cells (FIG. 28A), and this compared to immunoblots of secreted nuclease in the supernatants (FIG. 28B).

The data reveals that (as expected) choice of promoter strength correlates with expression levels of secreted nuclease in the cells, with the CMV-promoter driven cassettes achieving a higher level of expression than SV40-promoter counterparts. It was identified that transfection inputs of ≥700 ng plasmid DNA (per mL of cell culture, containing ≥8×10⁵ viable cells) achieved close-to-maximal levels of secreted nuclease expression. The effect of sodium butyrate induction was generally observed with the CMV-promoter driven cassettes; the boost to VcEndAH6-1glc expression was more obvious with VcEndAH6-1glc compared to SmNucAH6. This is in line with previous work indicating SmNucAH6 expression may be in some way ‘self-limiting’, building a further body of evidence that the VcEndAH6-1glc nuclease is a superior choice in application of the invention. When comparing the level of VcEndAH6-1glc expression achieved using pSV40-VcEndAH6-1glc (used mainly for the ‘co-transfection’ approach exemplified in other Examples herein) with the pCMV-VcEndAH6-1glc construct at ≥700 ng/mL input levels, the latter was able to achieve at least 10-fold more expression than the former.

### Example 20: Quantitation of Nuclease Activity Output of Nuclease Helper Cells when Spiked into Viral Vector Production Cultures

Example 19 demonstrated that nuclease helper cell cultures can be generated with extremely high levels of nuclease secretion and nuclease activity. Clearly this invention describes a number of ways of generating nuclease helper cells, and conceivably the nuclease output activity of a given helper cell culture may vary. Thus, in order to achieve desirable/efficacious levels of nuclease activity with viral vector production cultures by spiking-in helper cells, the number of helper cells being supplemented would vary depending on the nuclease activity output of the helper cells being employed. However, the most desirable approach will be one in which as few helper cells as possible are needed when adding to the main viral vector production culture so as to minimize impact on the vector production process (e.g. effects on viral vector production cells such as dilution effects and/or competition for growth metabolites). In order to achieve this in a preferred embodiment, maximal expression of the secreted nuclease will be desirable or, in some cases, necessary; this was exemplified in Example 19. To evaluate the efficiency of DNA clearance within viral vector production cultures by spiking-in helper cells, an initial experiment was performed whereby nuclease helper cells were generated by transient transfection of serum-free, suspension HEK293T cells with 750 ng plasmid DNA per mL culture, and were spiked into lentiviral vector production cultures at different percentage inputs relative to the total numbers of cells in the main lentiviral vector production vessel; these were 10%, 5%, 2.5% and 1%. In this initial experiment the SV40-promoter driven nuclease plasmids for VcEndAH6-1glc and SmNucAH6 were used. Cells from the main viral vector production and helper cultures were transfected with the relevant components in parallel, and then were counted ˜20 hours post-transfection before the appropriate volume of helper cell culture suspension (representing the above percentages) was added to replicate viral vector production cultures split-out from the original vector production culture. Normal vector production was then continued from this point (in this case, sodium butyrate induction occurred concomitantly with helper cell addition), and vector harvests taken for titration and analysis of nuclease secretion by immunoblot and for residual DNA content (FIG. 29A-29C). Vector titres produced with or without helper cells were >1×10⁷ TU/mL, and were minimally impacted at 10% and 5% helper cell inputs (<2-fold), perhaps indicating that addition of greater than 10% of helper cells into viral vector production cultures may not be desirable (FIG. 29A). The levels of secreted nuclease achieved in the helper cell cultures were extremely high (as denoted by burn-out of bands on the immunoblots), and the resulting levels of secreted nuclease in the vector production cultures correlated well with the input percentages (FIG. 29B). Again, a greater level of VcEndAH6-1glc protein level was achieved compared to SmNucAH6. Surprisingly, extremely good levels of residual DNA clearance were observed for all secreted nuclease conditions, even at the lowest input level of 1% (FIG. 29C). This indicated that the nuclease helper cell approach can be used using secreted nuclease expression cassettes using promoters of modest activity but importantly in a preferred embodiment, if a stronger promoter is used then even fewer nuclease helper cells may be added to the viral vector production culture, potentially avoiding any observable impact on vector titres.

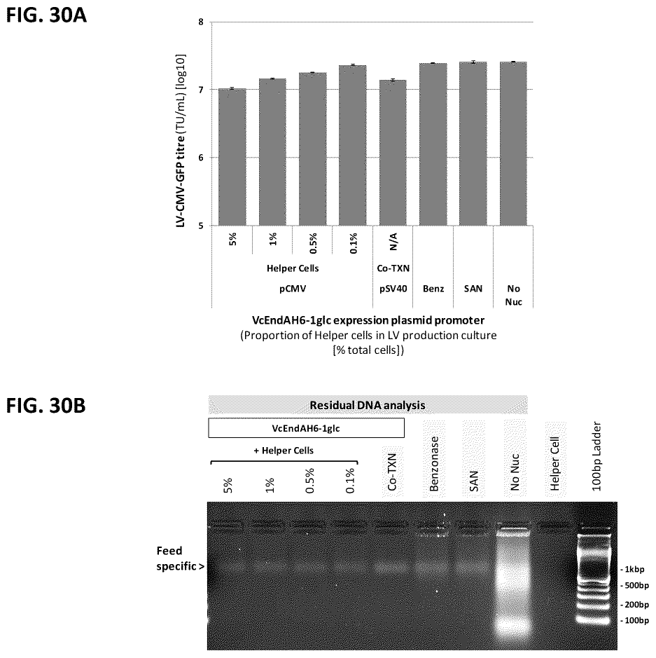

A further evaluation of the nuclease helper cell approach was undertaken by focusing on the use of the pCMV-VcEndAH6-1glc plasmid, and reducing the number of helper cells added to the main viral vector production culture. helper cell and lentiviral vector production cells were transfected with the respective plasmids in parallel, and then different proportions of the helper cells were spiked into the vector production cultures ˜20 hours post-transfection at the sodium butyrate induction point. Similarly as before, nuclease helper cells were generated by transient transfection of serum-free, suspension HEK293T cells with 750 ng plasmid DNA per mL culture. Since it was previously demonstrated that the CMV-promoter driven cassette yielded >10-fold more VcEndAH6-1glc protein than the SV40-promoter, the range of cell input of the helper cells was taken down as far as just 0.1% (relative to the number of cells within the vector production culture). After considering viable cell counting, this equated to 30 μL of helper cell culture added to 40 mL of vector production culture. In this experiment additional controls included standard Benzonase/SAN (5 U/mL) treatment 1 hr prior to harvest, and the 5% ‘co-transfection’ control (pSV40-VcEndAH6-1glc mixed with LV components) was included to directly compare the helper cell approach with the ‘co-transfection’ approach. As before, vector production continued as normal, and vector harvests were taken for titration and analysis of residual DNA content (FIG. 30A-30B). Vector titres produced with or without helper cells were >1×10⁷ TU/mL, and were minimally impacted at 5% and 1% helper cell inputs (<3-fold) (FIG. 30A).

The assessment of residual DNA clearance in concentrated (44-fold) vector production cultures revealed extremely good clearance of residual DNA by secreted VcEndAH6-1glc, which appeared to be the same or better than that of the Benzonase/SAN treated vector samples (FIG. 30B). Remarkably, when just 0.1% of the total number of cells within the vector production culture comprised helper cells, the level of apparent residual DNA clearance was just as effective compared to the high input levels of helper cells. Interestingly, a ‘nuclease-resistant’ band (‘Feed-specific’) was observed for all concentrated vector supernatants but not in the concentrated helper cell supernatant. This indicated that this band was specific to lentiviral vector-containing supernatant, suggesting that it could be (cellular) RNA—possibly residing within virions—protected from the RNAse activity of VcEndAH6-1glc. Looking back through many of the other Examples of use of secreted nucleases during lentiviral vector production, a similar band can sometimes be observed. In this Example, the fold-concentration of vector supernatants was greater than previous Examples, perhaps increasing the sensitivity of detection of this band in this experiment. However, such a band is not observed in purified vector material (See Examples 11 and 21), indicating that this material unlikely to be within virions and can be removed by further processing such as buffer-exchange. Given the extremely efficient removal of all apparent Ethidium bromide stained contaminants by VcEndAH6-1glc in 5 L bioreactor production scales demonstrated in Example 21, it seems likely that the occasional appearance of this band in agarose gel analysis could be due to some variable and unidentified contaminant—possibly free cellular/ribosomal RNA—which is generally not detected within downstream-processed vector.

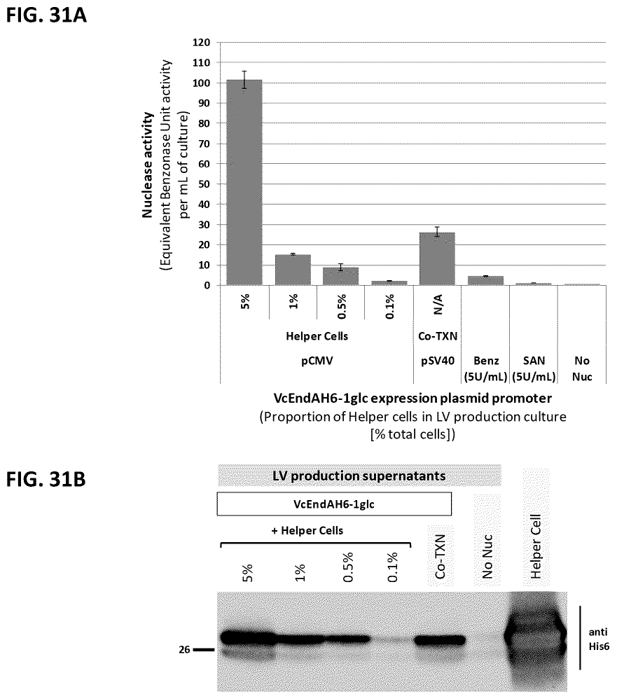

To be able to link the level of secreted nuclease achieved in both helper cell and viral vector production cell cultures to unit-defined nuclease activity, the culture supernatants were evaluated for nuclease activity by DNAse Alert assay, and also by immunoblot to VcEndAH6-1glc (anti-HisTag). A commercial Benzonase® enzymes stock of known nuclease activity was used as standard curves within the assay. FIG. 31A-31B displays this relationship, and enables this invention to describe desirable secreted nuclease expression ranges in terms of objective nuclease activity units. The nuclease activity (FIG. 31A) and immunoblot (FIG. 31B) data correlated very well, not only between both assay types but also for the proportion of nuclease helper cells spiked into the vector production cultures. Importantly, the DNAse Alert assay was validated as a highly accurate method because the control vector cultures treated with 5 U/mL Benzonase® were calculated to contain an average of 4.5 U/mL. The data show that for vector production cultures comprising 5% nuclease helper cells the activity of nuclease within the culture media was ˜100 Benzonase® unit equivalents per mL. The nuclease activity of the cultures comprising 1%, 0.5% and 0.1% nuclease helper cells were 15.2, 8.8 and 2.1 Benzonase® unit equivalents per mL, in good agreement with the proportional reduction in helper cells added to the vector production culture. In this experiment, the ‘co-transfected’ vector production culture achieved 26.3 Benzonase® unit equivalents per mL. Importantly, whilst the 0.1% nuclease helper cell-containing vector production culture ‘only’ achieved 2.1 Benzonase® unit equivalents per mL compared to the ‘standard’ vector cultures treated with 5 U/mL of Benzonase® or SAN, the clearance of residual DNA was at least as good as these standard treatments. This indicates that the use of secreted nuclease holds advantage over use of commercial nucleases not only from the point of view of achieving higher levels of nuclease activity (at much reduced cost) but also that supplying a tonic level of nuclease through-out vector production (even at lower levels) provides a more efficacious mode of residual DNA clearance. Using the nuclease activities measured within the vector production cultures, it is possible to back-calculate the amount of secreted nuclease activity within the helper culture; this was ˜2000 Benzonase® unit equivalents per mL.

### Example 21: Use of VcEndAH6-1glc to Degrade Residual DNA with Serum-Free, Suspension Bioreactors to a Level that Negates the Use of Further Nuclease Treatment in the Downstream Process

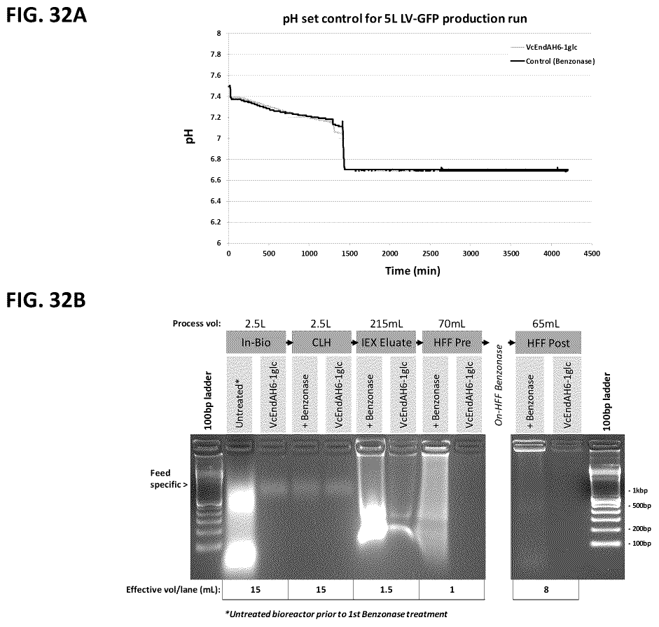

Examples 10 and 11 show that the application of secreted nuclease to lentiviral vector production cultures in mid-to-large scale suspension bioreactors, aids in the clearance of residual DNA. The use of VsEndAH6 at 5 L scale suggested that this specific nuclease may be limited in activity in cultures at lower pH because maximal clearance of residual DNA in downstream process material was still aided by the use of commercial nuclease added on the hollow fibre cartridge. This pH sensitivity was later verified and led to the development of the ‘pH-tolerant’ VcEndAH6-1glc nuclease (Examples 15-17). To assess if the VcEndAH6-1glc nuclease applied in the upstream phase could enable the retraction of the commercial nuclease treatment during the same downstream process (during dia-/ultra-filtration), lentiviral vector encoding GFP was produced in the following manner. Suspension, serum-free adapted HEK293T cells were seeded into two 5 L bioreactors transfected with HIV-1 based lentiviral vector components (GFP expressing genome) together with either pBluescript (control; for standard Benzonase® treatment) or pSV40-VcEndAH6-1glc at 5% total plasmid input. Twenty hours post-transfection, sodium butyrate was added to all bioreactors at a final concentration of 10 mM. One hour prior to vector harvest, ‘In Bio’ samples were taken (cells removed and supernatant filtered), and then the control bioreactor was inoculated with Benzonase® at 5 U/ml final concentration together with 2 mM MgCl₂. The pSV40-VcEndAH6-1glc co-transfected bioreactor received only 2 mM MgCl₂. Both bioreactors were incubated for 1 hour under standard growth conditions prior to harvest, where upon 5 L of the culture was clarified (10 μm>0.45 μm) and samples taken (CLH). Approximately 2.5 L of the harvests were then subject to downstream processing. Material was subjected to ion-exchange (IEX) chromatography, generating ˜215 mL IEX Eluate, before further processing by dia-/ultra-filtration using hollow fibre cartridges reduced this volume to 65-70 mL. The first step allowed buffer exchange out of salt ‘HFF-Pre’ (Pre-Benzonase® treatment)—where samples were taken—and then 400 U/ml Benzonase® was added to both the standard (‘+Benzonase®’) processed vector and the VcEndAH6-1lgc treated vector; this was done to assess any further benefit of an on-HFF nuclease step, should the secreted-nuclease vector material require it. Finally, buffer exchange occurred to remove Benzonase® (‘HFF Post’). FIG. 32A shows the pH profiles of the two bioreactors during the production run, demonstrating that the target pH of 6.7 was achieved. The clarified harvest vector (CLH) samples were titrated by FACS assay and revealed titres of 1.7×10⁷ and 1.0×10⁷ TU/mL for standard and VcEndAH6-1glc vectors, respectively. Samples from the whole production process were concentrated by centrifugation on 3K cut-off centrifugal devices before samples were loaded onto agarose gels (Ethidium bromide) for residual DNA analysis (FIG. 32B). The residual DNA analysis demonstrates excellent clearance of residual DNA using VcEndAH6-1glc, and that the use of this nuclease in upstream production essentially allows removal of the vast majority of contaminating residual DNA at the dia-/ultra-filtration step—no further use of commercial nuclease is required. To expand, whilst there is detectable residual DNA within the VcEndAH6-1glc vector eluate from the IEX column, this is apparently in a form that could be extracted from the vector material during buffer exchange. Given that the dia-/ultra-filtration hollow fibre pore size was 0.2 μm, this indicated that the residual DNA that co-eluted with the vector was smaller (or was within smaller protein complexes) than the vector particles. Note also the presence of the ‘Feed-specific’ band (see Example 20) in unprocessed samples, which is certainly lost at or before the dia-/ultra-filtration step. Residual DNA could still be observed within the ‘HFF post’ control (2× treated Benzonase®) vector material. This represents for the first time the ability to remove residual DNA from lentiviral vector production using a nuclease approach that is not dependent on use of commercial, recombinant nuclease.

### Example 22: The Use of the KDEL (SEQ ID NO: 21) Retention Signal to Enrich VcEndAH6-1glc-KDEL (SEQ ID NO: 21) in Cell Fractions

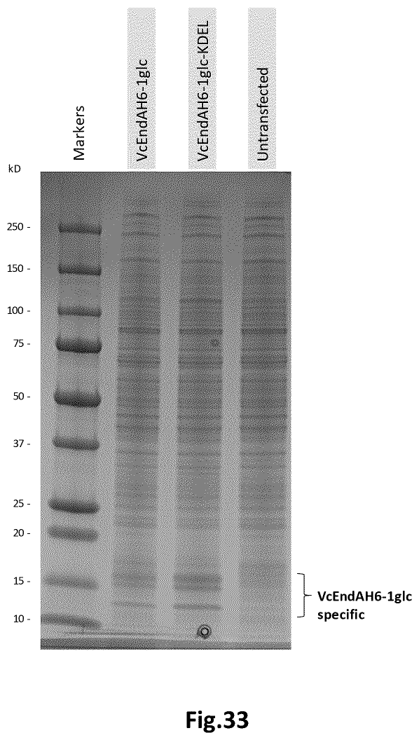

Example 18 demonstrated the use of secreted and ER-retained nucleases to clear residual DNA during upstream production of AAV vectors. FIG. 33 displays SDS-PAGE analysis of harvest cell lysates resulting from transfection of serum-free, suspension HEK293T cells with either pSV40-VcEndAH6-1glc or pSV40-VcEndAH6-1glc-KDEL (SEQ ID NO: 21). The analysis supports the previous work indicating that nuclease expression can be directed such that the nuclease is retained/enriched within cells by use of the ER-retention signal appended at the C-terminus of the nuclease protein.

### Materials and Methods

### Nuclease Expression Constructs

Nuclease open-reading frames were codon and sequence optimized for high expression in Homo sapiens by GeneArt. smNucA (NCBI Reference Sequence: WP_047571650.1), VsEndA (GenBank: CAQ78235.1), VcEndA (NCBI Reference Sequence: WP_000972597.1), BacNucB (NCBI Reference Sequence: WP_003182220.1). All nucleases were evaluated by designing gene expression cassettes that used the SV40 promoter and SV40 polyadenylation signal (FIG. 3A-3C). Typically, entire expression cassettes were synthesized as one fragment encoding the promoter-5′utr-nuclease-3′utr-polyA and received within GeneArt's pMK backbone. The 3′utr regions were designed to harbor an alternative C-terminus fused to a 6×His-tag (SEQ ID NO: 27), such that His-tagged variants of each nuclease could be generated by simple restriction enzyme digestion and re-ligation of the plasmid. Construction of ER signal peptide variants and NxS/T sequon deletion/insertion variants were typically carried out by cloning of re-derived, synthetic fragments (GeneArt) into existing nuclease plasmids described above. Inducible nuclease expression cassettes (FIG. 3D) were cloned by inserting the nuclease ORF into a re-derived version pf pcDNA5/TO via HindIII/NotI using standard cloning techniques. Broadly, digestions were carried out at 37′C for 1-2 hours with enzyme (5-10 units per microgram of pDNA), followed by a 10 minutes ‘Rapid’ dephosphorylation reaction for backbone fragments (Roche) and then run on a 1% agarose gel for 1 hour at 100V. The relevant bands were extracted using the QIAQuick gel extraction kit (QIAGEN). Ligations were carried out with backbone:insert ratios of 1:3 using the ‘Rapid’ DNA ligation kit (Roche) in 20 μL volumes under recommended conditions. Approximately 25-50 μL Turbo (NEB) or Stbl2 (Thermo) competent cells were transformed with 2-3 μL of ligation reactions. Bacteria were grown on LBKAN agarose plates, and in LBKAN media for minipreps. After restriction enzyme analysis and sequence confirmation plasmid DNA was prepared by Plus Mega kit (QIAGEN).

### Adherent Cell Culture, Transfection and Lentiviral Vector Production

HEK293T cells were used for vector production and titration. The cells were maintained in complete media (Dulbecco's Modified Eagle Medium (DMEM) (Sigma) supplemented with 10% heat-inactivated (FBS)(Gibco), 2 mM L-glutamine (Sigma) and 1% Non-essential amino acids (NEAA) (Sigma)), at 37° C. in 5% CO².

The standard scale production of HIV-1 vectors in adherent mode was in 10 cm dishes under the following conditions (all conditions were scaled by area when performed in other formats): HEK293T cells were seeded at 3.5×10⁵ cell per ml in complete media and approximately 24 hrs later the cells were transfected using the following mass ratios of plasmids per 10 cm plate: 4.5 μg Genome, 1.5 μg Gag-Pol, 1.1 μg Rev, 0.7 μg VSV-G (i.e. total of 7.8 μg/plate). Where appropriate, nuclease expression plasmid was spiked into this vector mix at the indicated % input of total pDNA (typical range from 0.001 to 10%).

Transfection was mediated by mixing DNA with Lipofectamine 2000CD in Opti-MEM as per manufacturer's protocol (Life Technologies). Sodium butyrate (Sigma) was added ˜18 hrs later to 10 mM final concentration for 5-6 h, before 10 ml fresh serum-free media replaced the transfection media. Typically, vector supernatant was harvested 20-24 h later, and then filtered (0.22 μm) and frozen at −20/−80° C. As a positive control for nuclease treatment, typically Benzonase® was added to the harvests at 5 U/mL for 1 hour prior to filtration.

HEK293T.GFP.PrCL (stable cell line) was developed in-house using the established HEK293T.tetR14 adherent cell line. All the packaging components and the HIV-GFP genome were previously stably transfected into HEK293T.TetR cells along with selection markers. Rev and VSV-G were placed under the selection of Zeocin (Zeo), Gag-Pol was under the selection of Blasticidin (Bsr) and the GFP genome was under the selection of Hygromycin (Hyg). For evaluation of the secreted nuclease approach within HIV-1 based vector producer cell lines, nuclease-expression plasmids were transfected into cultures at the indicated % input of total (where pBlueScript was added as stuff to a total of 7.8 μg/10 cm plate or equivalent scaled amount/area). To induce vector production, doxycycline was added from the Sodium butyrate induction step at a final concentration of 1 μg/mL.

### Suspension Cell Culture, Transfection and Lentiviral Vector Production

HEK293T.TetR14S (stably integrated codon-optimised tetR) and HEK293T.1-65s suspension cells were grown in Freestyle+0.1% CLC (Gibco) at 37° C. in 5% CO², in a shaking incubator (25 mm orbit set at 190 RPM). All vector production using suspension was carried out in a 125 ml shake flasks at a working volume of 25 ml or in AMBR-15 bioreactors at a working volume of 12 mL. HEK293 Ts cells were seeded at 8×10¹ cells per ml in serum-free media and were incubated at 37° C. in 5% CO², shaking, through-out vector production. For AMBR-15 bioreactor production, the cultures were set to pH7.2. Approximately 24 hrs after seeding the cells were transfected with a mix of vector component plasmids encoding genome (GFP), gagpol, rev and VSVG. Where appropriate, nuclease expression plasmid was spiked into this vector mix at the indicated % input of total pDNA (typical range from 0.1 to 10%).

Transfection was mediated by mixing DNA with Lipofectamine 2000CD in Opti-MEM as per manufacturer's protocol (Life Technologies). For AMBR-15 bioreactor production, the cultures were set to pH6.6 or pH7.2 immediately post-transfection. Sodium butyrate (Sigma) was added ˜18 hrs later to 10 mM final concentration. Typically, vector supernatant was harvested 20-24 h later, and then filtered (0.22 μm) and frozen at −20/−80° C. As a positive control for nuclease treatment, typically Benzonase® or SAN was added to the harvests at 5 U/mL for 1 hour prior to filtration. For AMBR-15 bioreactor production, pH and cell viability was monitored through-out the process.

### Production of Lentiviral Vectors in 0.5 or 5 L Stir-Tank Bioreactors and Downstream Processing

The suspension adapted HEK293T cell line, clone 1.65 s and supplemented serum-free media were used for the bioreactors studies. Cells cultured from the MCB were revived and scaled-up in Erlenmeyer flasks, and incubated in a pre-equilibrated 5% CO₂ in air incubator at 37° C. with a humidified atmosphere. The cells were kept in suspension on an orbital shaker platform.

Bioreactors were actively managed throughout for dissolved oxygen and pH; metabolite and by-product levels were also monitored. Cell viability was monitored, and antifoam was used if appropriate. Bioreactor cultures were co-transfected with four plasmids; pHIV-CMV-GFP (genome), pSynGPK (gagpol), pOB-Rev and pOB-VSVG, using a transfection reagent. Where indicated, pNuclease was typically spiked into pDNA mixes at 5% of total. The bioreactor cultures were induced 20 hours post transfection with 10 mM of sodium butyrate. Vector harvests were taken 24 hours post-induction, with optional treatment with 5 U/mL or 25 U/mL commercial nucleases (Benzonase® or SAN-HQ) for 1 hour prior to harvest were indicated.

Vector supernatants were pre-clarified (≥10 μm) and then further clarified by 0.22-0.45 μm filtration.

At least 1 L clarified vector harvests were processed by ion-exchange chromatography using Sarto-Q cartridges, typically including low concentration salt wash and pre-elution wash buffers, before elution in >1M NaCl-containing buffer and immediate dilution of the eluate to preserve vector virion activity. Eluate was then quickly subjected to Hollow Fibre dia/ultrafiltration to first buffer exchange into low salt conditions, before 400 U/mL commercial nuclease and 2 mM magnesium chloride was added as a second nuclease treatment polishing step. Finally, a final buffer exchange was performed to remove the commercial nuclease.

### Generation of Nuclease Helper Cells by Transient Transfection Achieving Maximal Nuclease Expression

The suspension adapted, serum-free HEK293T cells were seeded at 8×10⁵ cell per mL in parallel to the main viral vector production cultures and grown overnight in Freestyle+0.1% CLC (Gibco) at 37° C. in 5% CO₂. The next day cells were transfected with >750 ng pCMV-VcEndAH6-1glc per mL of culture using Lipofectamine 2000CD (vector production cultures were transfected with vector components). At the point of sodium butyrate induction (10 mM final), nuclease helper cells were inoculated at the target cell input levels to achieve the stated percentage of total cell count in the main vector production vessel. Vector production then continued as described above.

### Lentiviral Vector Titration Assays

For lentiviral vector titration, HEK293T cells were seeded at 1.2×10⁴ cells/well in 96-well plates. GFP-encoding viral vectors were used to transduce the cells in complete media containing 8 mg/ml polybrene and 1×Penicillin Streptomycin for approximately 5-6 hrs after which fresh media was added. The transduced cells were incubated for 2 days at 37° C. in 5% CO². Cultures were then prepared for flow cytometry by FACSVerse (BD Biosciences) percent GFP expression was measured and vector titres were estimated using predicted cell count following the 2-day incubation.

### AAV Vector Production

For AAV vector production, HEK293T-1.65S serum-free, suspension adapted cells were seeded into culture vessels at 8×10⁵ cells/mL ˜20 hours prior to transfection. At transfection, self-complementary (sc) AAV-GFP vector production was initiated by transfecting cells with equal masses of pscAAV-CMV-GFP (genome), pRepCap2 (encoding all necessary viral packaging components, including assembly-activating protein [AAP]) and pHelper (Adeno E2A, E4 and VA RNA functions), together with either nuclease encoding plasmids or pBluescript (negative control) at 5% mass of total pDNA. Transfection was mediated by mixing DNA with Lipofectamine 2000CD in serum-free media as per manufacturer's protocol (Life Technologies). After two days, cells were harvested by centrifugation at low speed and cells lysed by three freeze-thaw cycles in the presence of detergent lysis buffer. Next, the lysate was pipetted up-and-down in 8 cell pellet volumes of serum-free DMEM. Magnesium Chloride was added to lysate at a final concentration of 2 mM, and incubated for 1 hour at 37° C. For the ‘commercial’ positive control, salt active nuclease (SAN-HQ; Articzymes) was added at 25 U per 0.5 mL effective cell pellet volume of a replicate Negative control lysate and incubated at 1 hour at 37° C. in the presence of 2 mM MgCl₂. Negative control lysate was incubated without nuclease. Treated cell lysates were centrifuged at low speed to remove debris, and then supernatants filtered (0.45 μm) prior to storage at −20° C.≤prior to further analysis.

### AAV Vector Titration Assays

For AAV vector titration, HEK293T cells were seeded at 1.2×10⁴ cells/well in 96-well plates. GFP-encoding viral vectors were used to transduce the cells in serum-free media containing 1×Penicillin Streptomycin for approximately 5 hrs after which fresh, serum-containing media was added. The transduced cells were incubated for 3 days at 37° C. in 5% CO². Cultures were then prepared for flow cytometry by Attune NxT (Thermofisher); percent GFP expression was measured and vector titres were estimated using cell counts and vector dilutions.

### Residual DNA Detection by PicoGreen® Assay

Quant-iT™ PicoGreen® dsDNA Assay Kit (Life Technologies) was used to assay the residual DNA in harvested vector material. Lambda stock DNA was serially diluted in Tris and EDTA (TE) buffer to prepare a DNA standard. TE buffer was also used to dilute harvested vector samples 1:10 before adding the PicoGreen assay reagent (prepared as per the manufacturer's protocol) and incubating the samples, in the dark, for 2 to 5 mins. The fluorescence of the PicoGreen reagent, in the samples, was measured with top read using an EM Microplate Reader (Gemini).

### Residual DNA Detection by Quantitative PCR Assay

DNA was extracted from vector samples using a QIAamp DNA Mini Kit (QIAGEN). During the extraction process, mock samples, containing nuclease free water (NFW) (Gibco) were used as in process controls (IPCs).

Extracted DNA was subjected to 18S quantitative Polymerase Chain Reaction (PCR) (qPCR); the reaction amplified a short fragment of cell derived 18S DNA. The mix included SYBR® Green PCR Mastermix (Life Technologies), the 18S primers (prepared in 10 mM Tris-HCl, pH7) (Sigma) and NFW.

(SEQ ID NO: 22)
The Forward primer: 5′ACCGCAGCTAGGAATAATGG 3′
(SEQ ID NO: 23)
The Reverse primer: 5′CTCAGTTCCGAAAACCAACA 3′

To generate a DNA standard, linearised 18S plasmid was serially diluted from 1×10⁶ copies/5 μl to 1×10² copies/5 μl. Samples were diluted 1:5 before the reaction mix was added. The reaction was carried out under standard chemistry qPCR conditions using QuantStudio™ 6 Flex System (Life Technologies). The qPCR run was set up and analysed the using QuantStudio™ 6 Flex System Software (Life Technologies).

The controls set up include a no template control (NTC), to control for PRC reaction mix contamination, a IPC sample, to control for DNA contamination during the extraction process and finally, to monitor PCR inhibition, a spiked PCR inhibition control (SPIC) was included by spiking 1×104 copies of 18S standard DNA into the IPC sample.

Extracted DNA was subjected to KanR quantitative Polymerase Chain Reaction (PCR) (qPCR); the reaction amplified a short fragment of plasmid derived KanR DNA. The mix included TaqMan® Universal PCR Master Mix (Life Technologies), the KanR primer and probe set (prepared in 10 mM Tris-HCl, pH7) (Sigma) and NFW.

(SEQ ID NO: 24)
The Forward primer: 5′ AGATGGATTGCACGCAGGTT 3′
(SEQ ID NO: 25)
The Reverse primer: 5′ TGCCCAGTCATAGCCGAATAG 3′
(SEQ ID NO: 26)
Probe: 5′ (FAM) CTCCACCCAAGCGGCCGGA (TAMRA) 3′

To generate a DNA standard, linearised KanR+ plasmid was serially diluted from 1×10⁶ copies/5 μl to 1×10² copies/5 μl. Samples were diluted 1:5 before the reaction mix was added. The reaction was carried out under standard chemistry qPCR conditions using QuantStudio™ 6 Flex System (Life Technologies). The qPCR run was set up and analysed the using QuantStudio™ 6 Flex System Software (Life Technologies).

The controls set up include a no template control (NTC), to control for PRC reaction mix contamination, a IPC sample, to control for DNA contamination during the extraction process and finally, to monitor PCR inhibition, a spiked PCR inhibition control (SPIC) was included by spiking 1×104 copies of KanR standard DNA into the IPC sample.

### Visualisation of Residual DNA Detection by Gel Electrophoresis

LV crude vector supernatants were filter-clarified and optionally treated with Proteinase-K at 37° C. for 1 hour. Clarified samples were centrifuged in Amicon Ultra-15 3K cut-off filter units to concentrated DNA by up to 150-fold. Samples were subjected to standard electrophoresis on 2% agarose/TBE gels (DNA visualised by Ethidium bromide) and 1 kbp/100 bp ladders run in parallel.

### Quantification of Nuclease Activity by DNAse Alert™ Assay (Referred to Herein as “Assay 1”)

The DNAse Alert™ assay (IDT) can be, and was, used to quantify nuclease activity within viral vector culture media. The basis of the assay is the use of fluorescently labelled and quenched nucleotide probes that emit a fluorescence signal only when degraded by a nuclease. The kit was used under manufacturers recommendations, using a standard curve composed of serially diluted Benzonase® from 5 U/mL to 0.08 U/mL. The viral vector culture supernatant containing secreted nuclease should be serially diluted such that the fluorescence activity falls within the upper and lower limits of the standard curve. The unit definition of Benzonase® (at the time of this work) is: “One unit will digest sonicated salmon sperm DNA to acid-soluble oligonucleotides equivalent to a ΔA260 of 1.0 in 30 min at pH 8.0 at 37° C. (reaction volume 2.625 ml)”. It is not expected that this unit definition will change in the future, and so commercially obtainable Benzonase® that has been QC checked against this standard unit activity can be used in the DNAse Alert assay to verify secreted nuclease activity achieved in the invention. Should for any reason suppliers of Benzonase® alter the unit definition then it is expected that the relationship between ‘old’ and ‘new’ unit definitions will be known, and therefore also applied to secreted nuclease activity reported and claimed within this invention.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments.

1-82. (canceled)

83. A eukaryotic viral vector production cell comprising nucleic acid sequences expressing: 1) viral vector components; and 2) a nuclease, wherein the nuclease is capable of degrading residual nucleic acid during viral vector production.

84. The eukaryotic viral vector production cell of claim 83, wherein the nuclease comprises a secretory signal.

85. The eukaryotic viral vector production cell according to claim 83, wherein the nuclease comprises a cell retention signal.

86. The eukaryotic viral vector production cell of claim 83, wherein the nuclease is an extracellular nuclease, a sugar-non-specific nuclease, or a salt-active nuclease.

87. The eukaryotic viral vector production cell of claim 83, wherein the nuclease comprises an endonuclease, an exonuclease, or an endonuclease domain fused to an exonuclease domain.

88. The eukaryotic viral vector production cell of claim 87, wherein the endonuclease domain is a VcEndA.

89. The eukaryotic viral vector production cell of claim 83, wherein the nuclease comprises: Vibrio cholerae Endonuclease I (SEQ ID NO: 1), Vibrio salmonicida Endonuclease I (SEQ ID NO: 2), Serratia marcescens Nuclease A (SEQ ID NO: 3), BacNucB (SEQ ID NO: 4), VcEndA-12glc (SEQ ID NO: 5), VcEndA-123glc (SEQ ID NO: 6), VcEndA-124glc (SEQ ID NO: 7), VcEndA-134glc (SEQ ID NO: 8), VcEndA-13glc (SEQ ID NO: 9), VcEndA-14glc (SEQ ID NO: 10), or VcEndA-1glc (SEQ ID NO: 11).

90. The viral vector production cell of claim 83, wherein the nucleic acid encoding the nuclease comprises an inducible or conditional promoter or regulatory element.

91. The eukaryotic viral vector production cell of claim 83, wherein the viral vector components comprise a nucleotide sequence of interest (NOI).

92. The eukaryotic viral vector production cell of claim 83, wherein the viral vector components are retroviral vector components.

93. The viral vector production cell of claim 83, wherein the viral vector components comprise:
i) gag-pol;
ii) env;
iii) optionally the RNA genome of a retroviral vector; and
iv) optionally rev, or a functional substitute thereof.

94. A viral vector production system comprising a co-culture of: 1) a eukaryotic viral vector production cell comprising nucleic acid sequences expressing viral vector components; and 2) a nuclease helper cell expressing a nucleic acid sequence encoding a nuclease, wherein the nuclease comprises a secretory signal, and wherein the nuclease is capable of degrading residual nucleic acid during viral vector production.

95. The viral vector production system of claim 94, wherein the nuclease is an extracellular nuclease, a sugar-non-specific nuclease, or a salt-active nuclease.

96. The viral vector production system of claim 94, wherein the nuclease is an endonuclease, an exonuclease, or an endonuclease domain fused to an exonuclease domain.

97. The viral vector production system of claim 96, wherein the endonuclease domain is a VcEndA.

98. The viral vector production system of claim 94, wherein the nuclease comprises: Vibrio cholerae Endonuclease I (SEQ ID NO: 1), Vibrio salmonicida Endonuclease I (SEQ ID NO: 2), Serratia marcescens Nuclease A (SEQ ID NO: 3), BacNucB (SEQ ID NO: 4), VcEndA-12glc (SEQ ID NO: 5), VcEndA-123glc (SEQ ID NO: 6), VcEndA-124glc (SEQ ID NO: 7), VcEndA-134glc (SEQ ID NO: 8), VcEndA-13glc (SEQ ID NO: 9), VcEndA-14glc (SEQ ID NO: 10), or VcEndA-1glc (SEQ ID NO: 11).

99. The viral vector production system of claim 94, wherein the nucleic acid encoding the nuclease comprises an inducible or conditional promoter or regulatory element.

100. The viral vector production system of claim 94, wherein the viral vector components comprise a nucleotide sequence of interest (NOI).

101. The viral vector production system of claim 94, wherein the viral vector components are retroviral vector components.

102. The viral vector production system of claim 94, wherein the viral vector components comprise:
i) gag-pol;
ii) env;
iii) optionally the RNA genome of a retroviral vector; and
iv) optionally rev, or a functional substitute thereof.

103. A secreted nuclease comprising the amino acid sequence of SEQ ID NO: 11, wherein the secreted nuclease is capable of degrading residual nucleic acid during viral vector production.

104. A method of producing the viral vector production cell of claim 83, the method comprising transfecting a eukaryotic cell with nucleic acid sequences encoding: 1) viral vector components, wherein the viral vector components are expressed in the cell; and 2) a nuclease, wherein the nuclease is capable of degrading residual nucleic acid during viral vector production.

105. A method of producing a viral vector, the method comprising culturing the eukaryotic viral vector production cell of claim 83 in cell culture media under conditions suitable for production of a viral vector, wherein the nuclease is secreted by the eukaryotic viral vector production cell, and wherein residual nucleic acid in the cell culture media is degraded by the nuclease.

106. A method of producing a viral vector, the method comprising culturing the viral vector production system of claim 94 in cell culture media under conditions suitable for production of a viral vector, wherein the nuclease is secreted by the nuclease helper cell, and wherein residual nucleic acid in the cell culture media is degraded by the nuclease.

107. A method of producing a viral vector, the method comprising: (1) culturing in cell culture media, under conditions suitable for production of a viral vector, a eukaryotic viral vector production cell expressing viral vector components; and (2) contacting the eukaryotic viral vector production cell with a liquid feed from a eukaryotic nuclease helper cell, wherein the liquid feed comprises nuclease expressed and secreted by the eukaryotic nuclease helper cell, and wherein residual nucleic acid in the cell culture media is degraded by the nuclease.


SEQUENCE LISTING (29 sequences)

SEQ ID NO: 1 - protein, 231 aa, Vibrio cholerae
MMIFRFVTTLAASLPLLTFAAPISFSHAKNEAVKIYRDHPVSFYCGCEIRWQGKKGIPDLESCGYQVRKNENRASRIEWEHVVPAWQFGHQLQCWQQGGRKNCTRTSPEFNQMEADLHNLTPAIGEVNGNRSNFSFSQWNGIDGVTYGQCEMQVNFKERTAMPPERARGAIARTYLYMSEQYGLRLSKAQNQLMQAWNNQYPVSEWECVRDQKIEKVQGNSNRFVREQCPN

SEQ ID NO: 2 - protein, 234 aa, Vibrio sp.
  source 1..234: note = Vibrio salmonicida
MKLIRLVISLIAVSFTVNVMAAPPSSFSKAKKEAVKIYLDYPTSFYCGCDITWKNKKKGIPELESCGYQVRKQEKRASRIEWEHVVPAWQFGHQRQCWQKGGRKNCTRNDKQFKSMEADLHNLVPAIGEVNGDRSNFRFSQWNGSKGAFYGQCAFKVDFKGRVAEPPAQSRGAIARTYLYMNNEYKFNLSKAQRQLMEAWNKQYPVSTWECTRDERIAKIQGNHNQFVYKACTK

SEQ ID NO: 3 - protein, 266 aa, Serratia marcescens
MRFNNKMLALAALLFAAQASADTLESIDNCAVGCPTGGSSNVSIVRHAYTLNNNSTTKFANWVAYHITKDTPASGKTRNWKTDPALNPADTLAPADYTGANAALKVDRGHQAPLASLAGVSDWESLNYLSNITPQKSDLNQGAWARLEDQERKLIDRADISSVYTVTGPLYERDMGKLPGTQKAHTIPSAYWKVIFINNSPAVNHYAAFLFDQNTPKGADFCQFRVTVDEIEKRTGLIIWAGLPDDVQASLKSKPGVLPELMGCKN

SEQ ID NO: 4 - protein, 142 aa, Bacillus licheniformis
MIKKWAVHLLFSALVLLGLSGGAAYSPQHAEGAARYDDILYFPASRYPETGAHISDAIKAGHSDVCTIERSGADKRRQESLKGIPTKPGFDRDEWPMAMCEEGGKGASVRYVSSSDNRGAGSWVGNRLSGFADGTRILFIVQ

SEQ ID NO: 5 - protein, 231 aa, synthetic construct
MMIFRFVTTLAASLPLLTFAAPISFSHAKNEAVKIYRDHPVSFYCGCEIRWQGKKGIPDLESCGYQVRKNENRASRIEWEHVVPAWQFGHQLQCWQQGGRKNCTRTSPEFNQMEADLHNLTPAIGEVNGDRSNFRFSQWNGIDGVTYGQCEMQVNFKERTAMPPERARGAIARTYLYMSEQYGLRLSKAQNQLMQAWNNQYPVSEWECVRDQKIEKVQGNSNRFVREQCPN

SEQ ID NO: 6 - protein, 231 aa, synthetic construct
MMIFRFVTTLAASLPLLTFAAPISFSHAKNEAVKIYRDHPVSFYCGCEIRWQGKKGIPDLESCGYQVRKNENRASRIEWEHVVPAWQFGHQLQCWQQGGRKNCTRTSPEFNQMEADLHNLTPAIGEVNGNRSNFRFSQWNGIDGVTYGQCEMQVNFKERTAMPPERARGAIARTYLYMSEQYGLRLSKAQNQLMQAWNNQYPVSEWECVRDQKIEKVQGNSNRFVREQCPN

SEQ ID NO: 7 - protein, 231 aa, synthetic construct
MMIFRFVTTLAASLPLLTFAAPISFSHAKNEAVKIYRDHPVSFYCGCEIRWQGKKGIPDLESCGYQVRKNENRASRIEWEHVVPAWQFGHQLQCWQQGGRKNCTRTSPEFNQMEADLHNLTPAIGEVNGDRSNFSFSQWNGIDGVTYGQCEMQVNFKERTAMPPERARGAIARTYLYMSEQYGLRLSKAQNQLMQAWNNQYPVSEWECVRDQKIEKVQGNSNRFVREQCPN

SEQ ID NO: 8 - protein, 231 aa, synthetic construct
MMIFRFVTTLAASLPLLTFAAPISFSHAKNEAVKIYRDHPVSFYCGCEIRWQGKKGIPDLESCGYQVRKNENRASRIEWEHVVPAWQFGHQLQCWQQGGRKNCTRTSPEFNQMEADLHNLVPAIGEVNGNRSNFSFSQWNGIDGVTYGQCEMQVNFKERTAMPPERARGAIARTYLYMSEQYGLRLSKAQNQLMQAWNNQYPVSEWECVRDQKIEKVQGNSNRFVREQCPN

SEQ ID NO: 9 - protein, 231 aa, synthetic construct
MMIFRFVTTLAASLPLLTFAAPISFSHAKNEAVKIYRDHPVSFYCGCEIRWQGKKGIPDLESCGYQVRKNENRASRIEWEHVVPAWQFGHQLQCWQQGGRKNCTRTSPEFNQMEADLHNLVPAIGEVNGNRSNFRFSQWNGIDGVTYGQCEMQVNFKERTAMPPERARGAIARTYLYMSEQYGLRLSKAQNQLMQAWNNQYPVSEWECVRDQKIEKVQGNSNRFVREQCPN

SEQ ID NO: 10 - protein, 231 aa, synthetic construct
MMIFRFVTTLAASLPLLTFAAPISFSHAKNEAVKIYRDHPVSFYCGCEIRWQGKKGIPDLESCGYQVRKNENRASRIEWEHVVPAWQFGHQLQCWQQGGRKNCTRTSPEFNQMEADLHNLVPAIGEVNGDRSNFSFSQWNGIDGVTYGQCEMQVNFKERTAMPPERARGAIARTYLYMSEQYGLRLSKAQNQLMQAWNNQYPVSEWECVRDQKIEKVQGNSNRFVREQCPN

SEQ ID NO: 11 - protein, 231 aa, synthetic construct
MMIFRFVTTLAASLPLLTFAAPISFSHAKNEAVKIYRDHPVSFYCGCEIRWQGKKGIPDLESCGYQVRKNENRASRIEWEHVVPAWQFGHQLQCWQQGGRKNCTRTSPEFNQMEADLHNLVPAIGEVNGDRSNFRFSQWNGIDGVTYGQCEMQVNFKERTAMPPERARGAIARTYLYMSEQYGLRLSKAQNQLMQAWNNQYPVSEWECVRDQKIEKVQGNSNRFVREQCPN

SEQ ID NO: 12 - protein, 21 aa, synthetic construct
  REGION 1..21: note = signal peptide
MWWRLWWLLLLLLLLWPMVWA

SEQ ID NO: 13 - protein, 20 aa, Mus musculus
METDTLLLWVLLLWVPGSTG

SEQ ID NO: 14 - protein, 22 aa, Homo sapiens
MDMRVPAQLLGLLLLWLRGARC

SEQ ID NO: 15 - protein, 16 aa, Homo sapiens
MPLLLLLPLLWAGALA

SEQ ID NO: 16 - protein, 23 aa, Homo sapiens
MDAMKRGLCCVLLLCGAVFVSPS

SEQ ID NO: 17 - protein, 16 aa, Homo sapiens
MKWVTFISLLFSSAYS

SEQ ID NO: 18 - protein, 16 aa, unidentified
  source 1..16: note = Vesicular stomatitis virus
MKCLLYLAFLFIGVNC

SEQ ID NO: 19 - protein, 4 aa, synthetic construct
  VARIANT 1: note = K can be replaced by R, H, Q, S or A
  VARIANT 2: note = D can be replaced by E, N or Q
KDEL

SEQ ID NO: 20 - skipped

SEQ ID NO: 21 - protein, 4 aa, synthetic construct
  REGION 1..4: note = retention signal
KDEL

SEQ ID NO: 22 - DNA, 20 bp, synthetic construct
  misc_feature 1..20: note = primer
  source 1..20: mol_type = other DNA
accgcagctaggaataatgg

SEQ ID NO: 23 - DNA, 20 bp, synthetic construct
  misc_feature 1..20: note = primer
  source 1..20: mol_type = other DNA
ctcagttccgaaaaccaaca

SEQ ID NO: 24 - DNA, 20 bp, synthetic construct
  misc_feature 1..20: note = primer
  source 1..20: mol_type = other DNA
agatggattgcacgcaggtt

SEQ ID NO: 25 - DNA, 21 bp, synthetic construct
  misc_feature 1..21: note = primer
  source 1..21: mol_type = other DNA
tgcccagtcatagccgaatag

SEQ ID NO: 26 - DNA, 19 bp, synthetic construct
  misc_feature 1..19: note = probe
  source 1..19: mol_type = other DNA
ctccacccaagcggccgga

SEQ ID NO: 27 - protein, 6 aa, synthetic construct
HHHHHH

SEQ ID NO: 28 - protein, 142 aa, synthetic construct
MIKEEEEEHHHHHHHHHHHHGGAAYSHHHHEGAARYDEEEEEPASRYHHHHHHHHHHHHHGHSDEEEEERSGHHHHHHHHLKGIPTKPGFDRDEWHHHHHHHGGKGAEEEEESSSDNRGAGSWVGNRLSGFADGTEEEEEEQ

SEQ ID NO: 29 - protein, 142 aa, synthetic construct
MIKKWAVHLLFSALVLLGLSGGAAYSPQHAEGAARYDDILYFNASRYPETGAHISDAIKANHSDNCTIERSGADKRRQESLKGIPTKPGFDRDEWPMAMCEEGGKGASVRYVSSSDNRGAGSWVGNRLSGFANGTRILFIVQ